SEQUENCE LISTING (84 sequences)

SEQ ID NO: 1 - protein, 17 aa, homo sapiens
TVFRSSREKRRSADIFI

SEQ ID NO: 2 - protein, 16 aa, Artificial Sequence
  synthetic
TVFRSSEXRRSADIFI

SEQ ID NO: 3 - protein, 16 aa, Artificial Sequence
  synthetic
TFFRSSEXRRSADIFI

SEQ ID NO: 4 - protein, 16 aa, Artificial Sequence
  synthetic
TVFRSSEXFRSADIFI

SEQ ID NO: 5 - protein, 16 aa, Artificial Sequence
  synthetic
TVFRSSEXRRSAFIFI

SEQ ID NO: 6 - protein, 16 aa, Artificial Sequence
  synthetic
TVFRSSEXRRSADFFI

SEQ ID NO: 7 - protein, 16 aa, Artificial Sequence
  synthetic
TVFRSSEXRRSADIFF

SEQ ID NO: 8 - protein, 16 aa, Artificial Sequence
  synthetic
HVFRSSEXRRSADIFI

SEQ ID NO: 9 - protein, 16 aa, Artificial Sequence
  synthetic
THFRSSEXRRSADIFI

SEQ ID NO: 10 - protein, 16 aa, Artificial Sequence
  synthetic
TVHRSSEXRRSADIFI

SEQ ID NO: 11 - protein, 16 aa, Artificial Sequence
  synthetic
TVFHSSEXRRSADIFI

SEQ ID NO: 12 - protein, 16 aa, Artificial Sequence
  synthetic
TVFRSHEXRRSADIFI

SEQ ID NO: 13 - protein, 16 aa, Artificial Sequence
  synthetic
TVFRSSHXRRSADIFI

SEQ ID NO: 14 - protein, 16 aa, Artificial Sequence
  synthetic
TVFRSSEXHRSADIFI

SEQ ID NO: 15 - protein, 16 aa, Artificial Sequence
  synthetic
TVFRSSEXRHSADIFI

SEQ ID NO: 16 - protein, 16 aa, Artificial Sequence
  synthetic
TVFRSSEXRRSHDIFI

SEQ ID NO: 17 - protein, 16 aa, Artificial Sequence
  synthetic
TVFRSSEXRRSAHIFI

SEQ ID NO: 18 - protein, 16 aa, Artificial Sequence
  synthetic
TVFRSSEXRRSADHFI

SEQ ID NO: 19 - protein, 16 aa, Artificial Sequence
  synthetic
TVFRSSEXRRSADIHI

SEQ ID NO: 20 - protein, 16 aa, Artificial Sequence
  synthetic
TVFRSSEXRRSADIFH

SEQ ID NO: 21 - protein, 16 aa, Artificial Sequence
  synthetic
QVFRSSEXRRSADIFI

SEQ ID NO: 22 - protein, 16 aa, Artificial Sequence
  synthetic
TQFRSSEXRRSADIFI

SEQ ID NO: 23 - protein, 16 aa, Artificial Sequence
  synthetic
TVFQSSEXRRSADIFI

SEQ ID NO: 24 - protein, 16 aa, Artificial Sequence
  synthetic
TVFRQSEXRRSADIFI

SEQ ID NO: 25 - protein, 16 aa, Artificial Sequence
  synthetic
TVFRSQEXRRSADIFI

SEQ ID NO: 26 - protein, 16 aa, Artificial Sequence
  synthetic
TVFRSSQXRRSADIFI

SEQ ID NO: 27 - protein, 16 aa, Artificial Sequence
  synthetic
TVFRSSEXQRSADIFI

SEQ ID NO: 28 - protein, 16 aa, Artificial Sequence
  synthetic
TVFRSSEXRQSADIFI

SEQ ID NO: 29 - protein, 16 aa, Artificial Sequence
  synthetic
TVFRSSEXRRSQDIFI

SEQ ID NO: 30 - protein, 16 aa, Artificial Sequence
  synthetic
TVFRSSEXRRSAQIFI

SEQ ID NO: 31 - protein, 16 aa, Artificial Sequence
  synthetic
TVFRSSEXRRSADQFI

SEQ ID NO: 32 - protein, 16 aa, Artificial Sequence
  synthetic
TVFRSSEXRRSADIQI

SEQ ID NO: 33 - protein, 16 aa, Artificial Sequence
  synthetic
TVFRSSEXRRSADIFQ

SEQ ID NO: 34 - protein, 16 aa, Artificial Sequence
  synthetic
DVFRSSEXRRSADIFI

SEQ ID NO: 35 - protein, 16 aa, Artificial Sequence
  synthetic
TDFRSSEXRRSADIFI

SEQ ID NO: 36 - protein, 16 aa, Artificial Sequence
  synthetic
TVDRSSEXRRSADIFI

SEQ ID NO: 37 - protein, 16 aa, Artificial Sequence
  synthetic
TVFRDSEXRRSADIFI

SEQ ID NO: 38 - protein, 16 aa, Artificial Sequence
  synthetic
TVFRSDEXRRSADIFI

SEQ ID NO: 39 - protein, 16 aa, Artificial Sequence
  synthetic
TVFRSSEXRRDADIFI

SEQ ID NO: 40 - protein, 16 aa, Artificial Sequence
  synthetic
TVFRSSEXRRSDDIFI

SEQ ID NO: 41 - protein, 16 aa, Artificial Sequence
  synthetic
TVFRSSEXRRSADDFI

SEQ ID NO: 42 - protein, 16 aa, Artificial Sequence
  synthetic
TVFRSSEXRRSADIFD

SEQ ID NO: 43 - protein, 16 aa, Artificial Sequence
  synthetic
PVFRSSEXRRSADIFI

SEQ ID NO: 44 - protein, 16 aa, Artificial Sequence
  synthetic
TPFRSSEXRRSADIFI

SEQ ID NO: 45 - protein, 16 aa, Artificial Sequence
  synthetic
TVFPSSEXRRSADIFI

SEQ ID NO: 46 - protein, 16 aa, Artificial Sequence
  synthetic
TVFRPSEXRRSADIFI

SEQ ID NO: 47 - protein, 16 aa, Artificial Sequence
  synthetic
TVFRSPEXRRSADIFI

SEQ ID NO: 48 - protein, 16 aa, Artificial Sequence
  synthetic
TVFRSSPXRRSADIFI

SEQ ID NO: 49 - protein, 16 aa, Artificial Sequence
  synthetic
TVFRSSEPRRSADIFI

SEQ ID NO: 50 - protein, 16 aa, Artificial Sequence
  synthetic
TVFRSSEXPRSADIFI

SEQ ID NO: 51 - protein, 16 aa, Artificial Sequence
  synthetic
TVFRSSEXRPSADIFI

SEQ ID NO: 52 - protein, 16 aa, Artificial Sequence
  synthetic
TVFRSSEXRRPADIFI

SEQ ID NO: 53 - protein, 16 aa, Artificial Sequence
  synthetic
TVFRSSEXRRSPDIFI

SEQ ID NO: 54 - protein, 16 aa, Artificial Sequence
  synthetic
TVFRSSEXRRSAPIFI

SEQ ID NO: 55 - protein, 16 aa, Artificial Sequence
  synthetic
TVFRSSEXRRSADPFI

SEQ ID NO: 56 - protein, 16 aa, Artificial Sequence
  synthetic
TVFRSSEXRRSADIPI

SEQ ID NO: 57 - protein, 16 aa, Artificial Sequence
  synthetic
TVFRSSEXRRSADIFP

SEQ ID NO: 58 - protein, 16 aa, Artificial Sequence
  synthetic
TXFRSSEXRRSADIFI

SEQ ID NO: 59 - protein, 16 aa, Artificial Sequence
  synthetic
TVFXSSEXRRSADIFI

SEQ ID NO: 60 - protein, 16 aa, Artificial Sequence
  synthetic
TVFRSXEXRRSADIFI

SEQ ID NO: 61 - protein, 16 aa, Artificial Sequence
  synthetic
TVFRSSEXRRSADIFI

SEQ ID NO: 62 - protein, 16 aa, Artificial Sequence
  synthetic
TVFRSSEXXRSADIFI

SEQ ID NO: 63 - protein, 16 aa, Artificial Sequence
  synthetic
TVFRSSEXRXSADIFI

SEQ ID NO: 64 - protein, 16 aa, Artificial Sequence
  synthetic
TVFRSSEXRRXADIFI

SEQ ID NO: 65 - protein, 16 aa, Artificial Sequence
  synthetic
TVFRSSEXRRSXDIFI

SEQ ID NO: 66 - protein, 16 aa, Artificial Sequence
  synthetic
TVFRSSEXRRSAXIFI

SEQ ID NO: 67 - protein, 16 aa, Artificial Sequence
  synthetic
TVFRSSEXRRSADIXI

SEQ ID NO: 68 - protein, 16 aa, Artificial Sequence
  synthetic
TVFRSSEXRRSADIFX

SEQ ID NO: 69 - protein, 16 aa, Artificial Sequence
  synthetic
GVFRSSEXRRSADIFI

SEQ ID NO: 70 - protein, 16 aa, Artificial Sequence
  synthetic
TGFRSSEXRRSADIFI

SEQ ID NO: 71 - protein, 16 aa, Artificial Sequence
  synthetic
TVGRSSEXRRSADIFI

SEQ ID NO: 72 - protein, 16 aa, Artificial Sequence
  synthetic
TVFGSSEXRRSADIFI

SEQ ID NO: 73 - protein, 16 aa, Artificial Sequence
  synthetic
TVFRGSEXRRSADIFI

SEQ ID NO: 74 - protein, 16 aa, Artificial Sequence
  synthetic
TVFRSGEXRRSADIFI

SEQ ID NO: 75 - protein, 16 aa, Artificial Sequence
  synthetic
TVFRSSGXRRSADIFI

SEQ ID NO: 76 - protein, 16 aa, Artificial Sequence
  synthetic
TVFRSSEXGRSADIFI

SEQ ID NO: 77 - protein, 16 aa, Artificial Sequence
  synthetic
TVFRSSEXRGSADIFI

SEQ ID NO: 78 - protein, 16 aa, Artificial Sequence
  synthetic
TVFRSSEXRRGADIFI

SEQ ID NO: 79 - protein, 16 aa, Artificial Sequence
  synthetic
TVFRSSEXRRSAGIFI

SEQ ID NO: 80 - protein, 16 aa, Artificial Sequence
  synthetic
TVFRSSEXRRSADIGI

SEQ ID NO: 81 - protein, 16 aa, Artificial Sequence
  synthetic
TVFRSSEXRRSADIFG

SEQ ID NO: 82 - protein, 16 aa, Artificial Sequence
  synthetic
TVDRSSEXRRSADIFI

SEQ ID NO: 83 - protein, 16 aa, Artificial Sequence
  synthetic
TVDRSSEKRRSADIFI

SEQ ID NO: 84 - protein, 15 aa, Artificial Sequence
  synthetic
TVFQSEXRRSADQFI

(19) United States
(12) Patent Application Publication
(10) Pub. No.: US 2015/0011466 A1
(43) Pub. Date: Jan. 8, 2015

(54) APJ Receptor Compounds

(71) Applicant: Anchor Therapeutics, Inc., Cambridge, MA (US)

(72) Inventors: Thomas J. McMurry, Winchester, MA (US); Richard J. Looby, Reading, MA (US)

(21) Appl. No.: 14/371,361

(22) PCT Filed: Jan. 9, 2013
(86) PCT No.: PCT/US13/20842

§ 371 Date: Jul. 9, 2014

Related U.S. Application Data
(60) Provisional application No. 61/584,785, filed on Jan. 9, 2012.

(51) Int. Cl. C07K 7/08

(52) U.S. Cl. CPC C07K 7/08
USPC 514/3.8; 530/326; 514/21.4; 514/19.3; 514/6.9; 514/4.8

(57) ABSTRACT

The invention relates generally to compounds which are allosteric modulators (e.g., negative and positive allosteric modulators, allosteric agonists, and ago-allosteric modulators) of the G protein coupled receptor apelin, also known as the APJ receptor. The APJ receptor compounds are derived from the it intracellular loop and domain of the APJ receptor. The invention also relates to the use of these APJ receptor compounds and pharmaceutical compositions comprising the APJ receptor compounds in the treatment of diseases and conditions associated with APJ receptor modulation, such as cardiovascular diseases, (e.g., hypertension and heart failure, such as congestive heart failure), cancer, diabetes, stem cell trafficking, fluid homeostasis, cell proliferation, immune function, obesity, metastatic disease, and HIV infection.

### RELATED APPLICATION

This application claims the benefit of U.S. Provisional Application No. 61/584,785, filed on Jan. 9, 2012.

The entire teachings of the above application are incorporated herein by reference.

### BACKGROUND OF THE INVENTION

G protein coupled receptors (GPCRs) constitute one of the largest families of genes in the human genome. GPCRs are integral membrane signaling proteins. Hydrophobicity mapping of the amino acid sequences of G-protein coupled receptors has led to a model of the typical G-protein-coupled receptor as containing seven hydrophobic membrane-spanning regions with the amino terminal on the extracellular side of the membrane and the carboxyl terminal on the intracellular side of the membrane.

GPCRs mediate the transmission of intracellular signals (“signal transduction”) by activating guanine nucleotide-binding proteins (G proteins) to which the receptor is coupled. GPCRs are activated by a wide range of endogenous stimuli, including peptides, amino acids. hormones, light, and metal ions. The following reviews are incorporated by reference: Hill, British J. Pharm 147: s27 (2006); Palczeski, Ann Rev Biochemistry 75: 743-767 (2006); Dorsham & Gutkind, Nature Reviews 7: 79-94 (2007); Kobilka & Schertler, Trends Pharmacol Sci. 2: 79-83 (2008).

GPCRs are important targets for drug discovery as they are involved in a wide range of cellular signaling pathways and are implicated in many pathological conditions (e.g., cardiovascular and mental disorders, cancer, AIDS). In fact, GPCRs are targeted by 40-50% of approved drugs, illustrating the critical importance of this class of pharmaceutical targets. Interestingly, this number represents only about 30 GPCRs, a small fraction of the total number of GPCRs thought to be relevant to human disease. Over 1000 GPCRs are known in the human genome, and GPCRs remain challenging targets from a research and development perspective in part because these amembrane bound receptors with complex pharmacology.

There remains a need for the development of new pharmaceuticals that are allosteric modulators of GPCRs (e.g., negative and positive allosteric modulators, allosteric agonists, and ago-allosteric modulators).

### SUMMARY OF THE INVENTION

The invention relates generally to compounds which are allosteric modulators (e.g., negative and positive allosteric modulators, allosteric agonists, and ago-allosteric modulators) of the G protein coupled receptor apelin, also known as the APJ receptor. The APJ receptor compounds are derived from the intracellular loops and domains of the the APJ receptor. The invention also relates to the use of these APJ receptor compounds and pharmaceutical compositions comprising the APJ receptor compounds in the treatment of diseases and conditions associated with APJ receptor modulation, such as cardiovascular diseases, (e.g., hypertension and heart failure, such as congestive heart failure), cancer, diabetes, stem cell trafficking, fluid homeostasis, cell proliferation, immune function, obesity, metastatic disease, and HIV infection. In another embodiment, the compounds of the invention are combined with a second therapeutic agent. The second therapeutic is an agent useful in the treatment or prevention of a disease or condition selected from hypertension and heart failure, in particular, congestive heart failure and hypertrophic cardiomyopathy. In addition, the second therapeutic would be useful in the treatment or prevention of coronary artery disease, atherosclerosis, stable and unstable angina pectoris, restenosis, acute myocardial infarction, pulmonary hypertension, diseases related to cardiac ischemia, and sudden heart death.

More specifically, the compounds of the invention are represented by Formula A:

T-L-X₁-X₂-X₃-X₄-X₅-X₆-X₇-X₈-X₉-X₁₀-X₁₁-X₁₂-X₁₃-X₁₄-X₁₅-X₁₆-R₁;

or a pharmaceutically acceptable salt thereof, wherein L is a linking moiety bonded to the N terminal nitrogen of X₁ or the next present amino acid residue if X₁ is absent and is selected from: C*(O), C*(S), S*(O)₂, N(R¹³)S*(O), N(R¹³)S*(O)₂, N(R¹³)C*(O), N(R¹³)C*(S), OC*(O), OC*(S), SC*(O), SC*(S), C*(═NH), and N(R¹³)C*(═NH); wherein L is bonded to X₁ or the next present amino acid residue if X₁ is absent at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; T is a lipophilic moiety bonded to L; R₁ is —OR₂—, or NR₃R₄; wherein

each R₂ is hydrogen or a (C₁-C₁₀) alkyl group,

R₃ and R₄ are each independently selected from hydrogen, (C₁-C₁₀) alkyl, (C₁-C₁₀)aralkyl, [CH₂CH₂O]ₙCH₂CH₂C(O)OR₂ or [CH₂CH₂O]ₙCH₂CH₂C(O)NR₂;

n is 1-20

or —NR₃R₄ is a non-aromatic nitrogen-containing heterocyclic group, wherein the non-aromatic group is optionally mono-or di-substituted at one or more substitutable carbon atoms with an R₅;

each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂;
X₁ is absent, a threonine residue, a histidine residue, a glutamine residue, an aspartic acid residue, an Aib residue, a phenylalanine residue, or a glycine residue;
X₂ is absent, a valine residue, a phenylalanine residue, a histadine residue, an Aib residue, a glutamine residue, an aspartic acid residue, a proline residue, or a glycine residue;
X₃ is absent, a phenylalanine residue, a histidine residue, an aspartic acid residue, a glycine residue, or a proline residue;
X₄ is absent, an arginine residue, a phenylalanine residue, a histidine residue, a glutamine residue, an aspartic acid residue, an Aib residue, a proline residue, or a glycine residue;
X₅ is absent, a serine residue, a phenylalanine residue, a histidine residue, a glutamine residue, an aspartic acid residue, a proline residue, an Aib residue, or a glycine residue;
X₆ is absent, a serine residue, a phenylalanine residue, a histidine residue, a glutamine residue, an aspartic acid residue, a proline residue, an Aib residue, or a glycine residue;
X₇ is absent, a glutamic acid, a phenylalanine residue, a histidine residue, a glutamine residue, an aspartic acid residue, a proline residue, an Aib residue, or a glycine residue;
X₈ is absent, a lysine, D-lysine residue, a phenylalanine residue, a proline residue, a glutamic acid, a phenylalanine residue, a histidine residue, a glutamine residue, an aspartic acid residue, an Aib residue, or a glycine residue, or a tyrosine residue;
X₉ is absent, an arginine residue, a phenylalanine residue, a histidine residue, a glutamine residue, an aspartic acid residue, a proline residue, an Aib residue, or a glycine residue;
X₁₀ is absent, an arginine residue, a phenylalanine residue, a histidine residue, a glutamine residue, an aspartic acid residue, a proline residue, an Aib residue, or a glycine residue;
X₁₁ is absent, a serine residue, a phenylalanine residue, a histidine residue, a glutamine residue, an aspartic acid residue, a proline residue, an Aib residue, or a glycine residue;
X₁₂ is absent or an alanine residue, a phenylalanine residue, a histidine residue, a glutamine residue, an aspartic acid residue, a proline residue, an Aib residue, or a glycine residue;
X₁₃ is absent, an aspartic acid residue, a phenylalanine residue, a histidine residue, a glutamine residue, an aspartic acid residue, a proline residue, an Aib residue, or a glycine residue;
X₁₄ is absent, an isoleucine residue, a phenylalanine residue, a histidine residue, a glutamine residue, an aspartic acid residue, a proline residue, an Aib residue, or a glycine residue;
X₁₅ is absent, a phenylalanine residue, a histidine residue, a glutamine residue, an aspartic acid residue, a proline residue, an Aib residue, or a glycine residue;
X₁₆ is absent, a phenylalanine residue, an isoleucine residue, a phenylalanine residue, a histidine residue, a glutamine residue, an aspartic acid residue, a proline residue, an Aib residue, or a glycine residue;
wherein at least three of X₁-X₁₆ are present and contiguous and optionally 1-5 amino acid residues are present in the D configuration.
   In another aspect of the invention the compounds are represented by Formula B:

T-L-X₁′-X₂-X₃-X₄-X₅-X₆-X₇-X₈-X₉-X₁₀-X₁₁-X₁₂-X₁₃-X₁₄-X₁₅-X₁₆-R₁;

or a pharmaceutically acceptable salt thereof, wherein L is a linking moiety bonded to the N terminal nitrogen of X₁′ or the next present amino acid residue if X₁′ is absent and is selected from: C*(O), C*(S), S*(O)₂, N(R¹³)S*(O), N(R¹³)S*(O)₂, N(R¹³)C*(O), N(R¹³)C*(S), OC*(O), OC*(S), SC*(O), SC*(S), C*(═NH), and N(R¹³)C*(═NH); wherein L is bonded to X₁′ or the next present amino acid residue if X₁′ is absent at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; T is a lipophilic moiety bonded to L; R₁ is —OR₂—, or NR₃R₄; wherein

each R₂ is hydrogen or a (C₁-C₁₀) alkyl group,

R₃ and R₄ are each independently selected from hydrogen, (C₁-C₁₀) alkyl, (C₁-C₁₀)aralkyl, [CH₂CH₂O]ₙCH₂CH₂C(O)OR₂ or [CH₂CH₂O]ₙCH₂CH₂C(O)NR₂;

n is 1-20

or —NR₃R₄ is a non-aromatic nitrogen-containing heterocyclic group, wherein the non-aromatic group is optionally mono-or di-substituted at one or more substitutable carbon atoms with an R₅;

each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂;
X₁′ is absent, a threonine residue, a histidine residue, a glutamine residue, an aspartic acid residue, an Aib residue, a phenylalanine residue, a glycine residue, or a proline residue;
X₂ is absent, a valine residue, a phenylalanine residue, a histadine residue, an Aib residue, a glutamine residue, an aspartic acid residue, a proline residue, or a glycine residue;
X₃ is absent, a phenylalanine residue, a histidine residue, an aspartic acid residue, a glycine residue, or a proline residue;
X₄ is absent, an arginine residue, a phenylalanine residue, a histidine residue, a glutamine residue, an aspartic acid residue, an Aib residue, a proline residue, or a glycine residue;
X₅ is absent, a serine residue, a phenylalanine residue, a histidine residue, a glutamine residue, an aspartic acid residue, a proline residue, an Aib residue, or a glycine residue;
X₆ is absent, a serine residue, a phenylalanine residue, a histidine residue, a glutamine residue, an aspartic acid residue, a proline residue, an Aib residue, or a glycine residue;
X₇ is absent, a glutamic acid, a phenylalanine residue, a histidine residue, a glutamine residue, an aspartic acid residue, a proline residue, an Aib residue, or a glycine residue;
X₈ is absent, a lysine, D-lysine residue, a phenylalanine residue, a proline residue, a glutamic acid, a phenylalanine residue, a histidine residue, a glutamine residue, an aspartic acid residue, an Aib residue, or a glycine residue, or a tyrosine residue;
X₉ is absent, an arginine residue, a phenylalanine residue, a histidine residue, a glutamine residue, an aspartic acid residue, a proline residue, an Aib residue, or a glycine residue;
X₁₀ is absent, an arginine residue, a phenylalanine residue, a histidine residue, a glutamine residue, an aspartic acid residue, a proline residue, an Aib residue, or a glycine residue;
X₁₁ is absent, a serine residue, a phenylalanine residue, a histidine residue, a glutamine residue, an aspartic acid residue, a proline residue, an Aib residue, or a glycine residue;
X₁₂ is absent or an alanine residue, a phenylalanine residue, a histidine residue, a glutamine residue, an aspartic acid residue, a proline residue, an Aib residue, or a glycine residue;
X₁₃ is absent, an aspartic acid residue, a phenylalanine residue, a histidine residue, a glutamine residue, an aspartic acid residue, a proline residue, an Aib residue, or a glycine residue;
X₁₄ is absent, an isoleucine residue, a phenylalanine residue, a histidine residue, a glutamine residue, an aspartic acid residue, a proline residue, an Aib residue, or a glycine residue;
X₁₅ is absent, a phenylalanine residue, a histidine residue, a glutamine residue, an aspartic acid residue, a proline residue, an Aib residue, or a glycine residue;
X₁₆ is absent, a phenylalanine residue, an isoleucine residue, a phenylalanine residue, a histidine residue, a glutamine residue, an aspartic acid residue, a proline residue, an Aib residue, or a glycine residue;
wherein at least three of X₁′-X₁₆ are present and contiguous and optionally 1-5 amino acid residues are present in the D configuration.
   In another aspect of the invention the compounds are represented by Formula I:

T-L-P,

or pharmaceutically acceptable salts thereof, wherein:
P is a peptide comprising at least three contiguous amino-acid residues of the intracellular i1 loop of the APJ receptor, wherein
L is a linking moiety bonded to P at an N-terminal nitrogen of an N-terminal amino-acid residue selected from: C*(O), C*(S), S*(O)₂, N(R¹³)S*(O), N(R¹³)S*(O)₂, N(R¹³)C*(O), N(R¹³)C*(S), OC*(O), OC*(S), SC*(O), SC*(S), C*(═NH), and N(R¹³)C*(═NH); wherein L is bonded to P at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; and T is a lipophilic tether moiety bonded to L, wherein the C-terminal amino acid residue of P is functionalized by replacement of the acid moiety with C(O)NR₃¹R₄¹; R₃¹ is selected from hydrogen, C₁-C₁₀ alkyl, (C₁-C₁₀)aralkyl, [CH₂CH₂O]ₙCH₂CH₂C(O)OR₂ or [CH₂CH₂O]ₙCH₂CH₂C(O)NR₂; R₄¹ is selected from (C₁-C₁₀)aralkyl, [CH₂CH₂O]ₙCH₂CH₂C(O)OR₂ or [CH₂CH₂O]ₙCH₂CH₂C(O)NR₂;
   n is 1-20

or —NR₃¹R₄¹ is a non-aromatic nitrogen-containing heterocyclic group, wherein the non-aromatic group is optionally mono-or di-substituted at one or more substitutable carbon atoms with an R₅;

each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂.

The invention also relates to pharmaceutical compositions comprising one or more compounds of the invention and a carrier, and the use of the disclosed compounds and compositions in methods of treating diseases and conditions responsive to modulation (inhibition or activation) of the APJ receptor.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A-1E are concentration response curves for selected APJ compounds: Compound 1 (FIG. 1A), Compound 20 (FIG. 1B), Compound 24 (FIG. 1C), Compound 68 (FIG. 1D), and Compound 70 (FIG. 1E). Compounds 1, 20, 24, 68 and 70 inhibited the forskolin analog NKH477 and stimulated increase in cAMP in HEK cells stably expressing the Gi-coupled receptor APJ in a dose dependent manner.

FIGS. 2A-2D are concentration response curves for selected APJ compounds: Endogenous ligand apelin-13 (FIG. 2A), Compound 9 (FIG. 2B), Compound 82 (FIG. 2C), and Compound 87 (FIG. 2E). The endogenous ligand apelin-13 robustly recruits β-arrestin as measured by a dose dependent increase in chemiluminescence. β-arrestin is weakly engaged for Compounds 9, 82, and 87 in a dose dependent manner.

FIGS. 3A-3C compound structures for selected APJ receptor compounds.

### DETAILED DESCRIPTION OF THE INVENTION

A description of example embodiments of the invention follows.

### G Protein Coupled Receptors (GPCRs)

G protein coupled receptors (GPCRs) constitute one of the largest superfamilies of genes in the human genome; these transmembrane proteins enable the cell the respond to its environment by sensing extracellular stimuli and initiating intracellular signal transduction cascades. GPCRs mediate signal transduction through the binding and activation of guanine nucleotide-binding proteins (G proteins) to which the receptor is coupled. Wide arrays of ligands bind to these receptors, which in turn orchestrate signaling networks integral to many cellular functions. Diverse GPCR ligands include small proteins, peptides, amino acids, biogenic amines, lipids, ions, odorants and even photons of light. The following reviews are incorporated by reference: Hill, British J. Pharm 147: s27 (2006); Dorsham & Gutkind, Nature Reviews 7: 79-94 (2007).

In addition to modulating a diverse array of homeostatic processes, GPCR signaling pathways are integral components of many pathological conditions (e.g., cardiovascular and mental disorders, cancer, AIDS). In fact, GPCRs are targeted by 40-50% of approved drugs illustrating the critical importance of this class of pharmaceutical targets. Interestingly, this number represents only about 30 GPCRs, a small fraction of the total number of GPCRs thought to be relevant to human disease. GPCRs are membrane bound receptors that exhibit complex pharmacological properties and remain challenging targets from a research and development perspective. Given their importance in human health combined with their prevalence (over 1000 known GPCRs in the human genome) GPCRs represent an important target receptor class for drug discovery and design.

GPCRs are integral membrane proteins that mediate diverse signaling cascades through an evolutionarily conserved structural motif. All GPCRs are thought to consist of seven hydrophobic transmembrane spanning α-helices with the amino terminus on the extracellular side of the membrane and the carboxyl terminus on the intracellular side of the membrane. The transmembrane helices are linked together sequentially by extracellular (e1, e2, e3) and intracellular (cytoplasmic) loops (i1, i2, i3). The intracellular loops or domains are intimately involved in the coupling and turnover of G proteins and include: i1, which connects TM1-TM2; i2, connecting TM3-TM4; i3, connecting TM5-TM6; and a portion of the C-terminal cytoplasmic tail (domain 4). Due in part to the topological homology of the 7TM domains and the recent high resolution crystal structures of several GPCRs (Palczewski et al., Science 289, 739-45 (2000), Rasmussen, S. G. et al., Nature 450, 383-7 (2007)) skilled modelers are now able to predict the general boundaries of GPCR loop domains through the alignment of several related receptors. These predictions are aided in part by a number of programs used by computational biologists, including EMBOSS, ClustalW2, Kalign, and MAFFT (Multiple Alignment using Fast Fourier Transform). Importantly, many of these programs are publically available (see, for example, The European Bioinformatics Institute (EMBL-EBI) web site http://www.ebi.ac.uk/Tools/) and most have web-based interfaces.

GPCR mediated signal transduction is initiated by the binding of a ligand to its cognate receptor. In many instances GPCR ligand binding is believed to take place in a hydrophilic pocket generated by a cluster of helices near the extracellular domain. However, other ligands, such as large peptides, are thought to bind to the extracellular region of protein and hydrophobic ligands are postulated to intercalate into a receptor binding pocket through the membrane between gaps in the helices. The process of ligand binding induces conformational changes within the receptor. These changes involve the outward movement of helix 6, which in turn alters the conformations of the intracellular loops and ultimately results in a receptor form that is able to bind and activate a heterotrimeric G protein (Farrens, D., et al. Science 274, 768-770 (1996), Gether, U. and Kobilka, B., J. Biol. Chem. 273, 17979-17982 (1998)). Upon binding the receptor catalyzes the exchange of GTP for GDP in the alpha subunit of the heterotrimeric G protein, which results in a separation of the G protein from the receptor as well a dissociation of the alpha and beta/gamma subunits of the G protein itself. Notably, this process is catalytic and results in signal amplification in that activation of one receptor may elicit the activation and turnover of numerous G proteins, which in turn may regulate multiple second messenger systems. Signaling diversity is further achieved through the existence of numerous G protein types as well as differing isoforms of alpha, beta and gamma subunits. Typically, GPCRs interact with G proteins to regulate the synthesis or inhibition of intracellular second messengers such as cyclic AMP, inositol phosphates, diacylglycerol and calcium ions, thereby triggering a cascade of intracellular events that eventually leads to a biological response.

GPCR signaling may be modulated and attenuated through cellular machinery as well as pharmacological intervention. Signal transduction may be ‘switched off’ with relatively fast kinetics (seconds to minutes) by a process called rapid desensitization. For GPCRs, this is caused by a functional uncoupling of receptors from heterotrimeric G proteins, without a detectable change in the total number of receptors present in cells or tissues. This process involves the phosphorylation of the receptor C terminus, which enables the protein Arrestin to bind to the receptor and occulude further G protein coupling. Once bound by Arrestin the receptor may be internalized into the cell and either recycled back to the cell surface or degraded. The alpha subunit of the G protein possesses intrisic GTPase activity, which attenuates signaling and promotes re-association with the beta/gamma subunits and a return to the basal state. GPCR signaling may also be modulated pharmacologically. Agonist drugs act directly to activate the receptors, whereas antagonist drugs act indirectly to block receptor signaling by preventing agonist activity through their associating with the receptor.

GPCR binding and signaling can also be modified through allosteric modulation, that is by ligands that bind not at the orthosteric binding site but through binding at an allosteric site elsewhere in the receptors. Allosteric modulators can include both positive and negative modulators of orthosteric ligand mediated activity, allosteric agonists (that act in the absence of the orthosteric ligand), and ago-allosteric modulators (ligands that have agonist activity on their own but that can also modulate the activity of the orthosteric ligand).

The large superfamily of GPCRs may be divided into subclasses based on structural and functional similarities. GPCR families include Class A Rhodopsin like, Class B Secretin like, Class C Metabotropic glutamate/pheromone, Class D Fungal pheromone, Class E cAMP receptors (Dictyostelium), the Frizzled/Smoothened family, and various orphan GPCRs. In addition, putative families include Ocular albinism proteins, Insect odorant receptors, Plant Mlo receptors, Nematode chemoreceptors, Vomeronasal receptors (VIR & V3R) and taste receptors.

Class A GPCRs, also called family A or rhodopsin-like, are the largest class of receptors and characteristically have relatively small extracellular loops that form the basis for selectivity vs. endogenous agonists and small-molecule drugs. In addition, Class A receptors also have relatively small intracellular loops. Class A receptors include amine family members such as dopamine and serotonin, peptide members such as chemokine and opioid, the visual opsins, odorant receptors and an array of hormone receptors.

The apelin receptor (APJ) is a Class A receptor that has been implicated in conditions such as cardiovascular diseases, such as heart diseases (e.g., hypertension and heart failure, such as congestive heart failure), cancer, diabetes, stem cell trafficking, fluid homeostasis, cell proliferation, immune function, obesity, metastatic disease, and HIV infection.

### Peptides

As defined herein, P is a peptide comprising at least three contiguous amino-acid residues (e.g., at least 3, 4, 5, 6, 7, 8, 9, 10, 11, 12, 13, 14, 15, 16, or 17) of an intracellular i1 loop domain of the apelin (APJ) receptor. In certain embodiments, P is derived from the intercellular loop domain of the apelin (APJ) receptor and is a peptide comprising at least three contiguous amino acid residues (e.g., at least 3, 4, 5, 6, 7, 8, 9, 10, 11, 12, 13, 14, 15, 16, or 17) where the arginine residue at the seventh position in SEQ ID NO. 1 has been deleted. It is understood that, the N-terminal nitrogen of the N-terminal amino acid residue of P to which the linking moiety is bonded can be one of the at least three contiguous amino acid residues or it can be an amino acid residue distinct from the at least three contiguous amino acid residues.

Intracellular i1 loop as used herein refers to the loop which connects TM1 to TM2 and the corresponding transmembrane junctional residues.

In a specific embodiment, P comprises at least three, at least four, at least five, at least six, at least seven, at least eight, at least nine, at least ten, at least eleven, at least twelve, at least thirteen, at least fourteen, at least fifteen, at least sixteen or at least seventeen contiguous amino acid residues of the intracellular i1 loop of the apelin receptor (APJ).

In a more specific embodiment, the at least three contiguous amino acids of P (e.g., at least 3, 4, 5, 6, 7, 8, 9, 10, 11, 12, 13, 14, 15, 16, or 17) are derived are from the intracellular i1 loop of the apelin receptor (APJ), wherein the amino acid sequence of the i1 loop is as described in Table 1.

TABLE 1

| Intracellular | |
|---|---|
| Loop Number | APJ Receptor Intercellular Loop |
| i1 | TVFRSSREKRRSADIFI |
| | (SEQ ID NO: 1) |

It is understood that in addition to the amino acids shown in the sequences in Table 1 or Tables 2a-2c below, the intracellular loop for the i1 loop domain can also include the transmembrane junctional residues. For example, the i1 loop can include SEQ ID NO: 1 where one or more residues from the transmembrane junctional residues are included on either the C-terminus, the N-terminus or both. For example, the amino acid sequence of SEQ ID NO: 1 can further include either an Alanine residue, Serine residue or both in either order at the C-terminus.

In certain embodiments of the invention, P lacks the arginine represented at position 7 of SEQ ID NO: 1, underlined in Table 1, but otherwise comprises at least three, at least four, at least five, at least six, at least seven, at least eight, at least nine, or at least ten contiguous, at least eleven, at least twelve, at least thirteen, at least fourteen, at least fifteen amino acid residues or at least 16 amino acid residues of the i1 intracellular loop of the APJ receptor.

TABLE 2a
APJ Sequence IDs

| SEQ | | | |
|---|---|---|---|
| ID | Loop | Length | Sequence |
| 2 | i1 | 16 | TVFRSSEkRRSADIFI |
| 3 | i1 | 16 | TFFRSSEkRRSADIFI |
| 4 | i1 | 16 | TVFRSSEkFRSADIFI |
| 5 | i1 | 16 | TVFRSSEkRRSAFIFI |
| 6 | i1 | 16 | TVFRSSEkRRSADFFI |
| 7 | i1 | 16 | TVFRSSEkRRSADIFF |
| 8 | i1 | 16 | HVFRSSEkRRSADIFI |
| 9 | i1 | 16 | THFRSSEkRRSADIFI |
| 10 | i1 | 16 | TVHRSSEkRRSADIFI |
| 11 | i1 | 16 | TVFHSSEkRRSADIFI |
| 12 | i1 | 16 | TVFRSHEkRRSADIFI |
| 13 | i1 | 16 | TVFRSSHkRRSADIFI |
| 14 | i1 | 16 | TVFRSSEkHRSADIFI |
| 15 | i1 | 16 | TVFRSSEkRHSADIFI |
| 16 | i1 | 16 | TVFRSSEkRRSHDIFI |
| 17 | i1 | 16 | TVFRSSEkRRSAHIFI |
| 18 | i1 | 16 | TVFRSSEkRRSADHFI |
| 19 | i1 | 16 | TVFRSSEkRRSADIHI |
| 20 | i1 | 16 | TVFRSSEkRRSADIFH |
| 21 | i1 | 16 | QVFRSSEkRRSADIFI |
| 22 | i1 | 16 | TQFRSSEkRRSADIFI |
| 23 | i1 | 16 | TVFQSSEkRRSADIFI |
| 24 | i1 | 16 | TVFRQSEkRRSADIFI |
| 25 | i1 | 16 | TVFRSQEkRRSADIFI |
| 26 | i1 | 16 | TVFRSSQkRRSADIFI |
| 27 | i1 | 16 | TVFRSSEkQRSADIFI |
| 28 | i1 | 16 | TVFRSSEkRQSADIFI |
| 29 | i1 | 16 | TVFRSSEkRRSQDIFI |
| 30 | i1 | 16 | TVFRSSEkRRSAQIFI |
| 31 | i1 | 16 | TVFRSSEkRRSADQFI |
| 32 | i1 | 16 | TVFRSSEkRRSADIQI |
| 33 | i1 | 16 | TVFRSSEkRRSADIFQ |
| 34 | i1 | 16 | DVFRSSEkRRSADIFI |
| 35 | i1 | 16 | TDFRSSEkRRSADIFI |
| 36 | i1 | 16 | TVDRSSEkRRSADIFI |
| 37 | i1 | 16 | TVFRDSEkRRSADIFI |
| 38 | i1 | 16 | TVFRSDEkRRSADIFI |
| 39 | i1 | 16 | TVFRSSEkRRDADIFI |
| 40 | i1 | 16 | TVFRSSEkRRSDDIFI |
| 41 | i1 | 16 | TVFRSSEkRRSADDFI |
| 42 | i1 | 16 | TVFRSSEkRRSADIFD |
| 43 | i1 | 16 | PVFRSSEkRRSADIFI |
| 44 | i1 | 16 | TPFRSSEkRRSADIFI |
| 45 | i1 | 16 | TVFPSSEkRRSADIFI |
| 46 | i1 | 16 | TVFRPSEkRRSADIFI |
| 47 | i1 | 16 | TVFRSPEkRRSADIFI |
| 48 | i1 | 16 | TVFRSSPkRRSADIFI |
| 49 | i1 | 16 | TVFRSSEPRRSADIFI |
| 50 | i1 | 16 | TVFRSSEkPRSADIFI |
| 51 | i1 | 16 | TVFRSSEkRPSADIFI |
| 52 | i1 | 16 | TVFRSSEkRRPADIFI |
| 53 | i1 | 16 | TVFRSSEkRRSPDIFI |
| 54 | i1 | 16 | TVFRSSEkRRSAPIFI |
| 55 | i1 | 16 | TVFRSSEkRRSADPFI |
| 56 | i1 | 16 | TVFRSSEkRRSADIPI |
| 57 | i1 | 16 | TVFRSSEkRRSADIFP |
| 58 | i1 | 16 | T(Aib)FRSSEkRRSADIFI |
| 59 | i1 | 16 | TVF(Aib)SSEkRRSADIFI |
| 60 | il | 16 | TVFRS(Aib)EkRRSADIFI |
| 61 | i1 | 16 | TVFRSSE(Aib)RRSADIFI |
| 62 | i1 | 16 | TVFRSSEk(Aib)RSADIFI |
| 63 | i1 | 16 | TVFRSSEkR(Aib)SADIFI |
| 64 | i1 | 16 | TVFRSSEkRR(Aib)ADIFI |
| 65 | i1 | 16 | TVFRSSEkRRS(Aib)DIFI |
| 66 | i1 | 16 | TVFRSSEkRRSA(Aib)IFI |
| 67 | i1 | 16 | TVFRSSEkRRSADI(Aib)I |
| 68 | i1 | 16 | TVFRSSEkRRSADIF(Aib) |
| 69 | i1 | 16 | GVFRSSEkRRSADIFI |
| 70 | i1 | 16 | TGFRSSEkRRSADIFI |
| 71 | i1 | 16 | TVGRSSEkRRSADIFI |
| 72 | i1 | 16 | TVFGSSEkRRSADIFI |
| 73 | i1 | 16 | TVFRGSEkRRSADIFI |
| 74 | i1 | 16 | TVFRSGEkRRSADIFI |
| 75 | i1 | 16 | TVFRSSGkRRSADIFI |
| 76 | i1 | 16 | TVFRSSEkGRSADIFI |
| 77 | i1 | 16 | TVFRSSEkRGSADIFI |
| 78 | i1 | 16 | TVFRSSEkRRGADIFI |
| 79 | i1 | 16 | TVFRSSEkRRSAGIFI |
| 80 | i1 | 16 | TVFRSSEkRRSADIGI |
| 81 | i1 | 16 | TVFRSSEkRRSADIFG |
| 82 | i1 | 16 | TVDRSSEyRRSADIFI |
| 83 | i1 | 16 | TVDRSSEKRRSADIFI |
| 84 | i1 | 15 | TVFQSEkRRSADQFI |
| 1 | i1 | 17 | TVFRSSREKRRSADIFI |

In another even more specific embodiment, P is selected from the group consisting of SEQ ID NOS: 2, 21, 22, 25, 69, and 71 as listed in Table 2b below:

TABLE 2b

| APJ | SEQ | |
|---|---|---|
| i-Loop | ID NO: | Sequence |
| i1 | 2 | TVFRSSEkRRSADIFI |
| i1 | 22 | TQFRSSEkRRSADIFI |
| i1 | 21 | QVFRSSEkRRSADIFI |
| i1 | 25 | TVFRSQEkRRSADIFI |
| i1 | 69 | GVFRSSEkRRSADIFI |
| i1 | 71 | TVGRSSEkRRSADIFI |

It is understood that the i1 loop sequences, including those sequences presented in Tables 2a and 2b, can be optionally functionalized at the C-terminus by —R₁ of Formulas A and B wherein R₁ is —OR₂ or —NR₃R₄, each R₂ is independently hydrogen or (C₁-C₁₀)alkyl; and
R₃ and R₄ are each independently selected from hydrogen, (C₁-C₁₀) alkyl, (C₁-C₁₀)aralkyl, [CH₂CH₂O]ₙCH₂CH₂C(O)OR₂ or [CH₂CH₂O]ₙCH₂CH₂C(O)NR₂;
n is 1-20
or —NR₃R₄ is a non-aromatic nitrogen-containing heterocyclic group, wherein the non-aromatic group is optionally mono-or di-substituted at one or more substitutable carbon atoms with an R₅;
each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂;

In another specific embodiment, P of Formula I is selected from the group consisting of SEQ ID NO: 1-84, for example those as listed in Tables 2a-2c, and the functionalization at the C-terminus is C(O)NR₃¹R₄¹ (i.e., instead of the C-terminus ending with —C(O)OH, it ends with C(O)NR₃¹R₄¹); R₃¹ and R₄¹ are each independently selected from (C₁-C₁₀)aralkyl, [CH₂CH₂O]ₙCH₂CH₂C(O)OR₂ or [CH₂CH₂O]ₙCH₂CH₂C(O)NR₂;

n is 1-20

or —NR₃¹R₄¹ is a non-aromatic nitrogen-containing heterocyclic group, wherein the non-aromatic group is optionally mono-or di-substituted at one or more substitutable carbon atoms with an R₅;

each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂.

TABLE 2c

| SEQ | | | |
|---|---|---|---|
| ID | Loop | Length | Sequence |
| 1 | i1 | 17 | TVFRSSREKRRSADIFI |

It is understood that as long as P comprises the indicated number of contiguous amino acids residues from the apelin (APJ) intracellular i1 loop from which it is derived, the remainder of the peptide, if present, can be selected from:

(a) any natural amino acid residue, unnatural amino acid residue or a combination thereof;

(b) a peptide sequence comprising natural amino acid residues, non-natural amino acid residues and combinations thereof;

(c) a peptide sequence according to (b) comprising one or more peptide backbone modifications;

(d) a peptide sequence according to (c) comprising one or more retro-inverso peptide linkages;

(e) a peptide sequence according to (c) wherein one or more peptide bonds are replaced by

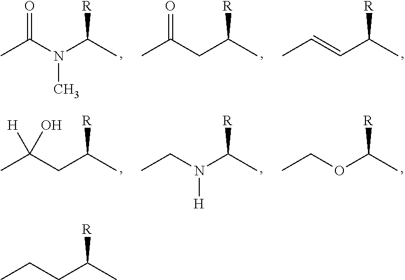

or a combination thereof;

(f) a peptide sequence according to (c) comprising one or more depsipeptide linkages, wherein the amide linkage is replaced with an ester linkage; and

(g) a peptide sequence according to (c) comprising one or more conformational restrictions; and

(h) a peptide sequence according to (c) comprising one or more of (d)-(g). The length of the peptide sequence P can be from 3 amino acids in length to 90 amino acids in length. For example, the length of P is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 67, 68, 69, 70, 71, 72, 73, 74, 75, 76, 77, 78, 79, 80, 81, 82, 83, 84, 85, 86, 87, 88, 89, or 90 amino acids in length.

Furthermore, it is understood that even within the indicated number of contiguous amino acid residues derived from the GPCR intracellular i1 loop, there can be: peptide backbone modifications such as, but not limited to, those described in (e) above; retro-inverso peptide linkages; despsipeptide linkages; conformational restrictions; or a combination thereof.

It is noted that P of Formula I can be optionally functionalized at the C-terminus Functionalized at the C-terminus means that the acid moiety present at the C-terminus is replaced by some other functional group as described herein.

Peptidomimetic as used herein refers to a compound comprising non-peptidic structural elements in place of a peptide sequence.

As used herein, the term “amino acid” includes both a naturally occurring amino acid and a non-natural amino acid.

As used herein, the term “naturally occurring amino acid” means a compound represented by the formula NH₂—CHR—COOH, wherein R is the side chain of a naturally occurring amino acids such as lysine, arginine, serine, tyrosine etc. as shown in the Table below.

Table of Common Naturally Occurring Amino Acids

| | Amino acid | Three letter code | One letter code |
|---|---|---|---|
| Non-polar; | alanine | Ala | A |
| neutral at | isoleucine | Ile | I |
| pH 7.4 | leucine | Leu | L |
| | methionine | Met | M |
| | phenylalanine | Phe | F |
| | proline | Pro | P |
| | tryptophan | Trp | W |
| | valine | Val | V |
| Polar, | asparagine | Asn | N |
| uncharged | cysteine | Cys | C |
| at pH 7.0 | glycine | Gly | G |
| | glutamine | Gln | Q |
| | serine | Ser | S |
| | threonine | Thr | T |
| | tyrosine | Tyr | Y |
| Polar; | glutamic acid | Glu | E |
| charged at | arginine | Arg | R |
| pH 7 | aspartic acid | Asp | D |
| | histidine | His | H |
| | lysine | Lys | K |

“Non-natural amino acid” means an amino acid for which there is no nucleic acid codon. Examples of non-natural amino acids include, for example, the D-isomers of the natural α-amino acids such as D-proline (D-P, D-Pro) as indicated above; natural α-amino acids with non-natural side chains (e.g.,

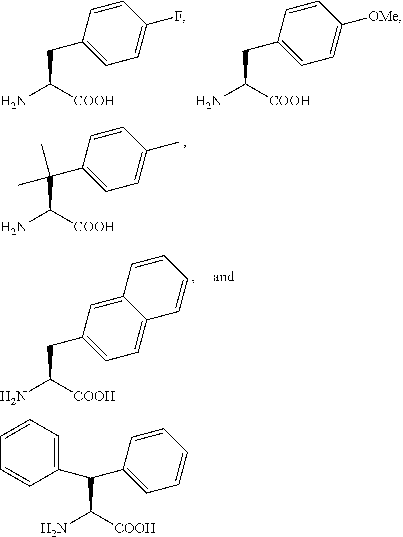

related to phenylalanine); Aib (aminobutyric acid), bAib (3-aminoisobutyric acid), Nva (norvaline), β-Ala, Aad (2-aminoadipic acid), bAad (3-aminoadipic acid), Abu (2-aminobutyric acid), Gaba (γ-aminobutyric acid), Acp (6-aminocaproic acid), Dbu (2,4-diaminobutryic acid), α-aminopimelic acid, TMSA (trimethylsilyl-Ala), aIle (allo-isoleucine), Nle (norleucine), tert-Leu, Cit (citrulline), Om (ornithine, O), Dpm (2,2′-diaminopimelic acid), Dpr (2,3-diaminopropionic acid), α or β—Nal, Cha (cyclohexyl-Ala), hydroxyproline, Sar (sarcosine), Dap (2,3-diaminopropionic acid) and the like.

Unnatural amino acids also include cyclic amino acids; and amino acid analogs, for example, N^{α}-alkylated amino acids such as MeGly (N^{α}-methylglycine), EtGly (N^{α}-ethylglycine) and EtAsn (N^{α}-ethylasparagine); and amino acids in which the α-carbon bears two side-chain substituents. As with the natural amino acids, the residues of the unnatural amino acids are what are left behind when the unnatural amino acid becomes part of a peptide sequence as described herein.

Amino acid residues are amino acid structures as described above that lack a hydrogen atom of the amino group or the hydroxyl moiety of the carboxyl group or both resulting in the units of a peptide chain being amino-acid residues.

The D-isomers of the natural amino acids are designated herein with a lower case letter of the corresponding naturally occurring amino acid. For example, d-proline is designated “p” rather than “P” as is used for naturally occurring proline.

As used herein with respect to Formulas A and B, at least three of X₁-X₁₆ or X₁′-X₁₆ being present and contiguous mean that at least three amino acides that are sequential in number are present. For example, X₁-X₂-X₃ would be contiguous, whereas X₁-X₃-X₄ would not be contiguous. In addition, it is understood that at least 3 of X₁-X₁₆ or X₁′-X₁₆ includes 3, 4, 5, 6, 7, 8, 9, 10, 11, 12, 13, 14, 15 and 16 amino acids.

### Linkers (L)

The linker “L” of the invention connects the lipophilic tether moiety, T, to the N-terminal nitrogen of the N-terminal amino acid residue of P in the case of Formula I and to X₁ and X₁′ or the next present amino acid if X₁ or X₁′ is absent at the atom of L as marked herein with as asterisk in the case of Formulas A and B. The linker can be linear or branched and optionally substituted. The linker can in some instance be used to vary the distance between T and P or the amino acid of Formulas A and B to which it is attached providing a more desirable interaction of P with its cognate GPCR. In other instances, the linker can confer improvements on the physicochemical and pharmacological properties of the APJ receptor compound as compared with compounds lacking a linker. For example, the introduction of the linker can alter one or more of lipophilicity, solubility, partition coefficient, stability, and biological half life.

In one embodiment, wherein L is a linking moiety bonded to the N terminal nitrogen of X₁ of Formula A or X₁′ of Formula B or the next present amino acid residue if X₁ or X₁′ is absent and is selected from: C*(O), C*(S), S*(O)₂, N(R¹³)S*(O), N(R¹³)S*(O)₂, N(R¹³)C*(O), N(R¹³)C*(S), OC*(O), OC*(S), SC*(O), SC*(S), C*(═NH), and N(R¹³)C*(═NH); wherein L is bonded to X₁ or the next present amino acid residue if X₁ is absent at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted.

In another specific embodiment, R³ is H or D.

In a further specific embodiment, L is selected from the group: C*(O), S*(O)₂, NHC*(O) and NHC*(S).

Linkers can be attached to the N-terminal nitrogen of the N-terminal amino acid residue of P using chemistries that are compatible with covalent linkage to nitrogen, including, but not limited to, alkylation, amide bond, urea, thiourea, carbamate, and sulfonamide formation.

### Tethers (T)

T of Formula A, Formula B, or Formula I is a lipohilic tether moiety which imparts lipophilicity to the APJ receptor compounds of the invention. The lipophilicity which T imparts, can promote penetration of the APJ receptor compounds into the cell membrane and tethering of the APJ receptor compounds to the cell membrane. As such, the lipophilicity imparted by T can facilitate interaction between the APJ receptor compounds of the invention and the cognate receptor.

The relative lipophilicity of compounds suitable for use as the lipophilic tether moiety of Formula A, Formula B, or Formula I can be quantified by measuring the amount of the compound that partitions into an organic solvent layer (membrane-like) vs. an aqueous solvent layer (analogous to the extracellular or cytoplasmic environment). The partition coefficient in a mixed solvent composition, such as octanol/water or octanol/PBS, is the ratio of compound found at equilibrium in the octanol vs. the aqueous solvent (Partition coeff P=[compound]_{octanol}/[compound]_{aqueous}). Frequently, the partition coefficient is expressed in logarithmic form, as the log P. Compounds with greater lipophilicity have a more positive log P than more hydrophilic compounds and tend to interact more strongly with membrane bilayers.

Computational programs are also available for calculating the partition coefficient for compounds suitable for use as the lipophilic tether moiety (T). In situations where the chemical structure is being varied in a systematic manner, for example by adding additional methylene units (—CH₂—) onto to an existing alkyl group, the trend in log P can be calculated using, for example, ChemDraw (CambridgeSoft, Inc).

In one embodiment, T is an optionally substituted (C₆-C₃₀)alkyl, (C₆-C₃₀)alkenyl, (C₆-C₃₀)alkynyl wherein 0-3 carbon atoms are replaced with oxygen, sulfur, nitrogen or a combination thereof.

In a specific embodiment, the (C₆-C₃₀)alkyl, (C₆-C₃₀)alkenyl, (C₆-C₃₀)alkynyl are substituted at one or more substitutable carbon atoms with halogen, —CN, —OH, —NH₂, NO₂, —NH(C₁-C₆)alkyl, —N((C₁-C₆)alkyl)₂, (C₁-C₆)alkyl, (C₁-C₆)haloalkyl, (C₁-C₆)alkoxy, (C₁-C₆)haloalkoxy, aryloxy, (C₁-C₆)alkoxycarbonyl, —CONH₂, —OCONH₂, —NHCONH₂, C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H; and p is 1 or 2.

In a specific embodiment, T is selected from the group consisting of: CH₃(CH₂)₉OPh-, CH₃(CH₂)₆C═C(CH₂)₆, CH₃(CH₂)₁₁O(CH₂)₃, CH₃(CH₂)₉O(CH₂)₂ and CH₃(CH₂)₁₃.

In a specific embodiment, T is selected from the group consisting of: CH₃(CH₂)₁₆, CH₃(CH₂)₁₅, CH₃(CH₂)₁₄, CH₃(CH₂)₁₃, CH₃(CH₂)₁₂, CH₃(CH₂)₁ᵢ, CH₃(CH₂)₁₀, CH₃(CH₂)₉, CH₃(CH₂)₈, CH₃(CH₂)₉OPh-, CH₃(CH₂)₆C═C(CH₂)₆, CH₃(CH₂)₁₁O(CH₂)₃, and CH₃(CH₂)₉O(CH₂)₂ and CH₃(CH₂)₁₃.

It is understood that the lipophilic moiety (T) of Formula A, Formula B, or Formula I can be derived from precursor liphophilic compounds (e.g., fatty acids and bile acids). As used herein, “derived from” with regard to T, means that T is derived from a precursor lipophilic compound and that reaction of the precursor lipophilic compound in preparing the APJ receptor compounds of Formula A, Formula B, or Formula I, results in a lipophilic tether moiety represented by T in Formula A, Formula B, or Formula I that is structurally modified in comparison to the precursor lipophilic compound.

For example, the lipophilic tether moiety, T of Formula A, Formula B, or Formula I can be derived from a fatty acid or a bile acid. It is understood that in accordance with Formula A, Formula B, or Formula I, when T is derived from a fatty acid (i.e., a fatty acid derivative), it is attached to L-P at the carbon atom alpha to the carbonyl carbon of the acid functional group in the fatty acid from which it is derived. For example, when T is derived from palmitic acid,

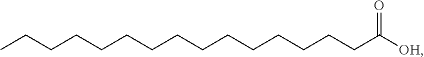 [SMILES: CCCCCCCCCCCCCCCC(=O)O]

T of Formula A, Formula B, or Formula I has the following structure:

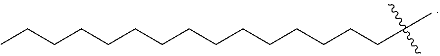 [SMILES: *CCCCCCCCCCCCCCC]

Similarly, when T is derived from stearic acid,

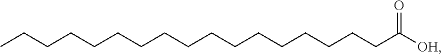 [SMILES: CCCCCCCCCCCCCCCCCC(=O)O]

T of Formula A, Formula B, or Formula I has the following structure:

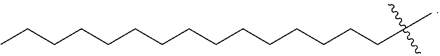 [SMILES: *CCCCCCCCCCCCCCC]

Similarly, when T is derived from 3-(dodecyloxy)propanoic acid,

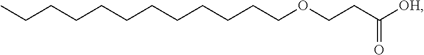 [SMILES: CCCCCCCCCCCCOCCC(=O)O]

T of Formula A, Formula B, or Formula I has the following structure:

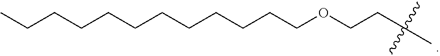 [SMILES: *CCOCCCCCCCCCCCC]

Similarly, when T is derived from 4-(undecyloxy)butanoic acid,

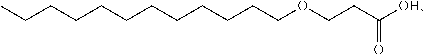 [SMILES: CCCCCCCCCCCCOCCC(=O)O]

T of Formula A, Formula B, or Formula I has the following structure:

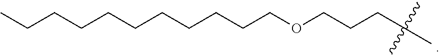 [SMILES: *CCCOCCCCCCCCCCC]

Similarly, when T is derived from elaidic acid,

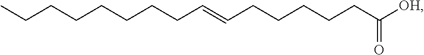 [SMILES: CCCCCCCC/C=C/CCCCCC(=O)O]

T of Formula A, Formula B, or Formula I has the following structure:

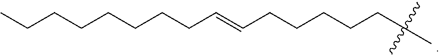 [SMILES: *CCCCC/C=C/CCCCCCCC]

Similarly when T is derived from oleic acid

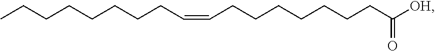 [SMILES: CCCCCCCC/C=C\CCCCCCCC(=O)O]

T of Formula A, Formula B, or Formula I has the following structure:

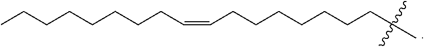 [SMILES: *CCCCCCC/C=C\CCCCCCCC]

Similarly, when T is derived from 16-hydroxypalmitic acid,

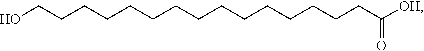 [SMILES: O=C(O)CCCCCCCCCCCCCCCO]

T of Formula A, Formula B, or Formula I has the following structure:

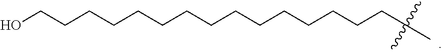 [SMILES: *CCCCCCCCCCCCCCCO]

Similarly, when T is derived from 2-aminooctadecanoic acid

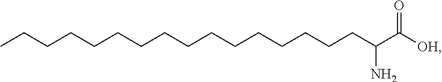 [SMILES: CCCCCCCCCCCCCCCCC(N)C(=O)O]

T of Formula A, Formula B, or Formula I has the following structure:

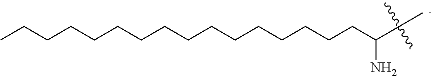 [SMILES: *C(N)CCCCCCCCCCCCCCCC]

Similarly, when T is derived from 2-amino-4-(dodecyloxy)butanoic acid,

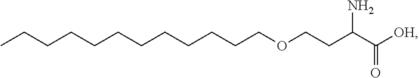 [SMILES: CCCCCCCCCCCCOCCC(N)C(=O)O]

T of Formula A, Formula B, or Formula I has the following structure:

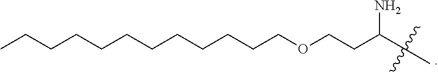 [SMILES: *C(N)CCOCCCCCCCCCCCC]

In a further embodiment, T is derived from a fatty acid. In a specific embodiment, T is derived from a fatty acid selected from the group consisting of: butyric acid, caproic acid, caprylic acid, capric acid, lauric acid, myristic acid, palmitic acid, stearic acid, arachidic acid, behenic acid, and lignoceric acid.

In another specific embodiment, T is derived from a fatty acid selected from the group consisting of: myristoleic acid, palmitoleic acid, oleic acid, linoleic acid, α-linolenic acid, arachidonic acid, eicosapentaenoic acid, erucic acid, docosahexaenoic acid

In another embodiment, T of Formula A, Formula B, or Formula I can be derived from a bile acid. Similar to the embodiment where T is a fatty acid derivative, it is understood that in accordance with Formula A, Formula B, or Formula I, when T is derived from a bile acid (i.e., a bile acid derivative) it is attached to L-P at the carbon atom alpha to the carbonyl carbon of the acid functional group in the bile acid from which it is derived. For example, when T is derived from lithocholic acid,

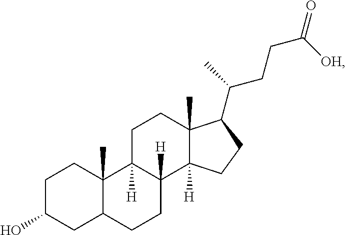

T of Formula A, Formula B, or Formula I has the following structure:

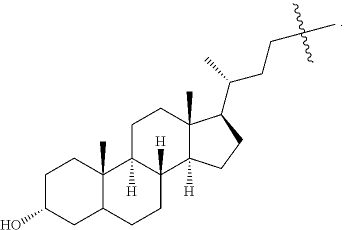

In a further embodiment, T is derived from a bile acid. In a specific embodiment, T is derived from a bile acid selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, hyodeoxycholic acid and the like.

For example, T is selected from:

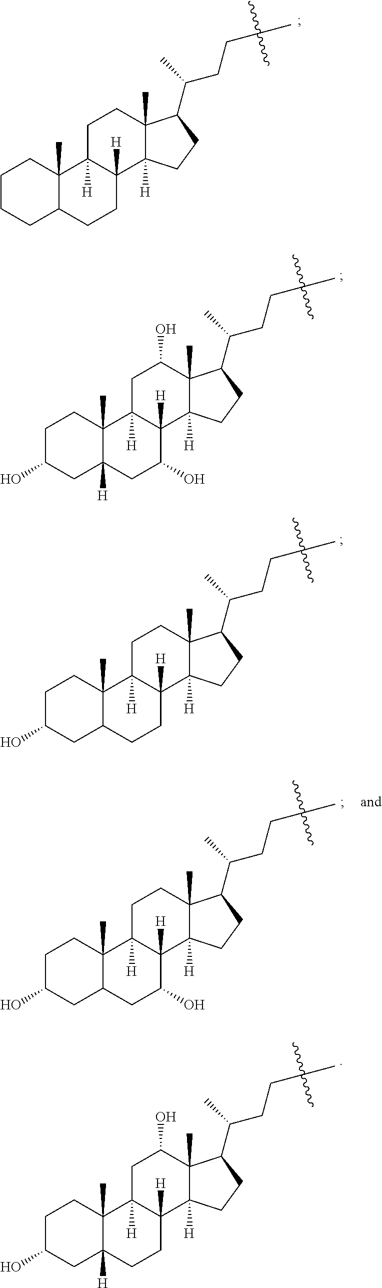

In another further embodiment, T is derived from a bile acid described above that has been modified at other than the acid functional group. For example, T can be derived from any of the bile acids described above, where the hydroxy position has been modified to form an ester or a halo ester. For example, T can be:

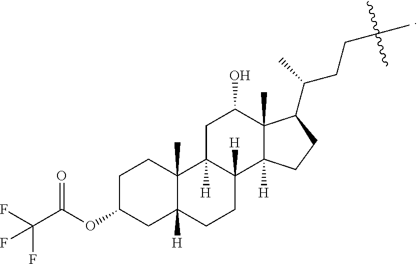

Other lipophilic moieties suitable for use as the lipophilic membrane tether, T, of Formula A, Formula B, or Formula I, include but are not limited to steroids. Suitable steroids include, but are not limited to, sterols; progestagens; glucocorticoids; mineralcorticoids; androgens; and estrogens. Generally any steroid capable of attachment or which can be modified for incorporation into Formula A, Formula B, or Formula I can be used. It is understood that the lipophilic membrane tether, T, may be slightly modified from the precursor lipophilic compound as a result of incorporation into Formula A, Formula B, or Formula I.

Suitable sterols for use in the invention at T, include but are not limited to: cholestanol, coprostanol, cholesterol, epicholesterol, ergosterol, ergocalciferol, and the like. Preferred sterols are those that provide a balance of lipophilicity with water solubility.

Suitable progestagens include, but are not limited to progesterone. Suitable glucocorticoids include, but are not limited to cortisol. Suitable mineralcorticoids include, but are not limited to aldosterone. Suitable androgens include, but are not limited to testosterone and androstenedione. Suitable estrogens include, but are not limited to estrone and estradiol.

In another specific embodiment, T can be derived from 2-tetradecanamideooctadecanoid acid. Similar to the embodiment where T is a fatty acid derivative, it is understood that in accordance with Formula A, Formula B, or Formula I, when T is derived from 2-tetradecanamideooctadecanoid acid it is attached to L-P at the carbon atom alpha to the carbonyl carbon of the acid functional group in the bile acid from which it is derived. For example, when T is derived from 2-tetradecanamideooctadecanoid acid, the tether is:

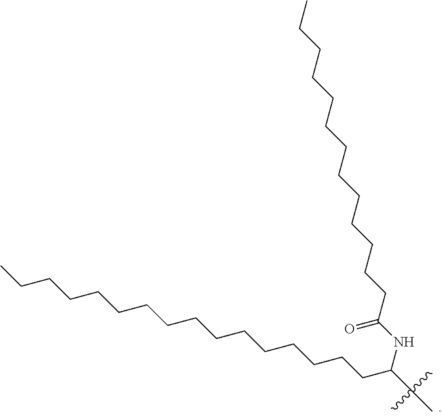 [SMILES: *C(CCCCCCCCCCCCCCCC)NC(=O)CCCCCCCCCCCCC]

In another embodiment, T of Formula A, Formula B, or Formula I can be derived from 2-(5-((3aS,4S,6aR)-2-oxohexahydro-1H-thieno[3,4-d]imidazol-4-yepentanamido)octadecanoic acid. For example, when T is derived from 2-(5-((3aS,4S,6aR)-2-oxohexahydro-1H-thieno[3,4-d]imidazol-4-yl)pentanamido)octadecanoic acid, the tether is:

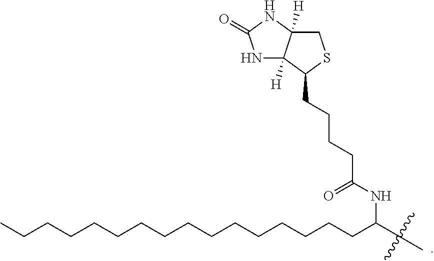 [SMILES: *C(CCCCCCCCCCCCCCCC)NC(=O)CCCC[C@@H]1SC[C@@H]2NC(=O)N[C@@H]21]

In yet another embodiment, T of Formula A, Formula B, or Formula I can be:

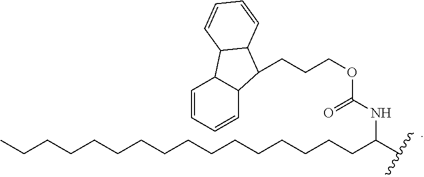 [SMILES: *C(CCCCCCCCCCCCCCCC)NC(=O)OCCCC1C2C=CC=CC2C2C=CC=CC21]

It is understood, that the compounds can contain one of more tether moieties. In certain aspects, the tether moieties are the same. In other embodiments, the tether moieties are different.

### Compounds (T-L-P)

In a first aspect of the invention, the APJ compounds of the invention are represented by Formula A:

T-L-X₁-X₂-X₃-X₄-X₅-X₆-X₇-X₈-X₉-X₁₀-X₁₁-X₁₂-X₁₃-X₁₄-X₁₅-X₁₆-R₁;

or a pharmaceutically acceptable salt thereof, wherein L is a linking moiety bonded to the N terminal nitrogen of X₁ or the next present amino acid residue if X₁ is absent and is selected from: C*(O), C*(S), S*(O)₂, N(R¹³)S*(O), N(R¹³)S*(O)₂, N(R¹³)C*(O); N(R¹³)C*(S), OC*(O), OC*(S), SC*(O), SC*(S), C*(═NH), and N(R¹³)C*(═NH); wherein L is bonded to X₁ or the next present amino acid residue if X₁ is absent at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; T is a lipophilic moiety bonded to L; R₁ is —OR₂—, or NR₃R₄; wherein

each R₂ is hydrogen or a (C₁-C₁₀) alkyl group,

R₃ and R₄ are each independently selected from hydrogen, (C₁-C₁₀) alkyl, (C₁-C₁₀)aralkyl, [CH₂CH₂O]ₙCH₂CH₂C(O)OR₂ or [CH₂CH₂O]ₙCH₂CH₂C(O)NR₂;

n is 1-20

or —NR₃R₄ is a non-aromatic nitrogen-containing heterocyclic group, wherein the non-aromatic group is optionally mono-or di-substituted at one or more substitutable carbon atoms with an R₅;

each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂X₁ is absent, a threonine residue, a histidine residue, a glutamine residue, an aspartic acid residue, an Aib residue, a phenylalanine residue, or a glycine residue;
X₂ is absent, a valine residue, a phenylalanine residue, a histidine residue, an Aib residue, a glutamine residue, an aspartic acid residue, a proline residue, or a glycine residue;
X₃ is absent, a phenylalanine residue, a histidine residue, an aspartic acid residue, a glycine residue, or a proline residue;
X₄ is absent, an arginine residue, a phenylalanine residue, a histidine residue, a glutamine residue, an aspartic acid residue, an Aib residue, a proline residue, or a glycine residue;
X₅ is absent, a serine residue, a phenylalanine residue, a histidine residue, a glutamine residue, an aspartic acid residue, a proline residue, an Aib residue, or a glycine residue;
X₆ is absent, a serine residue, a phenylalanine residue, a histidine residue, a glutamine residue, an aspartic acid residue, a proline residue, an Aib residue, or a glycine residue;
X₇ is absent, a glutamic acid, a phenylalanine residue, a histidine residue, a glutamine residue, an aspartic acid residue, a proline residue, an Aib residue, or a glycine residue;
X₈ is absent, a lysine, D-lysine residue, a phenylalanine residue, a proline residue, a glutamic acid, a phenylalanine residue, a histidine residue, a glutamine residue, an aspartic acid residue, an Aib residue, or a glycine residue, or a tyrosine residue;
X₉ is absent, an arginine residue, a phenylalanine residue, a histidine residue, a glutamine residue, an aspartic acid residue, a proline residue, an Aib residue, or a glycine residue;
X₁₀ is absent, an arginine residue, a phenylalanine residue, a histidine residue, a glutamine residue, an aspartic acid residue, a proline residue, an Aib residue, or a glycine residue;
X₁₁ is absent, a serine residue, a phenylalanine residue, a histidine residue, a glutamine residue, an aspartic acid residue, a proline residue, an Aib residue, or a glycine residue;
X₁₂ is absent or an alanine residue, a phenylalanine residue, a histidine residue, a glutamine residue, an aspartic acid residue, a proline residue, an Aib residue, or a glycine residue;
X₁₃ is absent or an aspartic acid residue, a phenylalanine residue, a histidine residue, a glutamine residue, an aspartic acid residue, a proline residue, an Aib residue, or a glycine residue;
X₁₄ is absent or an isoleucine residue, a phenylalanine residue, a histidine residue, a glutamine residue, an aspartic acid residue, a proline residue, an Aib residue, or a glycine residue;
X₁₅ is absent, a phenylalanine residue, a histidine residue, a glutamine residue, an aspartic acid residue, a proline residue, an Aib residue, or a glycine residue;
X₁₆ is absent, a phenylalanine residue, an isoleucine residue, a phenylalanine residue, a histidine residue, a glutamine residue, an aspartic acid residue, a proline residue, an Aib residue, or a glycine residue;
wherein at least three of X₁-X₁₆ are present and contiguous and optionally 1-5 amino acid residues are present in the D configuration.

In a specific embodiment of the first aspect, X₁ is absent, a threonine residue, a glutamine residue, an aspartic acid residue, a glycine residue;
X₂ is absent, a valine residue, an Aib residue, a glutamine residue, or a glycine residue;
X₃ is absent, a phenylalanine residue, a glycine residue, an aspartic acid residue or a histidine residue;
X₄ is absent, an arginine residue, an Aib residue, a proline residue, or a glycine residue;
X₅ is absent, a serine residue or a glycine residue;
X₆ is absent, a serine residue, or a histidine residue;
X₇ is absent, a glutamic acid residue, or a proline residue;
X₈ is D-lysine residue or a proline residue;
X₉ is absent, an arginine residue, phenylalanine residue, an Aib residue, or a glutamine residue;
X₁₀ is absent, an arginine residue or a histidine residue;
X₁₁ is absent, a serine residue or an aspartic acid residue;
X₁₂ is absent or an alanine residue;
X₁₃ is absent or an aspartic acid residue;
X₁₄ is absent or an isoleucine residue, a glutamine residue or an aspartic acid residue;
X₁₅ is absent or a phenylalanine residue, or an Aib residue; and
X₁₆ is absent or an isoleucine residue, a phenylalanine residue, or a proline residue.

In another specific embodiment of the first aspect of the invention, at least nine amino acids of X₁-X₁₆ are present or all of X₁-X₁₆ are present. In a more specific embodiment of the first aspect, X₈ is D-lysine. In another specific embodiment of the first aspect of the invention, X₁ is a glutamine residue, an aspartic acid residue, or a glycine residue. In another specific embodiment of the first aspect of the invention, X₃ is a glycine residue or an aspartic acid residue. In another specific embodiment of the first aspect of the invention, X₄ is a proline residue or a glycine residue. In another specific embodiment of the first aspect of the invention, X₆ is a glutamine residue. In another specific embodiment of the first aspect of the invention, X₉ is a phenylalanine residue, a glutamine residue, or an Aib residue. In another specific embodiment of the first aspect of the invention, X₁₀ is a histidine residue. In another specific embodiment of the first aspect of the invention, X₁₅ is an Aib residue;

In another embodiment of the first aspect of the invention, X₁ is a threonine residue, a glutamine residue, or a glycine residue;
X₂ is a valine residue;
X₃ is a phenylalanine residue or a glycine residue;
X₄ is an arginine residue;
X₅ is a serine residue;
X₆ is a serine residue;
X₇ is a glutamic acid residue;
X₈ is D-lysine residue;
X₉ is an arginine residue;
X₁₀ is an arginine residue;
X₁₁ is a serine residue;
X₁₂ is an alanine residue;
X₁₃ is an aspartic acid residue;
X₁₄ is an isoleucine residue;
X₁₅ is a phenylalanine residue; and
X₁₆ is an isoleucine residue.

In another specific embodiment of the first aspect of the invention,
X₁ is a threonine residue or an aspartic acid residue;
X₂ is a valine residue;
X₃ is a phenylalanine residue or an aspartic acid residue;
X₄ is an arginine residue, a proline residue or a glycine residue;
X₅ is a serine residue;
X₆ is a serine residue;
X₇ is a glutamic acid residue;
X₈ is D-lysine residue;
X₉ is an arginine residue a phenylalanine residue, an Aib residue or a glutamine residue;
X₁₀ is an arginine residue or a histidine residue;
X₁₁ is a serine residue;
X₁₂ is an alanine residue;
X₁₃ is an aspartic acid residue;
X₁₄ is an isoleucine residue;
X₁₅ is a phenylalanine residue or an Aib residue; and
X₁₆ is an isoleucine residue.
   In another embodiment of the first aspect of the invention:
X₁ is a threonine residue;
X₂ is a valine residue, a phenylalanine residue, a histadine residue, an Aib residue, a glutamine residue, an aspartic acid residue, a proline residue, or a glycine residue;
X₃ is a phenylalanine residue or a histidine residue;
X₄ is an arginine residue;
X₅ is a serine residue;
X₆ is a serine residue or a histidine residue;
X₇ is a glutamic acid residue or a proline residue;
X₈ is a D-lysine residue;
X₉ is an arginine residue;
X₁₀ is an arginine residue;
X₁₁ is a serine residue;
X₁₂ is an alanine residue;
X₁₃ is an aspartic acid residue;
X₁₄ is an isoleucine residue or a glutamine residue or an aspartic acid residue;
X₁₅ is a phenylalanine residue; and
X₁₆ is an isoleucine residue, a phenylalanine residue or a proline residue.

In a specific embodiment of the first aspect, the compound of the invention selected from any one of Compound Nos. 1-82 and 87. In a more particular embodiment, the compound is selected from compounds: 1, 20, 24, 68 and 70. In another specific embodiment, the compound is selected from Compound Nos. 3, 14, 26, 33, 35, 44, 60, 66, and 71.

In a second aspect of the invention, the compounds are represented by Formula I:

T-L-P,

or pharmaceutically acceptable salts thereof, wherein:

P is a peptide comprising at least three contiguous amino-acid residues of the intracellular i1 loop of the APJ receptor, wherein

L is a linking moiety bonded to P at an N-terminal nitrogen of an N-terminal amino-acid residue selected from: C*(O), C*(S), S*(O)₂, N(R¹³)S*(O), N(R¹³)S*(O)₂, N(R¹³)C*(O), N(R¹³)C*(S), OC*(O), OC*(S), SC*(O), SC*(S), C*(═NH), and N(R¹³)C*(═NH); wherein L is bonded to P at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; and T is a lipophilic tether moiety bonded to L, wherein the C-terminal amino acid residue of P is functionalized by replacement of the acid moiety with C(O)NR₃¹R₄¹; R₃¹ is selected from hydrogen, C₁-C₁₀ alkyl, (C₁-C₁₀)aralkyl, [CH₂CH₂O]ₙCH₂CH₂C(O)OR₂ or [CH₂CH₂O]ₙCH₂CH₂C(O)NR₂; R₄¹ is selected from (C₁-C₁₀)aralkyl, [CH₂CH₂O]ₙCH₂CH₂C(O)OR₂ or [CH₂CH₂O]ₙCH₂CH₂C(O)NR₂;

n is 1-20

or —NR₃¹R₄¹ is a non-aromatic nitrogen-containing heterocyclic group, wherein the non-aromatic group is optionally mono-or di-substituted at one or more substitutable carbon atoms with an R₅;

each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂.

In a specific embodiment of the second aspect, the compounds are selected from Compound Nos: 83, 84, 85 and 86.

In another specific embodiment of the second aspect of the invention P is selected from SEQ ID NO: 1-84.

In a third aspect, T is an optionally substituted (C₆-C₃₀)alkyl, (C₆-C₃₀)alkenyl, (C₆-C₃₀)alkynyl, wherein 0-3 carbon atoms are replaced with oxygen, sulfur, nitrogen or a combination thereof. This value of T is applicable to the first and second aspects and the embodiments and specific (i.e., specific, more specific and most specific) embodiments of same.

In a specific embodiment of the third aspect, T is selected from: CH₃(CH₂)₁₆, CH₃(CH₂)₁₅, CH₃(CH₂)₁₄, CH₃(CH₂)₁₃, CH₃(CH₂)₁₂, CH₃(CH₂)₁₁, CH₃(CH₂)₁₀, CH₃(CH₂)₉, CH₃(CH₂)₈, CH₃(CH₂)₉OPh-, CH₃(CH₂)₆C═C(CH₂)₆, CH₃(CH₂)₁₁O(CH₂)₃, and CH₃(CH₂)₉O(CH₂)₂.

In another specific embodiment of the third aspect, T is a fatty acid derivative.

In a more specific embodiment of the third aspect, the fatty acid is selected from the group consisting of: butyric acid, caproic acid, caprylic acid, capric acid, lauric acid, myristic acid, palmitic acid, stearic acid, arachidic acid, behenic acid, lignoceric acid, myristoleic acid, palmitoleic acid, oleic acid, linoleic acid, α-linolenic acid, arachidonic acid, eicosapentaenoic acid, erucic acid, docosahexaenoic acid.

In a fourth aspect, T is a bile acid derivative. This value of T is applicable to the first, second and third, and the embodiments and specific (i.e., specific, more specific and most specific) embodiments of same.

In a specific embodiment of the fourth aspect, the bile acid is selected from the group consisting of: lithocholic acid, chenodeoxycholic acid, deoxycholic acid, cholanic acid, cholic acid, ursocholic acid, ursodeoxycholic acid, isoursodeoxycholic acid, lagodeoxycholic acid, dehydrocholic acid, hyocholic acid, and hyodeoxycholic acid.

In a fifth aspect, T is selected from sterols; progestagens; glucocorticoids; mineralcorticoids; androgens; and estrogens. This value of T is applicable to the first, second, third and fourth aspects and the embodiments and specific (i.e., specific, more specific and most specific) embodiments of same.

In a sixth aspect, T-L of Formula A, Formula B, or Formula I is represented by a moiety selected from the group consisting of:

CH₃(CH₂)₁₅—C(O);

CH₃(CH₂)₁₃—C(O);

CH₃(CH₂)₉O(CH₂)₂C(O);

CH₃(CH₂)₁₀O(CH₂)₂C(O);

CH₃(CH₂)₆C≡C(CH₂)₆—C(O);

LCA-C(O); and

CH₃(CH₂)₉OPh-C(O) wherein

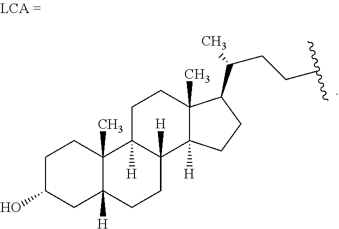

In an seventh aspect, T of Formula A, Formula B, or Formula I is represented by a moiety selected from the group consisting of:

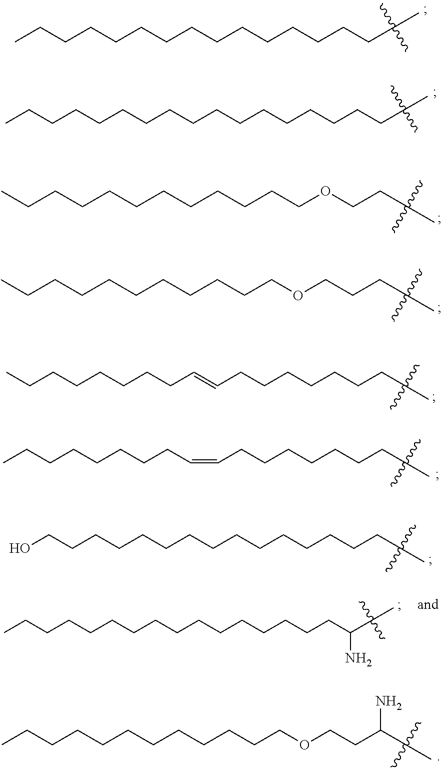

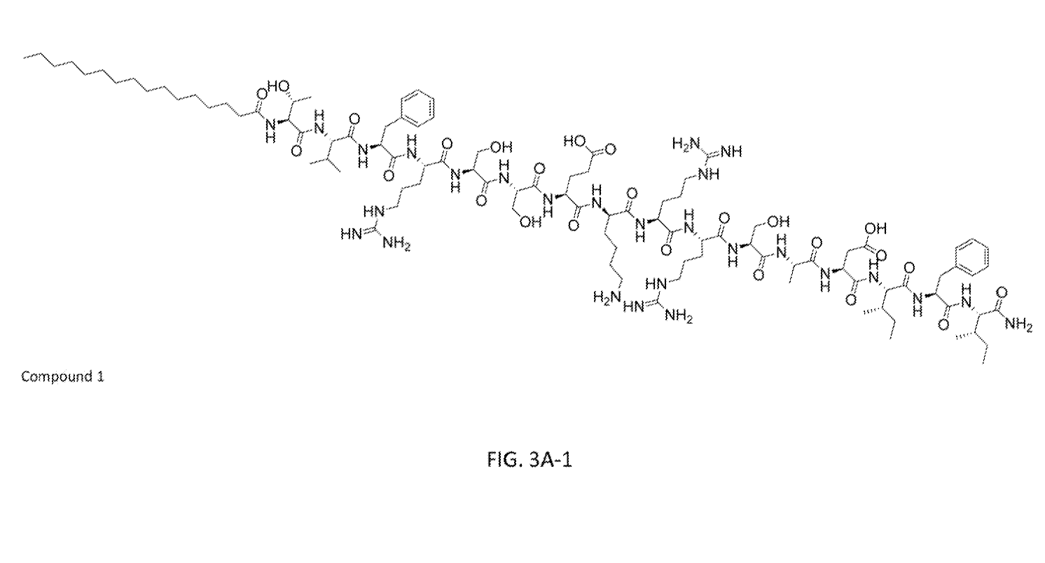

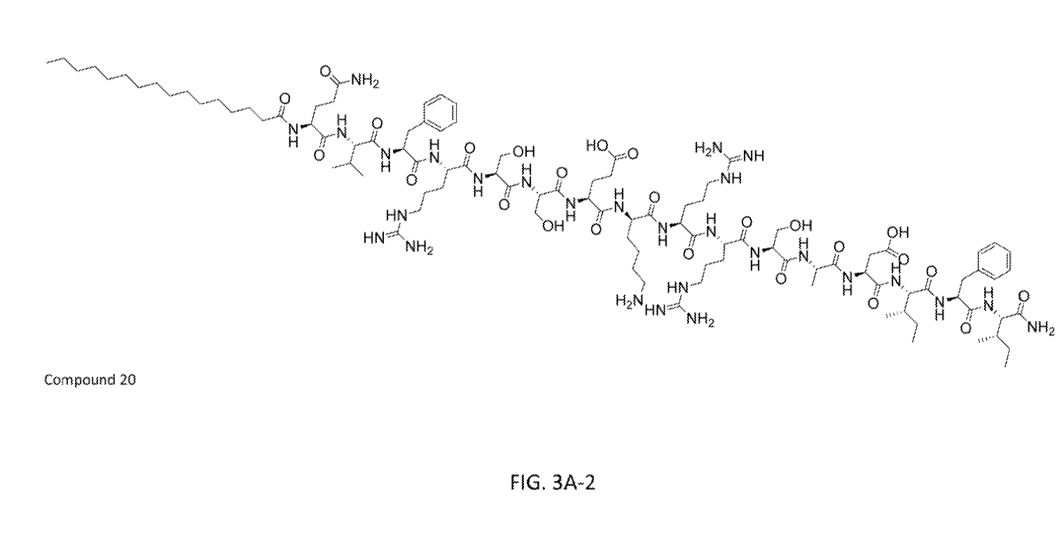

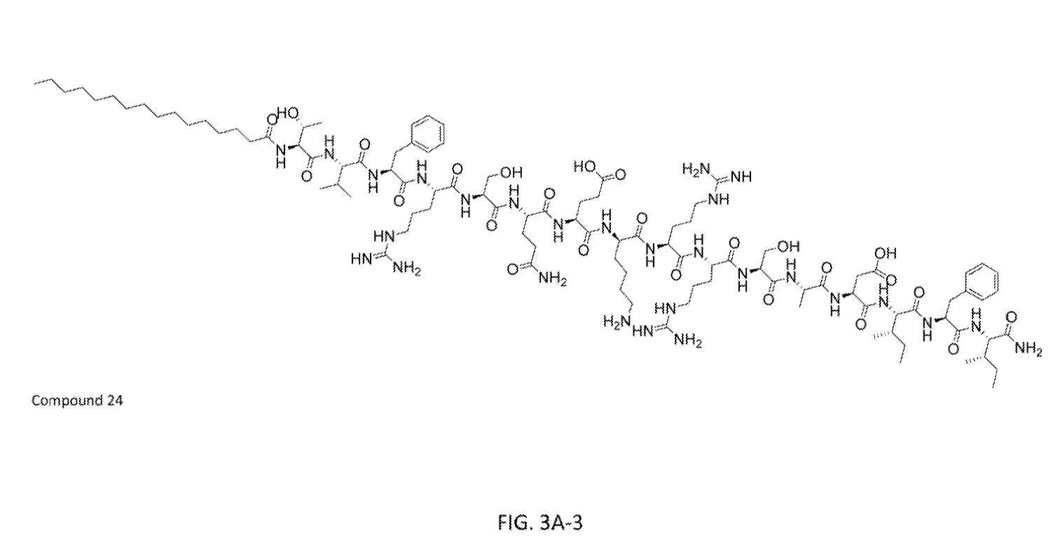

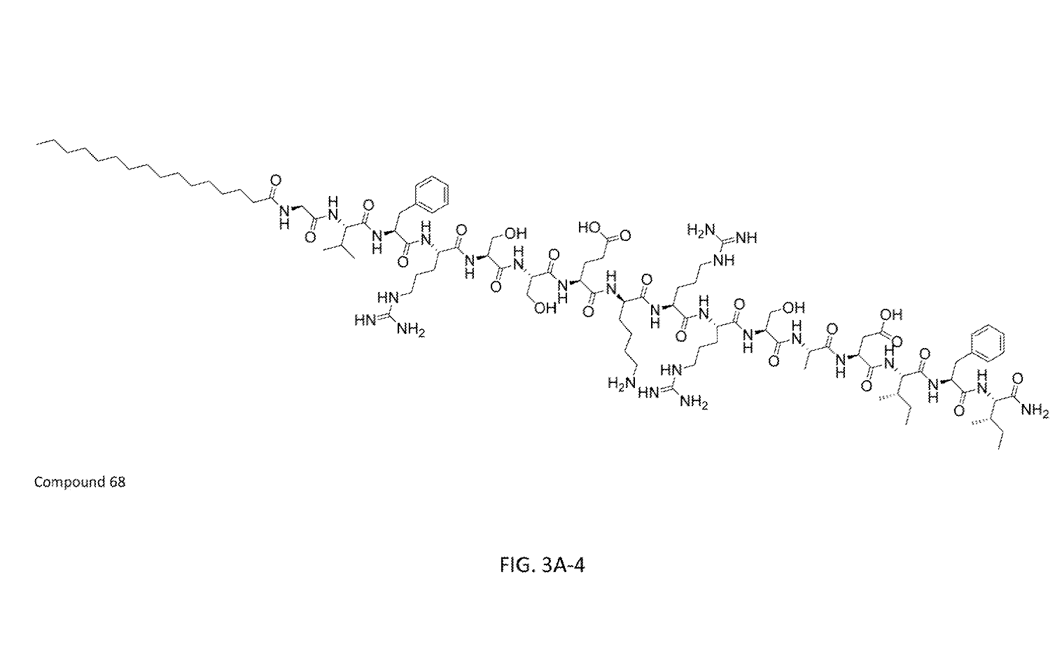

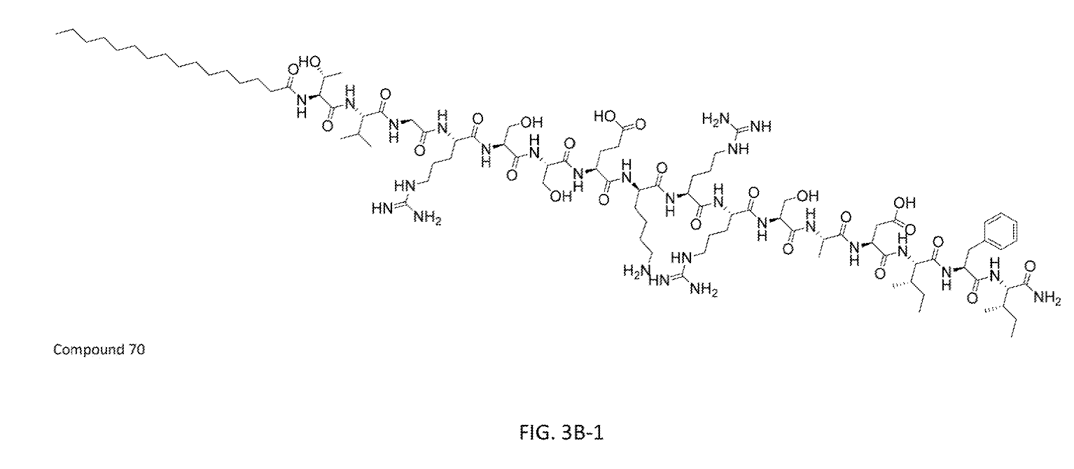

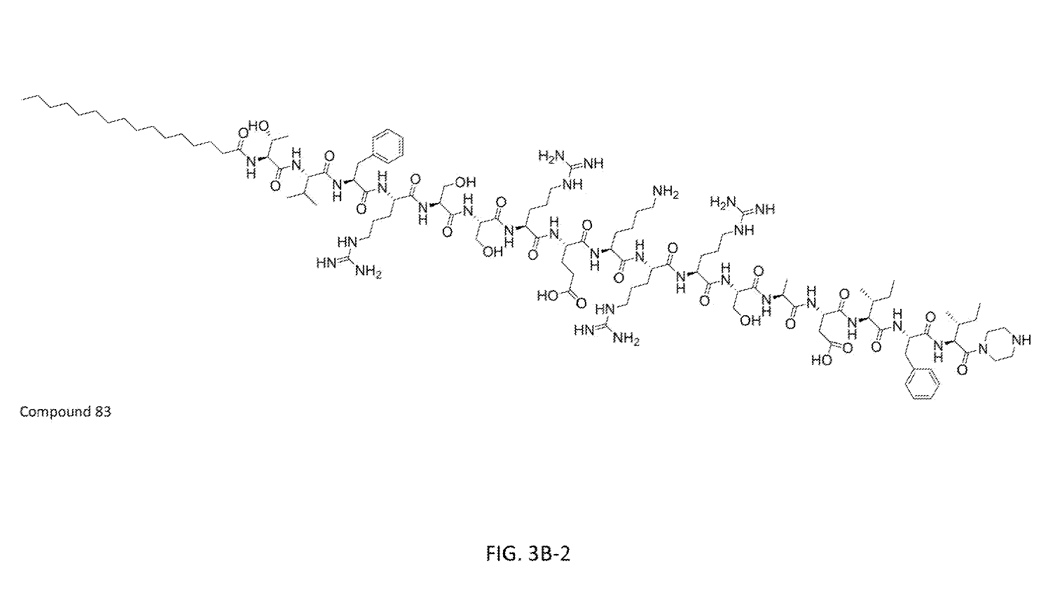

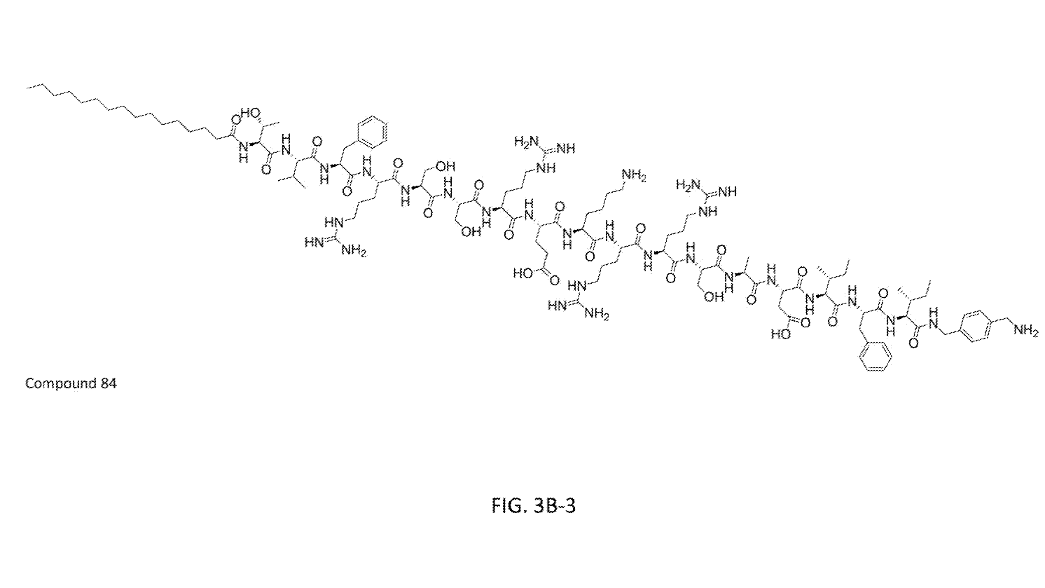

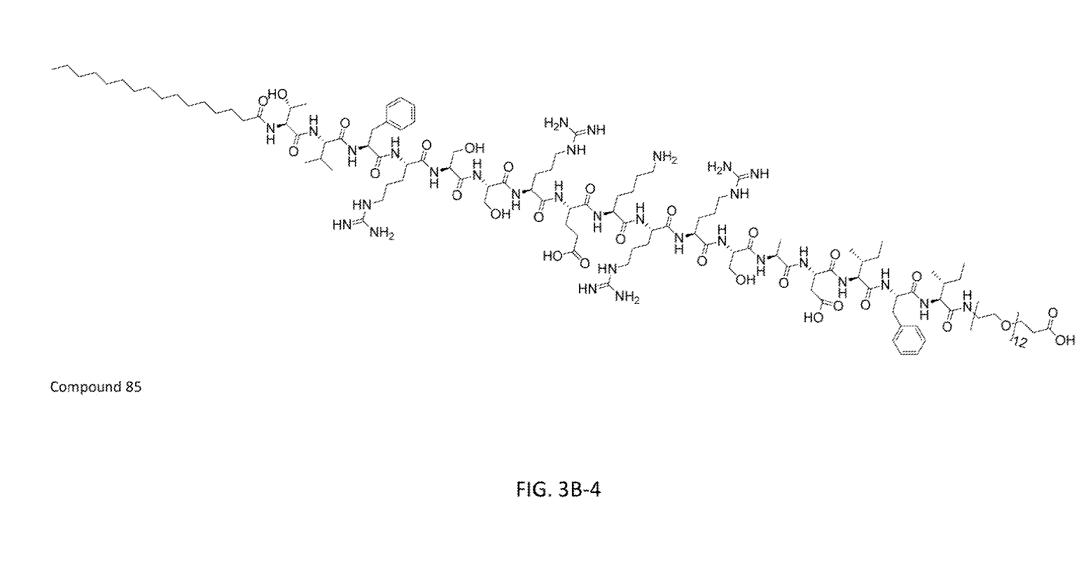

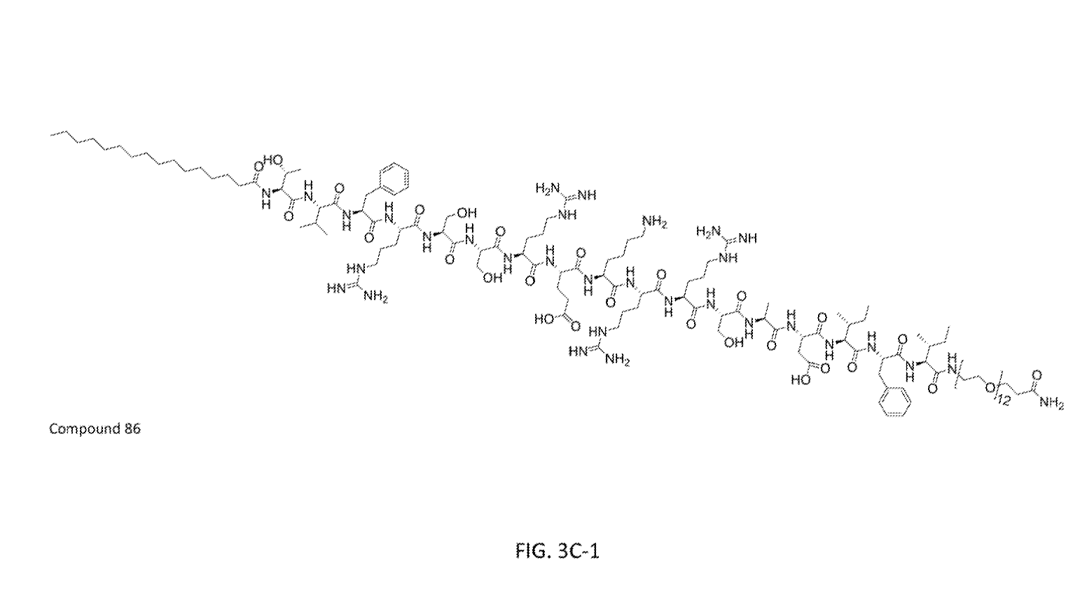

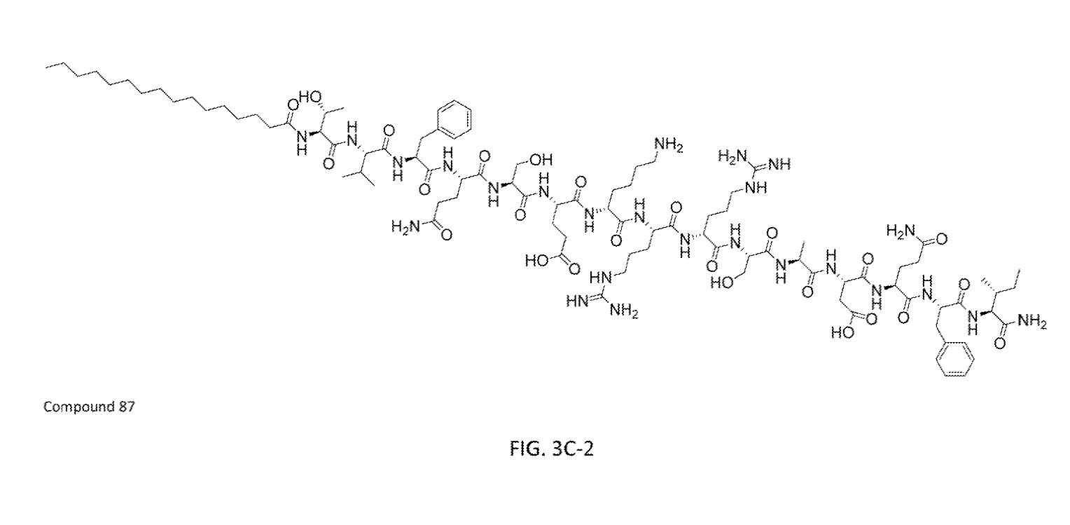

In yet another embodiment, a GPCR compound of the invention is selected from one of the following compounds in Table 3 or shown in FIGS. 3A-3C or a pharmaceutically acceptable salt thereof:

TABLE 3

| Cmpd | | | | |
|---|---|---|---|---|
| # | Tether | Linker | Sequence | C-terminus |
| 1 | Pal | —C(O)— | TVFRSSEkRRSADIFI | NH₂ |
| | | | (SEQ ID NO: 2) | |
| 20 | Pal | —C(O)— | QVFRSSEkRRSADIFI | NH₂ |
| | | | (SEQ ID NO: 21) | |
| 24 | Pal | —C(O)— | TVFRSQEkRRSADIFI | NH₂ |
| | | | (SEQ ID NO: 25) | |
| 68 | Pal | —C(O)— | GVFRSSEkRRSADIFI | NH₂ |
| | | | (SEQ ID NO: 69) | |
| 70 | Pal | —C(O)— | TVGRSSEkRRSADIFI | NH₂ |
| | | | (SEQ ID NO: 71) | |

“Cycloalkyl” used alone or as part of a larger moiety such as “cycloalkylalkyl” refers to a monocyclic or polycyclic, non-aromatic ring system of 3 to 20 carbon atoms, 3 to 12 carbon atoms, or 3 to 9 carbon atoms, which may be saturated or unsaturated. Examples of cycloalkyl groups include cyclopropyl, cyclobutyl, cyclopentyl, cyclohexyl, cyclohexenyl, cyclohexa-1,3-dienyl, cyclooctyl, cycloheptanyl, norbornyl, adamantyl, and the like.

“Heterocycloalkyl” refers to a saturated or unsaturated, non-aromatic, monocyclic or polycyclic ring system of 3 to 20 atoms, 3 to 12 atoms, or 3 to 8 atoms, containing one to four ring heteroatoms chosen from O, N and S. Examples of heterocycloalkyl groups include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine-1,1-dioxide, tetrahydro-2H-1,2-thiazine-1,1-dioxide, isothiazolidine-1,1-dioxide, pyrrolidin-2-one, piperidin-2-one, piperazin-2-one, and morpholin-2-one, and the like.

“Halogen” and “halo” refer to fluoro, chloro, bromo or iodo.

“Haloalkyl” refers to an alkyl group substituted with one or more halogen atoms. By analogy, “haloalkenyl”, “haloalkynyl”, etc., refers to the group (for example alkenyl or alkynyl) substituted by one or more halogen atoms.

“Cyano” refers to the group —CN.

“Oxo” refers to a divalent ═O group.

“Thioxo” refers to a divalent ═S group.

“Ph” refers to a phenyl group.

“Carbonyl” refers to a divalent —C(O)— group.

“Alkyl” used alone or as part of a larger moiety such as “hydroxyalkyl”, “alkoxyalkyl”, “alkylamine” refers to a straight or branched, saturated aliphatic group having the specified number of carbons, typically having 1 to 12 carbon atoms. More particularly, the aliphatic group may have 1 to 10, 1 to 8, 1 to 6, or 1 to 4 carbon atoms. This term is exemplified by groups such as methyl, ethyl, n-propyl, isopropyl, n-butyl, isobutyl, tert-butyl, n-hexyl, and the like.

“Alkenyl” refers to a straight or branched aliphatic group with at least one double bond. Typically, alkenyl groups have from 2 to 12 carbon atoms, from 2 to 8, from 2 to 6, or from 2 to 4 carbon atoms. Examples of alkenyl groups include ethenyl (—CH═CH₂), n-2-propenyl (allyl, —CH₂CH═CH₂), pentenyl, hexenyl, and the like.

“Alkynyl” refers to a straight or branched aliphatic group having at least 1 site of alkynyl unsaturation. Typically, alkynyl groups contain 2 to 12, 2 to 8, 2 to 6 or 2 to 4 carbon atoms. Examples of alkynyl groups include ethynyl (—C≡CH), propargyl (—CH₂C≡CH), pentynyl, hexynyl, and the like.

“Alkylene” refers to a bivalent saturated straight-chained hydrocarbon, e.g., C₁-C₆ alkylene includes —(CH₂)₆—, —CH₂—CH—(CH₂)₃CH₃, and the like. “Bivalent means that the alkylene group is attached to the remainder of the molecule through two different carbon atoms.

“Alkenylene” refers to an alkylene group with in which one carbon-carbon single bond is replaced with a double bond.

“Alkynylene” refers to an alkylene group with in which one carbon-carbon single bond is replaced with a triple bond.

“Aryl” used alone or as part of a larger moiety as in “aralkyl” refers to an aromatic carbocyclic group of from 6 to 14 carbon atoms having a single ring or multiple condensed rings. The term “aryl” also includes aromatic carbocycle(s) fused to cycloalkyl or heterocycloalkyl groups. Examples of aryl groups include phenyl, benzo[d][1,3]dioxole, naphthyl, phenantrenyl, and the like.

“Aryloxy” refers to an —OAr group, wherein O is an oxygen atom and Ar is an aryl group as defined above.

An “aralkyl group” is an alkyl group substituted with an aryl group. Examples of aralkyl groups include —CH₂-phenyl, wherein the phenyl group is optionally substituted on a substitutable ring carbon. The term “ring atom” is an atom such as C, N, O or S that is in the ring of an aromatic group, cycloalkyl group or non-aromatic heterocyclic ring.

A “substitutable ring atom” in an aromatic group is a ring carbon or nitrogen atom bonded to a hydrogen atom. The hydrogen can be optionally replaced with a suitable substituent group. Thus, the term “substitutable ring atom” does not include ring nitrogen or carbon atoms which are shared when two rings are fused. In addition, “substitutable ring atom” does not include ring carbon or nitrogen atoms when the structure depicts that they are already attached to a moiety other than hydrogen. Examples of suitable substituents on a substitutable ring carbon atom of an aryl group include optionally substituted C₁-C₆ alkyl. In a specific embodiment the aralkyl group is

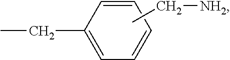

more specifically the alkyl amine is para to the methylene.

A “nitrogen-containing non-aromatic heterocyclic group” is a non-aromatic heterocyclic group with at least one nitrogen ring atom, and can be monocyclic, or polycyclic, for example, fused bicyclic or bridged bicyclic. Nitrogen-containing non-aromatic heterocyclic groups typically having three to fourteen members, preferably five to ten, in which one or more ring carbons, can each replaced by a heteroatom such as N, 0, or S, which can be saturated or unsaturated.

Examples of nitrogen-containing non-aromatic heterocyclic groups include pyrrolidinyl, piperazinyl, piperidinyl, morpholinyl

“Alkyl cycloalkyl” refers to an alkyl having at least one alkyl hydrogen atom replaced with a cycloalkyl moiety, such as —CH₂-cyclohexyl, —CH₂-cyclohexenyl, and the like.

“Heteroaryl” used alone or a part of a larger moiety as in “heteroaralkyl” refers to a 5 to 14 membered monocyclic, bicyclic or tricyclic heteroaromatic ring system, containing one to four ring heteroatoms independently selected from nitrogen, oxygen and sulfur. The term “heteroaryl” also includes heteroaromatic ring(s) fused to cycloalkyl or heterocycloalkyl groups. Particular examples of heteroaryl groups include optionally substituted pyridyl, pyrrolyl, pyrimidinyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadiazolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 1,2,3,4-tetrahydroquinolyl, 1,2,3,4-tetrahydroisoquinolyl, purinyl, pteridinyl, carbazolyl, xanthenyl, benzoquinolyl, and the like.

“Heteroaryloxy” refers to an —OHet group, wherein O is an oxygen atom and Het is a heteroaryl group as defined above.

“Heteroaralkyl” refers to an alkyl having at least one alkyl hydrogen atom replaced with a heteroaryl moiety, such as —CH₂-pyridinyl, —CH₂-pyrimidinyl, and the like.

“Alkoxy” refers to the group —O—R where R is “alkyl”, “cycloalkyl”, “alkenyl”, or “alkynyl”. Examples of alkoxy groups include for example, methoxy, ethoxy, ethenoxy, and the like.

“Alkyl heterocycloalkyl” refers to an alkyl having at least one alkyl hydrogen atom replaced with a heterocycloalkyl moiety, such as —CH₂-morpholino, —CH₂-piperidyl and the like.

“Alkoxycarbonyl” refers to the group —C(O)OR where R is “alkyl”, “alkenyl”, “alkynyl”, “cycloalkyl”, “heterocycloalkyl”, “aryl”, or “heteroaryl”.

“Hydroxyalkyl” and “alkoxyalkyl” are alky groups substituted with hydroxyl and alkoxy, respectively.

“Amino” means —NH₂; “alkylamine” and “dialkylamine” mean —NHR and —NR₂, respectively, wherein R is an alkyl group. “Cycloalkylamine” and “dicycloalkylamine” mean —NHR and —NR₂, respectively, wherein R is a cycloalkyl group. “Cycloalkylalkylamine” means —NHR wherein R is a cycloalkylalkyl group. “[Cycloalkylalkyl][alkyl]amine” means —N(R)₂ wherein one R is cycloalkylalkyl and the other R is alkyl.

Haloalkyl and halocycloalkyl include mono, poly, and perhaloalkyl groups where the halogens are independently selected from fluorine, chlorine, bromine and iodine.

Suitable substituents for “alkyl”, “alkenyl”, “alkynyl”, “cycloalkyl”, “heterocycloalkyl”, “aryl”, or “heteroaryl”, etc., are those which will form a stable compound of the invention. Examples of suitable substituents are those selected from the group consisting of halogen, —CN, —OH, —NH₂, (C₁-C₄)alkyl, (C₁-C₄)haloalkyl, aryl, heteroaryl, (C₃-C₇)cycloalkyl, (5-7 membered) heterocycloalkyl, —NH(C₁-C₆)alkyl, —N((C₁-C₆)alkyl)₂, (C₁-C₆)alkoxy, (C₁-C₆)alkoxycarbonyl, —CONH₂, —OCONH₂, —NHCONH₂, —N(C₁-C₆)alkylCONH₂, —N(C₁-C₆)alkylCONH(C₁-C₆)alkyl, —NHCONH(C₁-C₆)alkyl, —NHCON((C₁-C₆)alkyl)₂, —N(C₁-C₆)alkylCON((C₁-C₆)alkyl)₂, —NHC(S)NH₂, —N(C₁-C₆)alkylC(S)NH₂, —N(C₁-C₆)alkylC(S)NH(C₁-C₆)alkyl, —NHC(S)NH(C₁-C₆)alkyl, —NHC(S)N((C₁-C₆)alkyl)₂, —N(C₁-C₆)alkylC(S)N((C₁-C₆)alkyl)₂, —CONH(C₁-C₆)alkyl, —OCONH(C₁-C₆)alkyl —CON((C₁-C₆)alkyl)₂, —C(S)(C₁-C₆)alkyl, —S(O)ₚ(C₁-C₆)alkyl, —S(O)ₚNH₂, —S(O)ₚNH(C₁-C₆)alkyl, —S(O)ₚN((C₁-C₆)alkyl)₂, —CO(C₁-C₆)alkyl, —OCO(C₁-C₆)alkyl, —C(O)O(C₁-C₆)alkyl, —OC(O)O(C₁-C₆)alkyl, —C(O)H or —CO₂H. More particularly, the substituents are selected from halogen, —CN, —OH, —NH₂, (C₁-C₄)alkyl, (C₁-C₄)haloalkyl, (C₁-C₄)alkoxy, phenyl, and (C₃-C₇)cycloalkyl. Within the framework of this invention, said “substitution” is also meant to encompass situations where a hydrogen atom is replaced with a deuterium atom. p is an integer with a value of 1 or 2.

Pharmaceutically acceptable salts of the compounds disclosed herein are included in the present invention. For example, an acid salt of a compound containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

Salts of the compounds containing an acidic functional group can be prepared by reacting with a suitable base. Such a pharmaceutically acceptable salt can be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

### Pharmaceutical Compositions

The invention also provides pharmaceutical compositions comprising an effective amount of a compound Formula A, Formula B, or Formula I (e.g., including any of the formulae herein), or a pharmaceutically acceptable salt of said compound; and a pharmaceutically acceptable carrier. The carrier(s) are “pharmaceuticallyacceptable” in that they are not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), pulmonary, vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the patient therapeutics may be local, so as to be administered at the site of interest. Various techniques can be used for providing the patient compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. In one embodiment, the second therapeutic agent is one or more additional compounds of the invention.

In another embodiment, the second therapeutic agent may be selected from any compound or therapeutic agent known to have or that demonstrates advantageous properties when administered with a compound having the same mechanism of action as the APJ receptor compound of Formula A, Formula B, or Formula I.

In a particular embodiment, the second therapeutic is an agent useful in the treatment or prevention of a disease or condition selected from cardiovascular diseases, (e.g., hypertension and heart failure, such as congestive heart failure), cancer, diabetes, stem cell trafficking, fluid homeostasis, cell proliferation, immune function, obesity, metastatic disease, and HIV infection. In another embodiment, the second therapeutic is an agent useful in the treatment or prevention of a disease or condition selected from hypertension and heart failure, in particular, congestive heart failure.

For example, when the disease or condition is congestive heart failure, the second therapeutic agent can be selected from: ACE inhibitors, beta blockers, vasodilator, calcium channel blockers, loop diuretics, aldosterone antagonists, and angiotensin receptor blockers.

When the disease or condition being treated is hypertension, the second therapeutic agent can be selected from: α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

Centrally acting antiphypertensives include clonidine, guanabenz, guanfacine and methyldopa.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy. Preferably, the compound is present in the composition in an amount of from 0.1 to 50 wt. %, more preferably from 1 to 30 wt. %, most preferably from 5 to 20 wt. %.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily treatment dose or one of multiple daily treatment doses (e.g., about 1 to 4 or more times per day). When multiple daily treatment doses are used, the unit dosage form can be the same or different for each dose.

### Methods of Treatment

As used herein the term “subject” and “patient” typically means a human, but can also be an animal in need of treatment, e.g., companion animals (dogs, cats, and the like), farm animals (cows, pigs, horses, sheep, goats, and the like) and laboratory animals (rats, mice, guinea pigs, and the like).

The terms “treat” and “treating” are used interchangeably and include both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

As used herein, the term “effective amount” refers to an amount which, when administered in a proper dosing regimen, is sufficient to treat (therapeutically or prophylactically) the target disorder. For example, and effective amount is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The invention also includes methods of treating diseases, disorders or pathological conditions which benefit from modulation of the APJ receptor comprising administering an effective amount of an APJ receptor compound of the invention to a subject in need thereof. Diseases and conditions which can benefit from modulation (inhibition or activation) of the APJ receptor include, but are not limited to cardiovascular diseases, (e.g., hypertension and heart failure, such as congestive heart failure), cancer, diabetes, stem cell trafficking, fluid homeostasis, cell proliferation, immune function, obesity, metastatic disease, and HIV infection. In another embodiment, the second therapeutic is an agent useful in the treatment or prevention of a disease or condition selected from hypertension and heart failure, in particular, congestive heart failure and hypertrophic cardiomyopathy. In addition, the second therapeutic would be useful in the treatment or prevention of coronary artery disease, atherosclerosis, stable and unstable angina pectoris, restenosis, acute myocardial infarction, pulmonary hypertension, diseases related to cardiac ischemia, sudden heart death and for identifying therapeutics that modulate angiogenesis.

In one embodiment, APJ receptor compounds of the invention are useful as inotropic agents for use in patients with heart failure.

In another embodiment, the APJ receptor compounds of the invention can be administered for treatment of the hypertension.

In another embodiment, the APJ receptor compounds of the invention can be administered for treatment of HIV infection.

In an additional aspect, the APJ receptor compounds of the invention can be administered for treatment of tumor metastases.

In one embodiment, an effective amount of a compound of this invention can range from about 0.005 mg to about 5000 mg per treatment. In more specific embodiments, the range is from about 0.05 mg to about 1000 mg, or from about 0.5 mg to about 500 mg, or from about 5 mg to about 50 mg. Treatment can be administered one or more times per day (for example, once per day, twice per day, three times per day, four times per day, five times per day, etc.). When multiple treatments are used, the amount can be the same or different.

It is understood that a treatment can be administered every day, every other day, every 2 days, every 3 days, every 4 days, every 5 days, etc. For example, with every other day administration, a treatment dose can be initiated on Monday with a first subsequent treatment administered on Wednesday, a second subsequent treatment administered on Friday, etc. Treatment is typically administered from one to two times daily. Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

Alternatively, the effective amount of a compound of the invention is from about 0.01 mg/kg/day to about 1000 mg/kg/day, from about 0.1 mg/kg/day to about 100 mg/kg/day, from about 0.5 mg/kg/day to about 50 mg/kg/day, or from about 1 mg/kg/day to 10 mg/kg/day.

In another embodiment, any of the above methods of treatment comprises the further step of co-administering to said patient one or more second therapeutic agents. The choice of second therapeutic agent may be made from any second therapeutic agent known to be useful for co-administration with a compound that modulates the APJ receptor. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

### Kits

The present invention also provides kits for use to treat the target disease, disorder or condition. These kits comprise (a) a pharmaceutical composition comprising a compound of Formula A, Formula B, or Formula I, or a salt thereof, wherein said pharmaceutical composition is in a container; and (b) instructions describing a method of using the pharmaceutical composition to treat the target disease, disorder or condition.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

### General Methods for Preparing APJ Receptor Compounds

### Synthesis of Peptides

The peptide component (P) of the compounds of the invention can be synthesized by incorporating orthogonally protected amino acids in a step-wise fashion. Any suitable synthetic methods can be used. Traditional Fmoc or Boc chemistry can be easily adapted to provide the desired peptide component (P) of the compounds of the invention. Fmoc is generally preferred, because the cleavage of the Fmoc protecting group is milder than the acid deprotection required for Boc cleavage, which requires repetitive acidic deprotections that lead to alteration of sensitive residues, and increase acid catalyzed side reactions (Fields, G. B. et al. in Int. J Pept. Protein, 1990, 35, 161).

The peptides can be assembled linearly via Solid Phase Peptide Synthesis (SPPS), can be assembled in solution using modular condensations of protected or unprotected peptide components or a combination of both.

### Solid Phase Peptide Synthesis

For SPPS, an appropriate resin is chosen that will afford the desired moiety on the C-terminus upon cleavage. For example upon cleavage of the linear peptide, a Rink amide resin will provide a primary amide on the C-terminus, whereas a Rink acid resin will provide an acid. Rink acid resins are more labile than Rink amide resins and the protected peptide could also be cleaved and subsequently the free acid activated to react with amines or other nucleophiles. Alternatively, other resins could provide attachment of other moieties prior to acylation, leading to cleavage of an alkylated secondary amide, ester or other desired C-terminal modification. A review of commonly used resins and the functional moiety that results after cleavage can be found in manufacturer literature such as NovaBiochem or Advanced Chemtech catalogues.

Typically a resin is chosen such that after cleavage the C-terminus is an amide bond. Rink amide resin is a resin that results in a C-terminal amide during cleavage. The orthogonally protected Fmoc amino acids are added stepwise using methods well known in literature (Bodansky M., Principles of Peptide Synthesis (1993) 318p; Peptide Chemistry, a Practical Textbook (1993); Spinger-Verlag). These procedures could be done manually or by using automated peptide synthesizers.

The process involves activating the acid moiety of a protected amino acid, using activating agents such as HCTU, HBTU, HATU, PyBop or simple carbodiimides. Often an additive is used to decrease racemization during coupling such as HOBt or HOAt (Schnölzer, M. et al., Int. J. Pept. Protein Res., 1992, 40, 180). Manually, the coupling efficiency can be determined photometrically using a ninhydrin assay. If the coupling efficiency is below 98%, a second coupling may be desired. After the second coupling a capping step may be employed to prevent long deletion sequences to form, simplifying the purification of the desired final compound. With automation, second couplings are not commonly required, unless a residue is known to be problematic such as arginine.

Deprotection of the Fmoc is most commonly accomplished using piperidine (20%) in dimethylformamide (DMF). Alternatively other secondary amines may also be used such as morpholine, diethylamine or piperazine. This reaction is facile and normally is accomplished within 20 minutes using piperidine. After deprotection the resin is washed several times with DMF and DCM prior to coupling with the next residue. This process is repeated, assembling the peptide linearly until the sequence is complete. The final Fmoc is removed, which allows for coupling with the tether moiety.

In a preferred synthesis, the peptide is formed by SPPS accomplished manually or in an automated fashion using a commercially available synthesizer such as the CEM Microwave peptide synthesizer, Rainin Symphony synthesizer, or ABI 433 flow-through synthesizer or for parallel synthesis an Intavis MultiPep RS. Commercially available Rink Amide resin is used for synthesizing the C-terminal amide peptides (Rink, H., Tetrahedron Lett, 28, 4645, 1967). Although the NovaSyn TGR resin is preferred for the Intavis instrument and will also provide the C-terminal amide. Peptide synthesis reagents (coupling, deprotection agents) are commercially available and include HOBT, HBTU, HCTU (Novabiochem) as well as DMF, DCM, Piperidine, NMM, NMP, and DIEA (Sigma-Aldrich). Suitably protected amino acids for use in solid phase peptide synthesis are commercially available from many sources, including Sigma-Aldrich and CEM Corporation.

For example, a convenient preparation of peptides can be done in parallel at 5 μM scale using the INTAVIS ResPep RS with NovaSyn TGR resin with 0.25 mM loading using about 5 fold excess reagents, amino acids and coupling reagent (HCTU). Deprotection of Fmoc can be accomplished with 20% piperdine in DMF.

In another preferred synthesis, peptides can be synthesized using a microwave instrument using 10 eq of reagents. Deprotection of Fmoc can be accomplished with 20% piperidine in DMF followed by washing with DMF and DCM.

In both cases (i.e., Rink acid and Rink amide resins), final Fmoc deprotection of the N-terminus would leave a free amine after cleavage from the resin unless it is modified prior to cleavage. Compounds of the invention could be modified with tether moieties via couplings resulting in an amide, thioamide, sulfonamide, urea, thiourea, carbamate, thiocarbamate, carbamodithioate, imine, imidamide, or guanidine bonds. The amino terminus could be capped with a lipid such as palmitic acid.

### Amino Acid Reagents

The following commercially available orthogonally protected amino acids used can be used in the synthesis of compounds of the invention: Fmoc-Tyr(tBu)-OH, Fmoc-Ala-OH*H₂O, Fmoc-Arg(Pbf)-OH, Fmoc, Asn(Trt)-OH, Fmoc-Asp(tBu), Fmoc-Cys(tBu)-OH, Fmoc-Glu(tBu)-OH, Fmoc-Glx(Pbf)-OH, Fmoc-Gly-OH, Fmoc-His(Trt)-OH, Fmoc-Leu-OH, Fmoc-Ile-OH, Fmoc, Lys(tBu)-OH, Fmoc-Met-OH, Fmoc-Phe-OH, Fmoc-Ser(tBu)-OH, Fmoc-Thr(tBu)-OH, Fmoc-Typ-OH, and Fmoc-Val-OH. Additional amino acids suitable for incorporation into the compounds of the invention (e.g., D-amino acids, substituted amino acids and other protecting group variations) are also commercially available or synthesized by methods known in the art.

### Synthesis:

In the general operation of the Intavis ResPep SL (or MultiPep RS), 96 well plates having a small pore at the bottom of each well are used. Without resin in the well, the activated solutions would not have a very long residence time in the well plate. Using a resin with good swelling properties blocks the pore under normal gravity conditions allowing the activated amino acids to react with the resin for a prescribed time. Afterwards the instrument will activate a vacuum removing the solution from the resin and directing it to waste. Likewise the resin is washed and FMOC deprotection continues the cycle. The cycle repeats itself for each amino acid coupling until the elongation is complete. N-terminal lipidation is accomplished using the same protocol as an amino acid in the sequence. Final deprotection and cleavage is done by hand external to the instrument.

Preferred Protocol NovaSyn TGR resin is the preferred resin for use on the Intavis instrument. With its lower loading of 0.25 mmol/g (as compared to 0.6 for rink amide resin), it has better capability to swell up and hold the active chemicals during synthesis. The APJ compounds are synthesized in a 96-well format. To initiate synthesis, the resin has to be placed into each well of the 96 well plate. The recommended loading of each well is at a 5 μM scale. The total plate would require 0.48 mmol of resin which is ca. 1.92 g of resin (0.25/(96*0.005 mM)). About 10 mL of N-Methylpyroolidinone (NMP) is added to create a “slurry” of the resin which is transferred to the 96 wells with a multichannel pipettor, (100 uL to each well). The NMP is removed under vacuum filtration in the instrument.

The test sequences of compounds are uploaded to the Intavis using an excel spreadsheet (or alternatively could be entered by hand). The instrument program calculates the amounts of amino acids, coupling reagent (HCTU) needed with volumes and they are prepared using this printout. These quantities are loaded on the instrument at 0.5M dissolved in DMF using 5-10% excess. The program also calculates the base N-methylmorpholine (NMM) and piperidine (20% in DMF). If the sequences contain DG combinations 0.1M HOBT is added to the piperidine solution to minimize side reaction (Asp rearrangement).

A deprotection protocol begins the synthesis. The Fmoc deprotection step is set up to repeat three times (a triple Fmoc deprotection) using t 150 μL (10 min/cycle) throughout the entire synthesis. After the third cycles, the plate is rinsed with 1800 uL DMF, and the waste is extracted through the plate to the waste container. Likewise the amino acids are coupled three times (20 min/cycle) by adding 42.5 μL (1 eq) of activator, 13 μL (2 eq) base, 2 μL NMP (for solvent), and 44 μL (1.05 eq) of amino acid at 5 fold excess-to the loading of the resin in each well for each coupling. During the final cycle we Fmoc deprotect 4 times and the plate is washed again with DMF (1800 μL). Once the peptide sequence is complete, palmitic acid is coupled to the N-term of our sequences (4 cycles) using the same reagents and concentrations for each amino acid coupling. The palmitic acid is added from a solution of palmitic acid at 0.2M concentration due to solubility-.

### Cleavage:

After completion of the synthesis, the plates are washed with NMP. The plate is removed and placed into a collection apparatus external to the instrument. Added to each well is the deprotection cocktail: 400 μl TFA/70% MSA/TIS/DDT9:1:1:1. The plate is covered and kept at room temperature for 18-24 hr. The wells are evacuated under positive pressure (via a pipette) and the TFA solution is collected into a 96 well collection plate. The solution is transferred to a 2 mL HPLC vial using a disposable pipette. The vials are capped, labeled and loaded onto an agilent 1100 LC/MS equipped with mass based fraction collection using the Purification gradient below).

Fractions are collected in 96 well plates. Fractions containing the desired MW are combined and lyophilized. Purity analysis is performed on an Agilent 1100 HPLC using area under curve at 220 nm.

### Purification:

The aqueous HPLC mobile phase used for purification is 10 mM ammonium acetate buffer and acetonitrile is the organic modifier. To purify our crude compounds, an analytical Phenomenex column (10 micron C5 Luna column, 250 mm×4.6 mm) is used with a flow rate of 1.2 mL/min. The gradient runs 21 minutes including re-equilibration.

### Analytical Methods

The compounds of the invention are analyzed for purity by HPLC using the methods listed below. Purification is achieved by preparative HPLC.

Fast LC/MS Method
Column: Phenomenex Luna C-5 20×30 mm
Flow: 1.0 ml/min
Solvent A: 0.1% TFA in Type I water
Solvent B: 0.1% TFA in Acetonitrile
UV 220 nm
Injection: 20 ul
Gradient 5-95% B (7 minutes); 95-5% B (1 minute); 5% B (4 minutes)

Analytical Purity Method
Column: Phenomenex Luna C-5 20×30 mm
Flow: 1.0 ml/min
Solvent A: 0.1% TFA in Type I water
Solvent B: 0.1% TFA in Acetonitrile
UV: 220 nm
Injection: 20 ul
Gradient: 2-95% B (10 minutes); 95-2% B (2 minutes); 2% B (2 minutes)

Preparative LC/MS Method (CEM)
Column: Phenomenex Luna C-5 250 mmx 150 mm
Flow: 5 ml/min
Solvent A: 0.1% TFA in Type I water
Solvent B: 0.1% TFA in Acetonitrile
UV: 220 nm
Injection: 900 ul
Gradient: 35% B (5 minutes); 35-85% B (13 minutes); 85-35% B (0.5 minutes); 35% B (1.5 minutes)

Preparative LC/MS Method (Intavis)
Column: Phenomenex Luna C-5 250 mmx 4.6 mm
Flow: 1.2 ml/min
Solvent A: 10 mMAmmonium Acetate in Type I water
Solvent B: Acetonitrile
UV: 220 nm
Injection: 400 ul
Gradient: 35% B (5 minutes); 35-85% B (13 minutes); 85-35% B (0.5 minutes); 35% B (1.5 minutes)

### Synthesis of Selected APJ Compounds

The compounds listed below in Table 6 and pharmaceutically acceptable salts thereof were prepared according to the methods described herein. The lower case letters in the sequence represent the D-isomer of that amino acid. When the C-terminus is denoted as NH₂ this means that the —COOH group of the C-terminus in present as an amide (—CONH₂).

TABLE 4
APJ i1 Compounds

| Cmpd | | | | | Instru- |
|---|---|---|---|---|---|
| # | Tether | Linker | Sequence | terminus | ment |
| 1 | Pal | —C(O)— | TVFRSSEkRRSADIFI | NH₂ | Intavis |
| | | | (SEQ ID NO: 2) | | |
| 20 | Pal | —C(O)— | QVFRSSEkRRSADIFI | NH₂ | Intavis |
| | | | (SEQ ID NO: 21) | | |
| 24 | Pal | —C(O)— | TVFRSQEkRRSADIFI | NH₂ | Intavis |
| | | | (SEQ ID NO: 25) | | |
| 68 | Pal | —C(O)— | GVFRSSEkRRSADIFI | NH₂ | Intavis |
| | | | (SEQ ID NO: 69) | | |
| 70 | Pal | —C(O)— | TVGRSSEkRRSADIFI | NH₂ | Intavis |
| | | | (SEQ ID NO 71) | | |

(SEQ ID NO: 2)
Compound 1 (Pal-TVFRSSEkRRSADIFI-amide)

Compound 1 was synthesized as described above using the Intavis MultiPep RS using Rink amide resin at 5 μmol scale. Amino acids were coupled sequentially as described above following the sequence from C-term to N-term.

Following deprotection of the Fmoc group on the N-terminal residue serine, the N-terminal amine was capped with Palmitic acid (2.5 eq×2 cycles.). The compound was manually cleaved from the resin by exposure to cleavage cocktail containing TFA MSA, TIS, DDT, and water (82: 4.5:4.5:4.5:4.5; 400 uL) for 12 hours. The cocktail was filtered through the 96-well plate into a collection plate and each well was transferred to a 2 mL RPHPLC vial for direct injection onto HPLC/MS system utilizing mass based fraction collection. Fractions with correct MW were pooled and lyophilized and finally analyzed for purity using Method A to yield 1.5 mg of Compound 1 (Purity 83%).

(SEQ ID NO: 21)
Compound 20-(Pal-QVFRSSEkRRSADIFI-amide)

Compound 20 was synthesized as described above using the Intavis MultiPep RS using Rink amide resin at 5 μmol scale. Amino acids were coupled sequentially as described above following the sequence from C-term to N-term.

Following deprotection of the Fmoc group on the N-terminal residue Serine. the N-terminal amine was capped with Palmitic acid (2.5 eq×4 cycles). The compound was manually cleaved from the resin by exposure to cleavage cocktail containing TFA MSA, TIS, DDT, and water (82: 4.5:4.5:4.5:4.5; 400 uL) for 12 hours. The cocktail was filtered through the 96-well plate into a collection plate and each well was transferred to a 2 mL RPHPLC vial for direct injection onto HPLC/MS system utilizing mass based fraction collection Fractions with correct MW were pooled and lyophilized and finally analyzed for purity using Method A to yield 2.3 mg of Compound 20 (Purity 72.4%).

( SEQ ID NO: 25)
Compound 24-(Pal-TVFRSQEkRRSADIFI-amide)

Compound 24 was synthesized as described above using the Intavis MultiPep RS using Rink amide resin at 5 μmol scale. Amino acids were coupled sequentially as described above following the sequence from C-term to N-term.

Following deprotection of the Fmoc group on the N-terminal residue Serine. the N-terminal amine was capped with Palmitic acid (2.5 eq×4 cycles). The compound was manually cleaved from the resin by exposure to cleavage cocktail containing TFA MSA, TIS, DDT, and water (82: 4.5:4.5:4.5:4.5; 400 uL) for 12 hours. The cocktail was filtered through the 96-well plate into a collection plate and each well was transferred to a 2 mL RPHPLC vial for direct injection onto HPLC/MS system utilizing mass based fraction collection Fractions with correct MW were pooled and lyophilized and finally analyzed for purity using Method A to yield 4.3 mg of Compound 24 (purity 73.7%).

( SEQ ID NO: 69)
Compound 68-(Pal-GVFRSSEkRRSADIFI-amide)

Compound 68 was synthesized as described above using the Intavis MultiPep RS using Rink amide resin at 5 μmol scale. Amino acids were coupled sequentially as described above following the sequence from C-term to N-term.

Following deprotection of the Fmoc group on the N-terminal residue Serine. the N-terminal amine was capped with Palmitic acid (2.5 eq×4 cycles). The compound was manually cleaved from the resin by exposure to cleavage cocktail containing TFA MSA, TIS, DDT, and water (82: 4.5:4.5:4.5:4.5; 400 uL) for 12 hours. The cocktail was filtered through the 96-well plate into a collection plate and each well was transferred to a 2 mL RPHPLC vial for direct injection onto HPLC/MS system utilizing mass based fraction collection Fractions with correct MW were pooled and lyophilized and finally analyzed for purity using Method A to yield 2 mg of Compound 68, Purity (77.2%).

( SEQ ID NO: 71)
Compound No. 70-(Pal-TVGRSSEkRRSADIFI-amide)

Compound 70 was synthesized as described above using the Intavis MultiPep RS using Rink amide resin at 5 μmol scale Amino acids were coupled sequentially as described above following the sequence from C-term to N-term.

Following deprotection of the Fmoc group on the N-terminal residue Serine. the N-terminal amine was capped with Palmitic acid (2.5 eq×4 cycles). The compound was manually cleaved from the resin by exposure to cleavage cocktail containing TFA MSA, TIS, DDT, and water (82: 4.5:4.5:4.5:4.5; 400 uL) for 12 hours. The cocktail was filtered through the 96-well plate into a collection plate and each well was transferred to a 2 mL RPHPLC vial for direct injection onto HPLC/MS system utilizing mass based fraction collection Fractions with correct MW were pooled and lyophilized and finally analyzed for purity using Method A to yield 3.3 mg of Compound 70, Purity (85.8%).

In Table 1, specific compounds of the invention are described. For each of the compounds in Table 1, the N-terminal amino acid residue is bonded to —C(O)— (the Linker “L” of Formula A, Formula B, or Formula I) and the —C(O)— is in turn bonded to the Tether (T). In the case of the compounds in Table 1, the Tether (T) is CH₃(CH₂)₁₄ derived from palmitic acid:

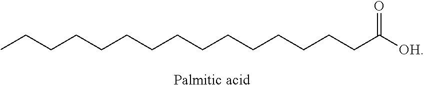 [SMILES: CCCCCCCCCCCCCCCC(=O)O]

For example, the second compound listed in Table 2 shows the sequence as Pal-QVFRSSEkRRSADIFI-NH2 (SEQ ID NO 21), the Tether as Pal, the linker as —C(O)— and the C-terminal carbon as an amide. This means that this compound has the following structure when drawn out long hand, with the understanding that the flanked sequence of amino acids is in the form of a typical peptide:

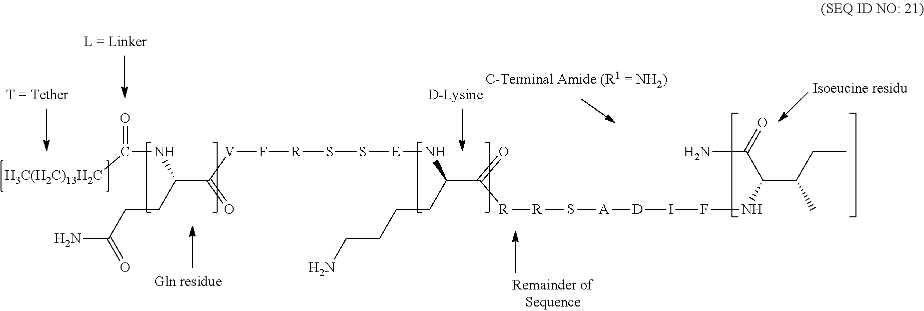

Additional compounds that were synthesized following the above-described method are listed in Table 5.

TABLE 5

| Compound | | SEQ | | | | Obs |
|---|---|---|---|---|---|---|
| # | Sequences | ID NO: | Tether | Linker | MW | Mass |
| 1 | TVFRSSEkRRSADIFI | 2 | Pal | —C(O)— | 2149.58 | 1075 |
| 2 | TFFRSSEkRRSADIFI | 3 | Pal | —C(O)— | 2197.62 | 1099.3 |
| 3 | TVFRSSEkFRSADIFI | 4 | Pal | —C(O)— | 2140.57 | 714.3 |
| 4 | TVFRSSEkRRSAFIFI | 5 | Pal | —C(O)— | 2181.66 | 728 |
| 5 | TVFRSSEkRRSADFFI | 6 | Pal | —C(O)— | 2183.59 | 728.5 |
| 6 | TVFRSSEkRRSADIFF | 7 | Pal | —C(O)— | 2183.59 | 1092.7 |
| 7 | HVFRSSEkRRSADIFI | 8 | Pal | —C(O)— | 2185.61 | 1093.7 |
| 8 | THFRSSEkRRSADIFI | 9 | Pal | —C(O)— | 2187.59 | 1094.4 |
| 9 | TVHRSSEkRRSADIFI | 10 | Pal | —C(O)— | 2139.54 | 1070 |
| 10 | TVFHSSEkRRSADIFI | 11 | Pal | —C(O)— | 2130.53 | 1066.6 |
| 11 | TVFRSHEkRRSADIFI | 12 | Pal | —C(O)— | 2199.64 | 1100.7 |
| 12 | TVFRSSHkRRSADIFI | 13 | Pal | —C(O)— | 2157.6 | 1079.7 |
| 13 | TVFRSSEkHRSADIFI | 14 | Pal | —C(O)— | 2130.53 | 711 |
| 14 | TVFRSSEkRHSADIFI | 15 | Pal | —C(O)— | 2130.53 | 1065.8 |
| 15 | TVFRSSEkRRSHDIFI | 16 | Pal | —C(O)— | 2215.64 | 739.4 |
| 16 | TVFRSSEkRRSAHIFI | 17 | Pal | —C(O)— | 2171.63 | 1086.5 |
| 17 | TVFRSSEkRRSADHFI | 18 | Pal | —C(O)— | 2173.56 | 725.3 |
| 18 | TVFRSSEkRRSADIHI | 19 | Pal | —C(O)— | 2139.54 | 714 |
| 19 | TVFRSSEkRRSADIFH | 20 | Pal | —C(O)— | 2173.56 | 725.3 |
| 20 | QVFRSSEkRRSADIFI | 21 | Pal | —C(O)— | 2176.6 | 726.4 |
| 21 | TQFRSSEkRRSADIFI | 22 | Pal | —C(O)— | 2178.58 | 727 |
| 22 | TVFQSSEkRRSADIFI | 23 | Pal | —C(O)— | 2121.52 | 708 |
| 23 | TVFRQSEkRRSADIFI | 24 | Pal | —C(O)— | 2190.63 | 1095.8 |
| 24 | TVFRSQEkRRSADIFI | 25 | Pal | —C(O)— | 2190.63 | 1095.8 |
| 25 | TVFRSSQ1aRSADIFI | 26 | Pal | —C(O)— | 2148.59 | 1075.2 |
| 26 | TVFRSSEkQRSADIFI | 27 | Pal | —C(O)— | 2121.52 | 1061.6 |
| 27 | TVFRSSEkRQSADIFI | 28 | Pal | —C(O)— | 2121.52 | 1061.8 |
| 28 | TVFRSSEkRRSQDIFI | 29 | Pal | —C(O)— | 2206.63 | 1104.2 |
| 29 | TVFRSSEkRRSAQIFI | 30 | Pal | —C(O)— | 2162.62 | 1082.3 |
| 30 | TVFRSSEkRRSADQFI | 31 | Pal | —C(O)— | 2164.55 | 722.3 |
| 31 | TVFRSSEkRRSADIQI | 32 | Pal | —C(O)— | 2130.53 | 1066.2 |
| 32 | TVFRSSEkRRSADIFQ | 33 | Pal | —C(O)— | 2164.55 | 1082.8 |
| 33 | DVFRSSEkRRSADIFI | 34 | Pal | —C(O)— | 2163.56 | 722 |
| 34 | TDFRSSEkRRSADIFI | 35 | Pal | —C(O)— | 2165.53 | 722.7 |
| 35 | TVDRSSEkRRSADIFI | 36 | Pal | —C(O)— | 2117.49 | 706.7 |
| 36 | TVFRDSEkRRSADIFI | 37 | Pal | —C(O)— | 2177.59 | 1089.7 |
| 37 | TVFRSDEkRRSADIFI | 38 | Pal | —C(O)— | 2177.59 | 726.7 |
| 38 | TVFRSSEkRRDADIFI | 39 | Pal | —C(O)— | 2177.59 | 726.7 |
| 39 | TVFRSSEkRRSDDIFI | 40 | Pal | —C(O)— | 2193.59 | 732 |
| 40 | TVFRSSEkRRSADDFI | 41 | Pal | —C(O)— | 2151.51 | 1076.3 |
| 41 | TVFRSSEkRRSADIFD | 42 | Pal | —C(O)— | 2151.51 | 1076.8 |
| 42 | PVFRSSEkRRSADIFI | 43 | Pal | —C(O)— | 2145.59 | 1073.7 |
| 43 | TPFRSSEkRRSADIFI | 44 | Pal | —C(O)— | 2147.56 | 1074.7 |
| 44 | TVFPSSEkRRSADIFI | 45 | Pal | —C(O)— | 2090.51 | 1046.1 |
| 45 | TVFRPSEkRRSADIFI | 46 | Pal | —C(O)— | 2159.62 | 1080 |
| 46 | TVFRSPEkRRSADIFI | 47 | Pal | —C(O)— | 2159.62 | 720.7 |
| 47 | TVFRSSPIcRRSADIFI | 48 | Pal | —C(O)— | 2117.58 | 1059.3 |
| 48 | TVFRSSEPRRSADIFI | 49 | Pal | —C(O)— | 2118.52 | 1059.7 |
| 49 | TVFRSSEPRRSADIFI | 50 | Pal | —C(O)— | 2090.51 | 1045.8 |
| 50 | TVFRSSEkRPSADIFI | 51 | Pal | —C(O)— | 2090.51 | 697.7 |
| 51 | TVFRSSEkRRPADIFI | 52 | Pal | —C(O)— | 2159.62 | 1080.8 |
| 52 | TVFRSSEkRRSPDIFI | 53 | Pal | —C(O)— | 2175.61 | 1088.3 |
| 53 | TVFRSSEkRRSAPIFI | 54 | Pal | —C(O)— | 2131.61 | 1066.4 |
| 54 | TVFRSSEkRRSADPFI | 55 | Pal | —C(O)— | 2133.54 | 1067.6 |
| 55 | TVFRSSEkRRSADIPI | 56 | Pal | —C(O)— | 2099.52 | 700.7 |
| 56 | TVFRSSEkRRSADIFP | 57 | Pal | —C(O)— | 2133.54 | 712 |
| 57 | T(Aib)FRSSEkRRSADIFI | 58 | Pal | —C(O)— | 2135.55 | 1068.2 |
| 58 | TVF(Aib)SSEkRRSADIFI | 59 | Pal | —C(O)— | 2078.5 | 693 |
| 59 | TVFRS(Aib)EkRRSADIFI | 60 | Pal | —C(O)— | 2147.6 | 716.7 |
| 60 | TVFRSSE(Aib)RRSADIFI | 61 | Pal | —C(O)— | 2106.51 | 702.9 |
| 61 | TVFRSSEk(Aib)RSADIFI | 62 | Pal | —C(O)— | 2078.5 | 693.5 |
| 62 | TVFRSSEkR(Aib)SADIFI | 63 | Pal | —C(O)— | 2078.5 | 693.7 |
| 63 | TVFRSSEkRR(Aib)ADIFI | 64 | Pal | —C(O)— | 2147.6 | 716.6 |
| 64 | TVFRSSEkRRS(Aib)DIFI | 65 | Pal | —C(O)— | 2163.6 | 722 |
| 65 | TVFRSSEkRRSA(Aib)IFI | 66 | Pal | —C(O)— | 2119.59 | 707.4 |
| 66 | TVFRSSEkRRSADI(Aib)I | 67 | Pal | —C(O)— | 2087.51 | 696.7 |
| 67 | TVFRSSEkRRSADIF(Aib) | 68 | Pal | —C(O)— | 2121.52 | 708 |
| 68 | GVFRSSEkRRSADIFI | 69 | Pal | —C(O)— | 2105.53 | 702.7 |
| 69 | TGFRSSEkRRSADIFI | 70 | Pal | —C(O)— | 2107.5 | 703.3 |
| 70 | TVGRSSEkRRSADIFI | 71 | Pal | —C(O)— | 2059.46 | 687.3 |
| 71 | TVFGSSEkRRSADIFT | 72 | Pal | —C(O)— | 2050.44 | 684.2 |
| 72 | TVFRGSEkRRSADIFI | 73 | Pal | —C(O)— | 2119.55 | 707.6 |
| 73 | TVFRSGEkRRSADIFI | 74 | Pal | —C(O)— | 2119.55 | 707.3 |
| 74 | TVFRSSGkRRSADIFI | 75 | Pal | —C(O)— | 2077.52 | 693.3 |
| 75 | TVFRSSEkGRSADIFI | 76 | Pal | —C(O)— | 2050.44 | 684.3 |
| 76 | TVFRSSEkRGSADIFI | 77 | Pal | —C(O)— | 2050.44 | 684.3 |
| 77 | TVFRSSEkRRGADIFI | 78 | Pal | —C(O)— | 2119.55 | 707.3 |
| 78 | TVFRSSEkRRSAGIFI | 79 | Pal | —C(O)— | 2091.54 | 698 |
| 79 | TVFRSSEkRRSADIGI | 80 | Pal | —C(O)— | 2059.46 | 687.3 |
| 80 | TVFRSSEkRRSADIFG | 81 | Pal | —C(O)— | 2093.47 | 698.7 |
| 81 | TVDRSSEyRRSADIFI | 82 | Pal | —C(O)— | 2152.49 | 718 |
| 82 | TVDRSSEKRRSADIFI | 83 | Pal | —C(O)— | 2237.64 | 746.7 |
| 83 | TVFRSSREKRRSADIFI- | 1 | Pal | —C(O)— | 2374.87 | 1188 |
| | piperazine (N-attached) | | | | | |
| 84 | TVFRSSREKRRSADIFI-NHR₄¹ | 1 | Pal | —C(O)— | 2424.93 | |
| | R₄¹ = xylylenediamine | | | | | |
| 85 | TVFRSSREKRRSADIFI-NHR₄¹ | 1 | Pal | —C(O)— | 2906.46 | 969 |
| | R₄¹ = [CH₂CH₂O]₁₂CH₂CH₂CO₂H | | | | | |
| 86 | TVFRSSREKRRSADIFI-NHR₄¹ | 1 | Pal | —C(O)— | 2905.47 | |
| | R₄¹ = [CH₂CH₂O]₁₂CH₂CH₂CONH₂ | | | | | |
| 87 | TVFQSEkRRSADQFI | 84 | Pal | —C(O)— | 2049.42 | 683.9 |

### Methods of Screening

### Functional Assays

Functional assays suitable for use in detecting and characterizing GPCR signaling include Gene Reporter Assays and Calcium Flux assays, cAMP and kinase activation assays. Several suitable assays are described in detail below.

### Gene Reporter Assays

Cells expressing the APJ receptor can be transiently or stably transfected with a reporter gene plasmid construct containing an enhancer element which responds to activation of a second messenger signaling pathway or pathways, thereby controlling transcription of a cDNA encoding a detectable reporter protein. APJ expression can be the result of endogenous expression on a cell line or cell type or the result of stable or transient transfection of DNA encoding the receptor of interest into a cell line by means commonly used in the art. Immortalized cell lines or primary cell cultures can be used.

If the activated pathway is stimulatory (e.g., Gs or Gq), agonist activity results in activation of transcription factors, in turn causing an increase in reporter gene transcription, detectable by an increase in reporter activity. To test for agonist or inverse agonist activity, cells expressing the APJ receptor and the reporter gene construct can be challenged by the test compound for a predetermined period of time (e.g., 2-12 hours, typically 4 hours). Cells can then be assessed for levels of reporter gene product. Inverse agonists will suppress levels of reporter to below basal levels in a dose dependent manner To test for antagonist or inhibitory activity through a stimulatory pathway, cells expressing both the APJ receptor and the reporter gene construct can be activated by a receptor agonist to increase gene reporter product levels. Treatment with antagonists will counter the effect of agonist stimulation in a dose- and receptor-dependent manner.

To test for agonist activity on receptor signaling through an inhibitory pathway (eg, Gi, which couples to APJ), cells can be treated with a systematic activator (e.g., forskolin) to increase levels of reporter gene product. Activation of Gi by treatment with receptor agonist will inhibit this expression by inhibiting adenylyl cyclase. To screen for antagonist activity, test compounds can be assessed for the ability to counter agonist inhibition of adenylyl cyclase, resulting in increase reporter transcription.

Alternatively, a plasmid construct expressing the promiscuous G-protein Ga16 can be used to obtain a positive signal from a GPCR which normally couples to an inhibitory G-protein. Co-expression of the chimeric G-protein Gaq/Gai5 (Coward et al. Analytical Biochemistry 270, 242-248 (1999)) allows coupling to Gi-coupled receptors and conversion of second messenger signaling from the inhibitory Gi pathway to the stimulatory Gq pathway. Agonist and antagonist assessment in these systems is the same as the stimulatory pathways. Well-to-well variation caused by such factors as transfection efficiency, unequal plating of cells, and cell survival rates can be normalized in transient transfection assays by co-transfecting a constitutively expressing reporter gene with a non-interfering signal independent of the regulated reporter.

### Calcium Flux Assay

Calcium Flux Assay is one of the most popular cell-based GPCR functional assays. It most often uses calcium sensing fluorescent dyes such as fura2 AM, fluo-4 and Calcium-4 to measure changes in intracellular calcium concentration. It is used mainly to detect GPCR signaling via Gαq subunit. Activation of these Gq-coupled GPCRs leads to activation of phospholipase C, which subsequently leads to increase in inositol phosphate production. IP3 receptors on endoplasmic reticulum sense the change then release calcium into cytoplasm. Intracellular calcium binding to the fluorescent dyes can be detected by instruments that quantify fluorescent intensities, such as FLIPR Tetra, Flexstation (MDS) and FDSS (Hamamatsu). In additional to assess Gq-couple receptor signaling, calcium flux assay can also be used to study Gs and Gi couple receptors by co-expressing CNG (cyclic nucleotide gated calcium channel) or chimeric G-proteins (Gqi5, Gsi5 for example). Activation of some Gi-coupled receptors can also be detected by calcium flux assay via Gβγ mediated phospholipase C activation.

### APJ Testing

An example of the use of the calcium flux assay can be assessing Apelin activation of APJ receptors in Molt3 human cell lines or in Rat RBL cells stably transfected with APJ. Cells can be seeded into 96-well black plates with clear bottom at 200K/well in Hank's balanced salt solution with 20 mM HEPES, 0.1% BSA. After dye loaded by incubating in Calcium-4 dye at room temperature for 1 hour, cell plates can be placed in Flexstation 3. The addition of test compound or reference antagonists can be done either by manual pipetting or by liquid handling on Flexstation. The latter allows the assessment of agonist activity of the test compound. After incubation of 15 minutes at 37° C. Apelin can be added on Flexstation and receptor activation can be assessed by measuring changes in fluorescent intensity. This mode of assay also allows the detection of agonists and agonistic modulators of APJ activity.

HTRF cAMP Assay and IP-One Assay (Cisbio)

HTRF (homogeneous time resolved fluorescence) is a technology developed by Cisbio Bioassays based on TR-FRET (time-resolved fluorescence resonance energy transfer). Cisbio Bioassays has developed a wide selection of HTRF-based assays compatible with whole cells, thereby enabling functional assays to run under more physiological conditions. The IP-One assays are competitive immunoassays using cryptate-labeled anti-IP1 monoclonal antibody and d2-labeled IP1. IP1 is a relatively stable downstream metabolite of IP3, and accumulates in cells following Gq receptor activation.

cAMP kits based on a competitive immunoassay using cryptate-labeled anti-cAMP antibody and d2-labeled cAMP were used to assay the effects of APJ compounds of the present invention. This assay measures the increase in intracellular cAMP upon Gs-coupled receptor activation as well as decrease in forskolin (or a more soluble version of forskolin—NKH477) stimulated increase in cAMP upon Gi-coupled receptor activation. For example, treatment of HEK cells stably expressing the Gi-coupled receptor APJ with its endogenous ligand Apelin inhibited NKH477 stimulated increase in cAMP with an EC₅₀ of 5 e-10 M. A second example of the use of the cAMP assay can be assessing Apelin activation of APJ receptor in Human TRex cells, which express APJ upon induction of doxycyline. Cells are treated with lnG/mL doxycycline and seeded in all white 96 well plates @ 40 k/well overnight. When Human TRex cells express APJ, Apelin inhibited NKH477 stimulated increase in cAMP with an EC₅₀ of 2 e-10 M. When Human TRex cells are not induced and therefore do not express the APJ receptor, Apelin does not inhibit NKH477 stimulated cAMP. This cell lines allows the assessment of activity of the compounds with and without the APJ receptor.

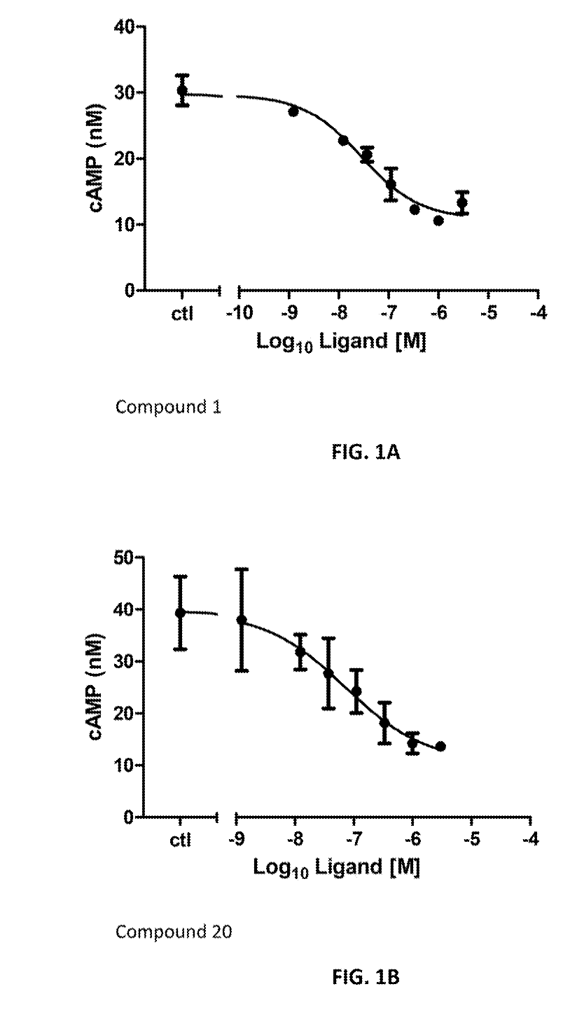

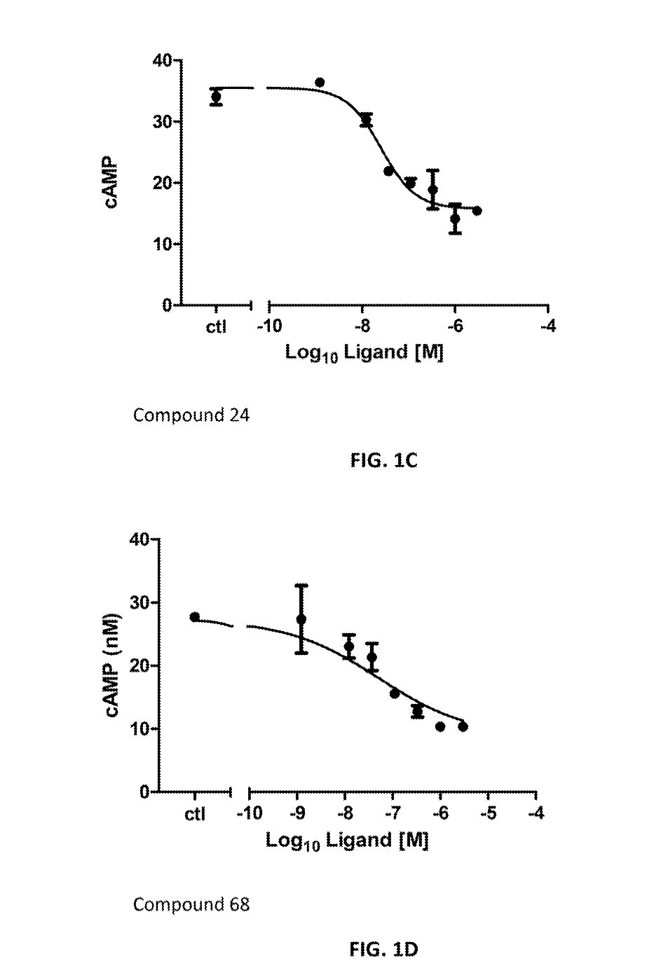

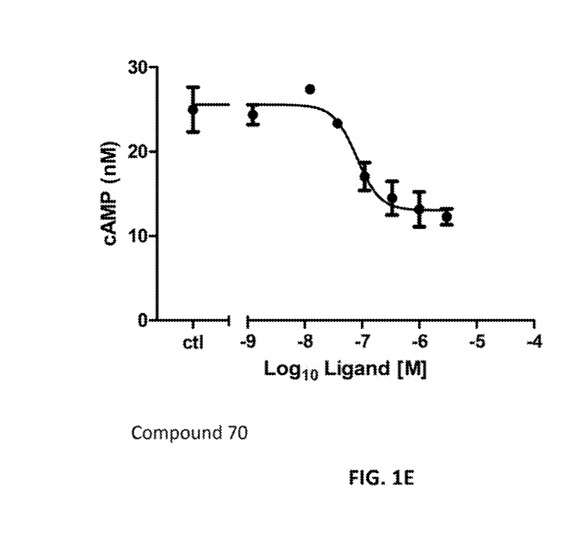

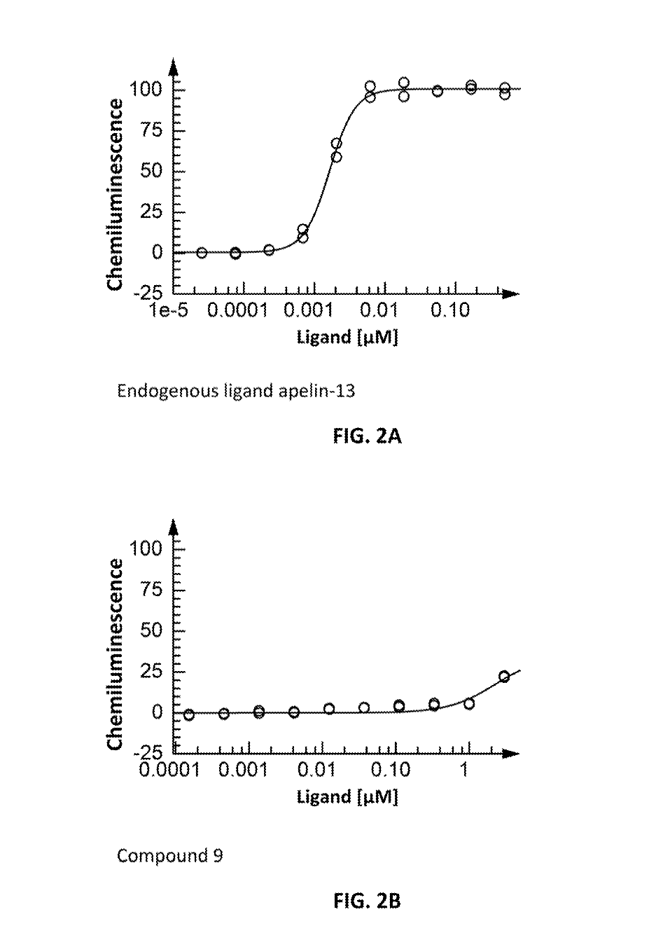

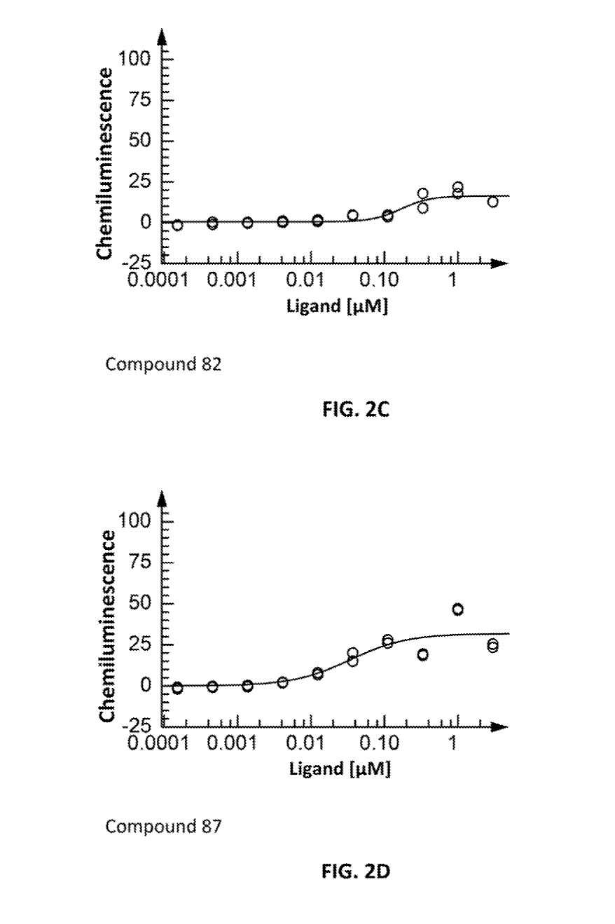

Representative data for this assay are described for Compounds 1, 20, 24, 68 and in FIGS. 1A, 1B, 1C, 1D and 1E respectively. Further testing of compounds was conducted and the results are set forth in Table 6. For the data in Table 6

*****=EC₅₀<100 nM and % histamine<35%
****=EC₅₀ 101-500 nM and % histamine<35%
***=EC₅₀<100 nM and % histamine>35%
**=EC₅₀ 101-500 nM and % histamine>35%
*=EC₅₀>500 nM and % histamine>35%

TABLE 6

| | | | APJ-hTREX | Histimine | |
|---|---|---|---|---|---|
| Compound | | % IA | EC50 | (% vs. | |
| # | Sequences | (@ 300 nM) | (nM) | 48/40) | Ranking |
| 1 | TVFRSSEkRRSADIFI | 81 | 56.75 | 33 | ***** |
| | (SEQ ID NO: 2) | | | | |
| 2 | TFFRSSEkRRSADIFI | 59 | — | 53 | |
| | (SEQ ID NO: 3) | | | | |
| 3 | TVFRSSEkFRSADIFI | 71 | 242.55 | 29 | **** |
| | (SEQ ID NO: 4) | | | | |
| 4 | TVFRSSEkRRSAFIFI | 65 | — | 118 | |
| | (SEQ ID NO: 5) | | | | |
| 5 | TVFRRSEkRRSADFFI | 81 | — | 89 | |
| | (SEQ ID NO: 6) | | | | |
| 6 | TVFRSSEkRRSADIFF | 76 | 49.2 | 70 | *** |
| | (SEQ ID NO: 7) | | | | |
| 7 | HVF4SSEkRRSADIFI | 50 | — | 60 | |
| | (SEQ ID NO: 8) | | | | |
| 8 | THFRSSEkRRSADIFI | 63 | — | 58 | |
| | (SEQ ID NO: 9) | | | | |
| 9 | TVHRSSEkRRSADIFI | 75 | 79.55 | 36 | *** |
| | (SEQ ID NO: 10) | | | | |
| 10 | TVFHSSEkRRSADIFI | 55 | — | 10 | |
| | (SEQ ID NO: 11) | | | | |
| 11 | TVFRSHEkRRSADIFI | 68 | 65.05 | 38 | *** |
| | (SEQ ID NO: 12) | | | | |
| 12 | TVFRSSHkRRSADIFI | 73 | — | 62 | |
| | (SEQ ID NO: 13) | | | | |
| 13 | TVFRSSEkHRSADIFI | 56 | — | 29 | |
| | (SEQ ID NO: 14) | | | | |
| 14 | TVFRSSEkRHSADIFI | 74 | 253 | 20 | **** |
| | (SEQ ID NO: 15) | | | | |
| 15 | TVFRSSEkRRSHDIFI | 77 | — | 103 | |
| | (SEQ ID NO: 16) | | | | |
| 16 | TVFRSSEkRRSAHIFI | 63 | — | 107 | |
| | (SEQ ID NO: 17) | | | | |
| 17 | TVFRSSEkRRSADHFI | 83 | — | 113 | |
| | (SEQ ID NO: 18) | | | | |
| 18 | TVFRSSEkRRSADIHI | 56 | — | 62 | |
| | (SEQ ID NO: 19) | | | | |
| 19 | TVFRSSEkRRSADIFH | 53 | — | 104 | |
| | (SEQ ID NO: 20) | | | | |
| 20 | QVFRSSEkRRSADIFI | 61 | 60.65 | 29 | ***** |
| | (SEQ ID NO: 21) | | | | |
| 21 | TQFRSSEkRRSADIFI | 72 | 40.95 | 39 | *** |
| | (SEQ ID NO: 22) | | | | |
| 22 | TVFQSSEkRRSADIFI | 59 | — | 2 | |
| | (SEQ ID NO: 23) | | | | |
| 23 | TVFRQSEkRRSADIFI | 55 | — | 26 | |
| | (SEQ ID NO: 24) | | | | |
| 24 | TVFRSQEkRRSADIFI | 70 | 42.6 | 22 | ***** |
| | (SEQ ID NO: 25) | | | | |
| 25 | TVFRSSQkRRSADIFI | 45 | — | 49 | |
| | (SEQ ID NO: 26) | | | | |
| 26 | TVFRSSEkQRSADIFI | 6 | 112 | −1 | **** |
| | (SEQ ID NO: 27) | | | | |
| 27 | TVFRSSEkRQSADIFI | 7 | — | 4 | |
| | (SEQ ID NO: 28) | | | | |
| 28 | TVFRSSEkRRSQDIFI | 8 | — | 16 | |
| | (SEQ ID NO: 29) | | | | |
| 29 | TVFRSSEkRRSAQIFI | 9 | — | 92 | |
| | (SEQ ID NO: 30) | | | | |
| 30 | TVFRSSEkRRSADQFI | 98 | 84.6 | 63 | *** |
| | (SEQ ID NO: 31) | | | | |
| 31 | TVFRSSEkRRSADIQI | 67 | — | 68 | |
| | (SEQ ID NO: 32) | | | | |
| 32 | TVFRSSEkRRSADIFQ | 55 | — | 69 | |
| | (SEQ ID NO: 33) | | | | |
| 33 | DVFRSSEkRRSADIFI | 61 | 106.8 | 11 | **** |
| | (SEQ ID NO: 34) | | | | |
| 34 | TDFRSSEkRRSADIFI | 44 | — | 17 | |
| | (SEQ ID NO: 35) | | | | |
| 35 | TVDRSSEkRRSADIFI | 70 | 141.975 | 4 | **** |
| | (SEQ ID NO: 36) | | | | |
| 36 | TVFRDSEkRRSADIFI | 49 | — | 6 | |
| | (SEQ ID NO: 37) | | | | |
| 37 | TVFRSDEkRRSADIFI | 50 | — | 8 | |
| | (SEQ ID NO: 38) | | | | |
| 38 | TVFRSSEkRRDADIFI | 46 | 732 | 13 | * |
| | (SEQ ID NO: 39) | | | | |
| 39 | TVFRSSEkRRSDDIFI | 27 | — | 8 | |
| | (SEQ ID NO: 40) | | | | |
| 40 | TVFRSSEkRRSADDFI | 71 | 48.2 | 46 | *** |
| | (SEQ ID NO: 41) | | | | |
| 41 | TVFRSSEkRRSADIFD | 60 | — | 48 | |
| | (SEQ ID NO: 42) | | | | |
| 42 | PVFRSSEkRRSADIFI | 83 | — | 36 | |
| | (SEQ ID NO: 43) | | | | |
| 43 | TPFRSSEkRRSADIFI | 39 | — | 30 | |
| | (SEQ ID NO: 44) | | | | |
| 44 | TVFPSSEkRRSADIFI | 74 | 110.65 | 7 | **** |
| | (SEQ ID NO: 45) | | | | |
| 45 | TVFRPSEkRRSADIFI | 70 | — | 37 | |
| | (SEQ ID NO: 46) | | | | |
| 46 | TVFRSPEkRRSADIFI | 77 | — | 50 | |
| | (SEQ ID NO: 47) | | | | |
| 47 | TVFRSSPkRRSADIFI | 83 | 13.3 | 60 | *** |
| | (SEQ ID NO: 48) | | | | |
| 48 | TVFRSSEPRRSADIFI | 78 | 3351.633 | 5 | * |
| | (SEQ ID NO: 49) | | | | |
| 49 | TVFRSSEkPRSADIFI | 49 | — | 9 | |
| | (SEQ ID NO: 50) | | | | |
| 50 | TVFRSSEkRPSADIFI | 41 | — | 16 | |
| | (SEQ ID NO: 51) | | | | |
| 51 | TVFRSSEkRRPADIFI | 60 | — | 63 | |
| | (SEQ ID NO: 52 | | | | |
| 52 | TVFRSSEkRRSPDIFI | 45 | — | 75 | |
| | (SEQ ID NO: 53) | | | | |
| 53 | TVFRSSEkRRSAPIFI | 75 | — | 115 | |
| | (SEQ ID NO: 54) | | | | |
| 54 | TVFRSSEkRRSADPFI | 46 | — | 60 | |
| | (SEQ ID NO: 55) | | | | |
| 55 | TVFRSSEkRRSADIPI | 43 | — | 33 | |
| | (SEQ ID NO: 56) | | | | |
| 56 | TVFRSSEkRRSADIFP | 92 | 15.4 | 50 | *** |
| | (SEQ ID NO: 57) | | | | |
| 57 | T(Aib)FRSSEkRRSADIFI | 77 | 67.95 | 39 | *** |
| | (SEQ ID NO: 58) | | | | |
| 58 | TVF(Aib)SSEkRRSADIFI | 54 | 10000 | 11 | * |
| | (SEQ ID NO: 59) | | | | |
| 59 | TVFRS(Aib)EkRRSADIFI | 66 | — | 50 | |
| | (SEQ ID NO: 60) | | | | |
| 60 | TVFRSSE(Aib)RRSADIFI | 65 | 167.5 | 26 | **** |
| | (SEQ ID NO: 61) | | | | |
| 61 | TVFRSSEk(Aib)RSADIFI | 62 | 5214 | 25 | |
| | (SEQ ID NO: 62) | | | | |
| 62 | TVFRSSEkR(Aib)SADIFI | 51 | — | 25 | |
| | (SEQ ID NO: 63) | | | | |
| 63 | TVFRSSEkRR(Aib)ADIFI | 61 | — | 86 | |
| | (SEQ ID NO: 64) | | | | |
| 64 | TVFRSSEkRRS(Aib)DIFI | 60 | — | 105 | |
| | (SEQ ID NO: 65) | | | | |
| 65 | TVFRSSEkRRSA(Aib)IFI | 45 | — | 100 | |
| | (SEQ ID NO: 66) | | | | |
| 66 | TVFRSSEkRRSADI(Aib)I | 47 | 173 | 19 | **** |
| | (SEQ ID NO: 67) | | | | |
| 67 | TVFRSSEkRRSADIF(Aib) | 46 | — | 50 | |
| | (SEQ ID NO: 68) | | | | |
| 68 | GVFRSSEkRRSADIFI | 69 | 56.4 | 21 | ***** |
| | (SEQ ID NO: 69) | | | | |
| 69 | TGFRSSEkRRSADIFI | 77 | 38.1 | 50 | *** |
| | (SEQ ID NO: 70) | | | | |
| 70 | TVGRSSEkRRSADIFI | 81 | 52.5 | 29 | ***** |
| | (SEQ ID NO: 71) | | | | |
| 71 | TVFGSSEkRRSADIFI | 65 | 111.5 | 6 | **** |
| | (SEQ ID NO: 72) | | | | |
| 72 | TVFRGSEkRRSADIFI | 75 | 133.5 | 44 | ** |
| | (SEQ ID NO: 73) | | | | |
| 73 | TVFRSGEkRRSADIFI | 67 | — | 47 | |
| | (SEQ ID NO: 74) | | | | |
| 74 | TVFRSSGkRRSADIFI | 61 | — | 86 | |
| | (SEQ ID NO: 75) | | | | |
| 75 | TVFRSSEkGRSADIFI | 57 | — | 27 | |
| | (SEQ ID NO: 76) | | | | |
| 76 | TVFRSSEkRGSADIFI | 46 | — | 25 | |
| | (SEQ ID NO: 77) | | | | |
| 77 | TVFRSSEkRRGADIFI | 64 | — | 53 | |
| | (SEQ ID NO: 78) | | | | |
| 78 | TVFRSSEkRRSAGIFI | 60 | — | 62 | |
| | (SEQ ID NO: 79) | | | | |
| 79 | TVFRSSEkRRSADIGI | 39 | — | 43 | |
| | (SEQ ID NO: 80) | | | | |
| 80 | TVFRSSEkRRSADIFG | 35 | — | 52 | |
| | (SEQ ID NO: 81) | | | | |
| 81 | TVDRSSEyRRSADIFI | - | — | — | |
| | (SEQ ID NO: 82) | | | | |
| 82 | TVDRSSEKRRSADIFI | - | — | — | |
| | (SEQ ID NO: 83) | | | | |
| 87 | TVFQSEkRRSADQFI | - | 11 | 48 | *** |
| | (SEQ ID NO: 84) | | | | |

### AlphaScreen Cellular Kinase Assays.

GPCR activation results in modulation of downstream kinase systems and is often used to probe GPCR function and regulation. TGR Bioscience and PerkinElmer have developed Surefire cellular kinase assay kits that are HTS capable and useful in screening kinase regulation. Such kits enable the monitoring of Gi regulated downstream kinases like ERK1/2. The assay allows the measurement of increases in ERK1/2 kinase phosphorylation upon Gi coupled receptor (e.g., APJ) activation and this signal in turn can be used to assay Gi coupled receptor modulator. Similar kits are also available to assay other pathway dependent siganlling kinases such as MAP and BAD.

### β-Arrestin Signaling Assays.

Activation of the β-Arrestin signaling pathway was monitored using the commercially available DiscoveRx PathHunter® assay. This assay employs a homogenous, non-imaging assay format called Enzyme Fragment Complementation (EFC) using β-galactosidase (β-Gal) as the functional reporter. The enzyme is split into two inactive complementary portions (EA for Enzyme Acceptor and ED for Enzyme Donor) expressed as fusion proteins in the cell. EA is fused to β-Arrestin and ED is fused to the GPCR of interest. When the GPCR is activated and β-Arrestin is recruited to the receptor, ED and EA complementation occurs, restoring βGal activity which is measured using chemiluminescent PathHunter® Detection Reagent. Using this assay format, the endogenous ligand apelin-13 robustly recruits β-arrestin as measured by an increase in chemiluminescence.

Representative β-arrestin data are illustrated in Table 7. Conclusion: the compounds listed in Table 7 efficiently promote activation of Gi as exemplified by the potent inhibition of NKH477-stimulated cAMP production (EC50's<150 nM). In contrast, β-arrestin is weakly engaged for compounds 9, 82, and 87 and therefore the signaling is biased towards the Gi pathway. This bias has potential therapeutic advantage for the treatment of cardiovascular disease, including ischemia-reperfusion injury and heart failure. (ref: Scimia et al., Nature. 2012 Aug. 16; 488(7411):394-8)

TABLE 7

| | | Gi coupling | β-arrestin Signaling | |
|---|---|---|---|---|
| | | (cAMP inhibition)¹ | EC50 | Max |
| Cmpe # | Sequences | EC₅₀ (nM) | (uM) | Response² |
| Apelin-13 | N/A | 1 | 0.00165 | 101.7 |
| 9 | TVHRSSEkRRSADIFI | 79 | 2.2584 | 22 |
| | (SEQ ID NO: 10) | | | |
| 82 | TVDRSSEkRRSADIFI | 142 | 0.1685 | 20 |
| | (SEQ ID NO: 83) | | | |
| 87 | TVFQSEkRRSADQFI | 11 | 0.0344 | 47 |
| | (SEQ ID NO: 84) | | | |

¹Inhibition of NKH477 stimulated cAMP production in APJ-h HEK 293 cells
²Relative to apelin-13

In Vivo Assays

The in vivo efficacy of selected lead candidates is evaluated using animal models of cardiovascular function, heart failure, lipid metabolism, glucose homeostasis and body weight gain. Examples of such models are described below. However, it is noted that; the list of models described is not all encompassing, additional models are available and that these additional models may be used to exemplify the activity of our compounds.

### Cardiovascular Function

This model is commonly conducted in various wild type and genetically modified rodents, however, it is noted that such studies may also be conducted in in higher species such as dog, pig and non-human primate. The most commonly used rodent species are rat and mice. In addition to normal wild type rodents various genetic or environmental modifications have been used to create animals that better mimic various aspects of human disease. For example, the spontaneously hypertensive rat (commonly described as SHR rat) or rats fed on a high salt diet have elevated blood pressure akin to human hypertension.

It is noted that the use of large animals (typically dog or pig) allow for the fine placement of multiple measuring devices, which in turn allows for the measurement of multiple complex cardiovascular parameters. For example, placement of a balloon catheter into the left ventricle allows the measurement of both cardiac stroke volume and cardiac output. However, due to the small size of rats and mice such fine placement of measuring devices cannot be easily done. Consequently, in the rodent protocols described below we focus only on the measurement of crude cardiovascular parameters such as blood and heart rate.

It is also noted that measurement of cardiovascular function in rodents can be conducted in both anaesthetized and conscious animals. In conscious animals measurement devices are implanted surgically under anesthesia. Once the animal has regained consciousness these measurement devices send cardiovascular data from the animals to electronic recording stations. Such animals are commonly described as telemetered. Although this approach yields viable data its use can be hampered by data artifacts induced by handling of the animals, environmental factors and technical difficulties associated with the surgical implantation of the measuring devices. Consequently, the most common approach to measuring cardiovascular parameters in rodents is the use of anaesthetized animals. This approach is described in detail below.

Typically for anaesthetized models of cardiovascular function Wistar or CD rats, and C57Bl6 mice are used. However, it is noted that the procedure described below can be easily adapted for different strains of rat and mouse.

On the day of experimentation the animals are first anaesthetized. Common anesthetics used for rodents include Inactin or ketamine with xylazine. Once a level of full surgical anesthesia is confirmed the carotid or femoral artery is exposed and cannulated. This cannulae is typically filled with heparinized saline, to prevent blood clotting, and connected to a commercial pressure transducer such as those available from AD Instruments (adinstruments.com), to allow for the measurement of blood pressure. Typically, systolic and diastolic blood pressure will be recorded, and from this mean blood pressure and heart rate will be interpolated. To facilitate the intravenous administration of test substances the jugular vein may also be cannulated.

Using this model the effects of lead compounds on cardiovascular function can be evaluated. Typically the lead compound will be administered through; sub-cutaneous, oral, intra-peritoneal or intra-venous routes. To determine the effectiveness of our lead candidates the effects on blood pressure and heart rate in lead candidate treated animals will be compared to those observed in vehicle control treated animals.

### Heart Failure

A number of animal models of heart failure and myocardial injury have been reported in the literature. Such models include the use of large animals such as pig and dog, however, due to their small size and lower cost rodents are the most common species used. A useful review of rodent models of heart failure and myocardial injury can be found in the journal of Circulation—Heart Failure, 2009, Volume 2; pages 138 to 144. Such models require either; ligation of cardiac blood vessels or genetic manipulation (such as cardiomyocyte specific overexpression of TNFα).

The spontaneous hypertensive rat (SHR) is an especially useful acute rat models of chronic ventricular pressure overload whereas the mean arterial pressure in male wild type Wistar or SHR rats can be accessed via cannulation of the femoral or carotid (Regul. Pept. 2001:99:87). In addition, isolated rat heart preps have been used to demonstrate the inotropic effects of apelin and therefore could be used to evaluate APJ agonists, antagonists, or modulators (Circ. Res 2002; 91(5):434-40) For a more chronic model of heart failure, a useful exemplary model is the aortic banding methodology in the mouse (Circ Res. 2007:101:e32-e42).

Mouse and rat Langendorff heart preparations were used to characterize the direct cardiac effects apelin and APJ compounds have on the target tissue (Szokodi, Circ. Res 2002:91 434-440). Hearts were perfused with (in mM) 118 NaCl, 4.7 KCl, 1.2 KH2PO4, 1.5 CaCl₂, 1.2 MgCl₂, 23 NaHCO₃, and 10.0 dextrose, gassed with 95% O₂-5% CO₂ and adjusted to a pH of 7.4. A pressure-sensing balloon catheter was inserted in the LV cavity to record changes in the developed pressure. Heart function was assessed by measuring standard parameters such as heart rate, mean peak systolic pressure, mean end diastolic pressure, developed pressure, dP/dt max, and dP/dt min An increase in developed pressure is consistent with the known inotropic effect of the endogenous ligand for APJ, apelin.

### Lipolysis and Body Weight

This model is most commonly conducted in a variety of mouse strains, however, it is noted that such studies may also be conducted in rats and in higher species such as dog and non-human primate. For mice, the most commonly used strain is the C57Bl6. For these C57Bl6 mice, the ability of compounds to modify lipid metabolism (lipolysis) may be evaluated in animals fed a standard laboratory diet, or they may be evaluated in mice fed a modified high calorie diet. Mice fed a high calorie diet are commonly described as DIO (diet induced obesity) because such a diet induces a disease state that shares a number of characteristics with human cardiovascular and metabolic disease. Such DIO mice have elevated blood lipid levels, similar to makers of human heart disease, cardiovascular disease, and atherosclerosis.

When mice fed on standard laboratory diet are used, the animals are typically 8 to 16 weeks of age when used for experimentation. When mice fed on a high calorie diet are used, the animals are typically 13 to 16 weeks of age (equating to 7 weeks feeding with standard laboratory diet, followed by 6 to 9 weeks feeding with a high calorie diet).

The activity of test compounds will be evaluated in mice where lipid metabolism is first elevated by either overnight fasting or by pretreatment with a pharmacological stimulator of lipid metabolism (typically isoproterenol). Most typically overnight fasting will be used to elevate lipid metabolism prior to testing our lead candidates, consequently, this is the protocol described in detail below.

One day prior to the day of experimentation the mice are fasted overnight (minimum of 12 hours and maximum of 18 hours) to elevate lipid metabolism. During this period the mice are allowed free access to drinking water, but the food is removed.

On the day of experimentation the test compound will be administered through; sub-cutaneous, oral, intra-peritoneal or intra-venous routes. After a predefined period (typically 5 to 360 minutes dependent on the pharmacokinetic properties of the lead candidate) a blood sample will be obtained, typically through terminal cardiac puncture. Blood will be transferred into a tube with a suitable anti-coagulant (typically heparin or EDTA), and centrifuged to separate blood cells from plasma. Plasma will then be harvested. The concentration of blood lipids (typically glycerol and non-esterified free fatty acid) will then be determined in these plasma samples using commercial assay kits such as those available from Zen Bio (www.zen-bio.com).

To determine the effectiveness of test compound the profile of blood lipids in mice receiving test compound will be compared to that observed in vehicle control mice.

The protocol described above assesses the activity of test compounds after a single acute dose. This protocol can be adapted to determine the activity of test compounds after chronic administration. In this situation the compound will be administered once or multiple times during a 24 hour period, and for periods extending up to 3 months. During the course of compound dosing, or at the end of a predefined period of compound dosing, the animal's response to fasting-induced lipolysis will be determined using the protocol laid out in the above section. In addition, throughout the course of chronic compound dosing the body weight of the animals will also be recorded. Comparison of the change in body weight over time for compound treated vs. vehicle treated animals will elucidate any compound-mediated effect on body weight.

### Glucose Tolerance and Body Weight

This model is most commonly conducted in a variety of mouse strains, however, it is noted that such studies may also be conducted in rats and in higher species such as dog and non-human primate. For mice, the most commonly used strain is the C57Bl6. For these C57Bl6 mice, the ability of test compounds to modify glucose homeostasis may be evaluated in animals fed a standard laboratory diet, or they may be evaluated in mice fed a modified high calorie diet. Mice fed a high calorie diet are commonly described as DIO (diet induced obesity) because such a diet induces a disease state that shares a number of characteristics with human metabolic and cardiovascular disease. Such DIO mice have a compromised ability to regulate blood glucose, similar to human type II diabetes.

When mice fed on standard laboratory diet are used, the animals are typically 8 to 16 weeks of age when used for experimentation. When mice fed on a high calorie diet are used, the animals are typically 13 to 16 weeks of age (equating to 7 weeks feeding with standard laboratory diet, followed by 6 to 9 weeks feeding with a high calorie diet).

One day prior to the day of experimentation the mice are fasted overnight (minimum of 12 hours and maximum of 18 hours). During this period the mice are allowed free access to drinking water, but the food is removed.

On the day of experimentation a small drop of blood (5 to 10 μl) will be obtained from the animal's tail and applied to a calibrated commercial glucometer to determine basal blood glucose concentration. Subsequently, the test compound will be administered through; sub-cutaneous, oral, intra-peritoneal or intra-venous routes. After a predefined period (typically 5 to 360 minutes dependent on the pharmacokinetic properties of the lead candidate) a further blood sample will be obtained from the tail and blood glucose concentration determined Immediately afterwards the mice will be given a single glucose challenge. Typically, this glucose challenge will be in the form of a solution of D-glucose administered via oral, intra-peritoneal or intra-venous routes. Subsequent to this glucose challenge, blood glucose will be measured, at various time intervals using the same tail bleed/glucometer method. Typically, blood glucose will be measured at 30, 60 and 120 minutes after the glucose challenge.

To determine the effectiveness of test compounds, the profile of blood glucose concentration over time observed in mice receiving test compound will be compared to that observed in vehicle control mice.

The protocol described above assesses the activity of test compounds after a single acute dose. We may also adapt this protocol to determine the activity of test compounds after chronic administration. In this situation the compound will be administered once or multiple times during a 24 hour period, and for periods extending up to 3 months. During the course of compound dosing, or at the end of a predefined period of compound dosing, the animal's response to a glucose challenge will be determined using the protocol laid out in the above section. In addition, throughout the course of chronic compound dosing the body weight of the animals will also be recorded. Comparison of the change in body weight over time for compound treated vs. vehicle treated animals will elucidate any compound-mediated effect on body weight.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. The compound represented by Formula A:
T-L-X₁-X₂-X₃-X₄-X₅-X₆-X₇-X₈-X₉-X₁₀-X₁₁-X₁₂-X₁₃-X₁₄-X₁₅-X₁₆-R₁;
or a pharmaceutically acceptable salt thereof, wherein L is a linking moiety bonded to the N terminal nitrogen of X₁ or the next present amino acid residue if X₁ is absent and is selected from: C(O), C(S), S(O)₂, N(R¹³)S*(O), N(R¹³)S*(O)₂, N(R¹³)C*(O), N(R¹³)C*(S), OC*(O), OC*(S), SC*(O), SC*(S), C(═NH), and N(R¹³)C*(═NH); wherein L is bonded to X₁ or the next present amino acid residue if X₁ is absent at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; T is a lipophilic moiety bonded to L; R₁ is —OR₂—, or NR₃R₄; wherein each R₂ is hydrogen or a (C₁-C₁₀) alkyl group,
R₃ and R₄ are each independently selected from hydrogen, (C₁-C₁₀) alkyl, (C₁-C₁₀)aralkyl, [CH₂CH₂O]ₙCH₂CH₂C(O)OR₂ or [CH₂CH₂O]ₙCH₂CH₂C(O)NR₂;
n is 1-20
or —NR₃R₄ is a non-aromatic nitrogen-containing heterocyclic group, wherein the non-aromatic group is optionally mono-or di-substituted at one or more substitutable carbon atoms with an R₅;
each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂;
X₁ is absent, a threonine residue, a histidine residue, a glutamine residue, an aspartic acid residue, an Aib residue, a phenylalanine residue, or a glycine residue;
X₂ is absent, a valine residue, a phenylalanine residue, a histadine residue, an Aib residue, a glutamine residue, an aspartic acid residue, a proline residue, or a glycine residue;
X₃ is absent, a phenylalanine residue, a histidine residue, an aspartic acid residue, a glycine residue, or a proline residue;
X₄ is absent, an arginine residue, a phenylalanine residue, a histidine residue, a glutamine residue, an aspartic acid residue, an Aib residue, a proline residue, or a glycine residue;
X₅ is absent, a serine residue, a phenylalanine residue, a histidine residue, a glutamine residue, an aspartic acid residue, a proline residue, an Aib residue, or a glycine residue;
X₆ is absent, a serine residue, a phenylalanine residue, a histidine residue, a glutamine residue, an aspartic acid residue, a proline residue, an Aib residue, or a glycine residue;
X₇ is absent, a glutamic acid, a phenylalanine residue, a histidine residue, a glutamine residue, an aspartic acid residue, a proline residue, an Aib residue, or a glycine residue;
X₈ is absent, a lysine, D-lysine residue, a phenylalanine residue, a proline residue, a glutamic acid, a phenylalanine residue, a histidine residue, a glutamine residue, an aspartic acid residue, an Aib residue, or a glycine residue, or a tyrosine residue;
X₉ is absent, an arginine residue, a phenylalanine residue, a histidine residue, a glutamine residue, an aspartic acid residue, a proline residue, an Aib residue, or a glycine residue;
X₁₀ is absent, an arginine residue, a phenylalanine residue, a histidine residue, a glutamine residue, an aspartic acid residue, a proline residue, an Aib residue, or a glycine residue;
X₁₁ is absent, a serine residue, a phenylalanine residue, a histidine residue, a glutamine residue, an aspartic acid residue, a proline residue, an Aib residue, or a glycine residue;
X₁₂ is absent, an alanine residue, a phenylalanine residue, a histidine residue, a glutamine residue, an aspartic acid residue, a proline residue, an Aib residue, or a glycine residue;
X₁₃ is absent, an aspartic acid residue, a phenylalanine residue, a histidine residue, a glutamine residue, an aspartic acid residue, a proline residue, an Aib residue, or a glycine residue;
X₁₄ is absent or an isoleucine residue, a phenylalanine residue, a histidine residue, a glutamine residue, an aspartic acid residue, a proline residue, an Aib residue, or a glycine residue;
X₁₅ is absent, a phenylalanine residue, a histidine residue, a glutamine residue, an aspartic acid residue, a proline residue, an Aib residue, or a glycine residue;
X₁₆ is absent, a phenylalanine residue, an isoleucine residue, a phenylalanine residue, a histidine residue, a glutamine residue, an aspartic acid residue, a proline residue, an Aib residue, or a glycine residue;
wherein at least three of X₁-X₁₆ are present and contiguous and optionally 1-5 amino acid residues are present in the D configuration.

2. The compound represented by Formula B:
T-L-X₁′-X₂-X₃-X₄-X₅-X₆-X₇-X₈-X₉-X₁₀-X₁₁-X₁₂-X₁₃-X₁₄-X₁₅-X₁₆-R₁;
or a pharmaceutically acceptable salt thereof, wherein L is a linking moiety bonded to the N terminal nitrogen of X₁′ or the next present amino acid residue if X₁′ is absent and is selected from: C*(O), C*(S), S*(O)₂, N(R¹³)S*(O), N(R¹³)S*(O)₂, N(R¹³)C*(O), N(R¹³)C*(S), OC*(O), OC*(S), SC*(O), SC*(S), C*(═NH), and N(R¹³)C*(═NH); wherein L is bonded to X₁′ or the next present amino acid residue if X₁′ is absent at the atom marked with an asterisk (*) and R¹³ is selected from: H, D, (C₁-C₆)alkyl, (C₂-C₆)alkenyl, (C₂-C₆)alkynyl, (C₁-C₆)alkoxy, (C₃-C₉)cycloalkyl, 5-10 membered heterocycloalkyl, aryl, aryloxy, heteroaryloxy, aralkyl, heteroaryl, and heteroaralkyl; wherein said alkyl, alkenyl, alkynyl, alkoxy, cycloalkyl, heterocycloalkyl, aryl, aryloxy, heteroaryloxy, aralkyl, heteroaryl, and heteroaralkyl are optionally and independently substituted; T is a lipophilic moiety bonded to L; R₁ is —OR₂—, or NR₃R₄; wherein each R₂ is hydrogen or a (C₁-C₁₀) alkyl group,
R₃ and R₄ are each independently selected from hydrogen, (C₁-C₁₀) alkyl, (C₁-C₁₀)aralkyl, [CH₂CH₂O]ₙCH₂CH₂C(O)OR₂ or [CH₂CH₂O]ₙCH₂CH₂C(O)NR₂;
n is 1-20
or —NR₃R₄ is a non-aromatic nitrogen-containing heterocyclic group, wherein the non-aromatic group is optionally mono-or di-substituted at one or more substitutable carbon atoms with an R₅;
each R₅ is independently halogen, —OH, C₁-C₃ alkyl, C₁-C₃ haloalkyl, —NO₂, —C₁-C₃ alkoxy, —C₁-C₃ haloalkoxy, —CN, —NH₂, —C₁-C₃ alkylamino, —C₁-C₃ dialkylamino, —C(O)NH₂, —C(O)NH(C₁-C₃ alkyl), —C(O)(C₁-C₃ alkyl), —NHC(O)(C₁-C₃ alkyl), —NHC(O)H, —C(O)N(C₁-C₃ alkyl)₂, —NHC(O)O—(C₁-C₃ alkyl), —C(O)OH, —C(O)O—(C₁-C₃ alkyl), —NHC(O)NH₂, —NHC(O)NH(C₁-C₃ alkyl), —NHC(O)N(C₁-C₃ alkyl)₂, or —SO₂NR₂;
X₁′ is absent, a threonine residue, a histidine residue, a glutamine residue, an aspartic acid residue, an Aib residue, a phenylalanine residue, a glycine residue, or a proline residue;
X₂ is absent, a valine residue, a phenylalanine residue, a histadine residue, an Aib residue, a glutamine residue, an aspartic acid residue, a proline residue, or a glycine residue;
X₃ is absent, a phenylalanine residue, a histidine residue, an aspartic acid residue, a glycine residue, or a proline residue;
X₄ is absent, an arginine residue, a phenylalanine residue, a histidine residue, a glutamine residue, an aspartic acid residue, an Aib residue, a proline residue, or a glycine residue;
X₅ is absent, a serine residue, a phenylalanine residue, a histidine residue, a glutamine residue, an aspartic acid residue, a proline residue, an Aib residue, or a glycine residue;
X₆ is absent, a serine residue, a phenylalanine residue, a histidine residue, a glutamine residue, an aspartic acid residue, a proline residue, an Aib residue, or a glycine residue;
X₇ is absent, a glutamic acid, a phenylalanine residue, a histidine residue, a glutamine residue, an aspartic acid residue, a proline residue, an Aib residue, or a glycine residue;
X₈ is absent, a lysine, D-lysine residue, a phenylalanine residue, a proline residue, a glutamic acid, a phenylalanine residue, a histidine residue, a glutamine residue, an aspartic acid residue, an Aib residue, or a glycine residue, or a tyrosine residue;
X₉ is absent, an arginine residue, a phenylalanine residue, a histidine residue, a glutamine residue, an aspartic acid residue, a proline residue, an Aib residue, or a glycine residue;
X₁₀ is absent, an arginine residue, a phenylalanine residue, a histidine residue, a glutamine residue, an aspartic acid residue, a proline residue, an Aib residue, or a glycine residue;
X₁₁ is absent, a serine residue, a phenylalanine residue, a histidine residue, a glutamine residue, an aspartic acid residue, a proline residue, an Aib residue, or a glycine residue;
X₁₂ is absent or an alanine residue, a phenylalanine residue, a histidine residue, a glutamine residue, an aspartic acid residue, a proline residue, an Aib residue, or a glycine residue;
X₁₃ is absent, an aspartic acid residue, a phenylalanine residue, a histidine residue, a glutamine residue, an aspartic acid residue, a proline residue, an Aib residue, or a glycine residue;
X₁₄ is absent, an isoleucine residue, a phenylalanine residue, a histidine residue, a glutamine residue, an aspartic acid residue, a proline residue, an Aib residue, or a glycine residue;
X₁₅ is absent, a phenylalanine residue, a histidine residue, a glutamine residue, an aspartic acid residue, a proline residue, an Aib residue, or a glycine residue;
X₁₆ is absent, a phenylalanine residue, an isoleucine residue, a phenylalanine residue, a histidine residue, a glutamine residue, an aspartic acid residue, a proline residue, an Aib residue, or a glycine residue;
wherein at least three of X₁′-X₁₆ are present and contiguous and optionally 1-5 amino acid residues are present in the D configuration.

3. The compound of claim 1, wherein
X₁ is absent, a threonine residue, a glutamine residue, an aspartic acid residue, a glycine residue;
X₂ is absent, a valine residue, an Aib residue, a glutamine residue, or a glycine residue;
X₃ is absent, a phenylalanine residue, a glycine residue, an aspartic acid residue or a histidine residue;
X₄ is absent, an arginine residue, an Aib residue, a proline residue, or a glycine residue;
X₅ is absent, a serine residue or a glycine residue;
X₆ is absent, a serine residue, or a histidine residue;
X₇ is absent, a glutamic acid residue, or a proline residue;
X₈ is D-lysine residue or a proline residue;
X₉ is absent, an arginine residue, phenylalanine residue, an Aib residue, or a glutamine residue;
X₁₀ is absent, an arginine residue or a histidine residue;
X₁₁ is absent, a serine residue or an aspartic acid residue;
X₁₂ is absent or an alanine residue;
X₁₃ is absent or an aspartic acid residue;
X₁₄ is absent or an isoleucine residue, a glutamine residue or an aspartic acid residue;
X₁₅ is absent or a phenylalanine residue, or an Aib residue; and
X₁₆ is absent or an isoleucine residue, a phenylalanine residue, or a proline residue.

4. The compound of any one of claims 1-2, wherein at least nine of X₁-X₁₆ or X₁′-X₁₆ are present.

5. The compound of any one of claims 1-2, wherein X₁-X₁₆ or X₁′-X₁₆ are present.

6. The compound of claim 4, wherein X₈ is D-lysine.

7. The compound of 6 claim 4, wherein X₁ is a glutamine residue, an aspartic acid residue, or a glycine residue.

8. The compound of claim 4, wherein X₃ is a glycine residue or an aspartic acid residue.

9. The compound of claim 4, wherein X₄ is a proline residue or a glycine residue.

10. The compound of claim 4, wherein X₆ is a glutamine residue.

11. The compound of claim 4, wherein X₉ is a phenylalanine residue, a glutamine residue, or an Aib residue.

12. The compound of claim 4, wherein X₁₀ is a histidine residue.

13. The compound of claim 4, wherein X₁₅ is an Aib residue.

14-16. (canceled)

17. A compound of claim 1 selected from any one of Compound Nos. 1-82 and 87 or a pharmaceutically acceptable salt thereof.

18. The compound of claim 17 selected from any one of Compound Nos. 1, 20, 24, 68 and 70 or a pharmaceutically acceptable salt thereof.

19. (canceled)

20. (canceled)

21. A compound of claim 1 selected from:
Compound No. 1: 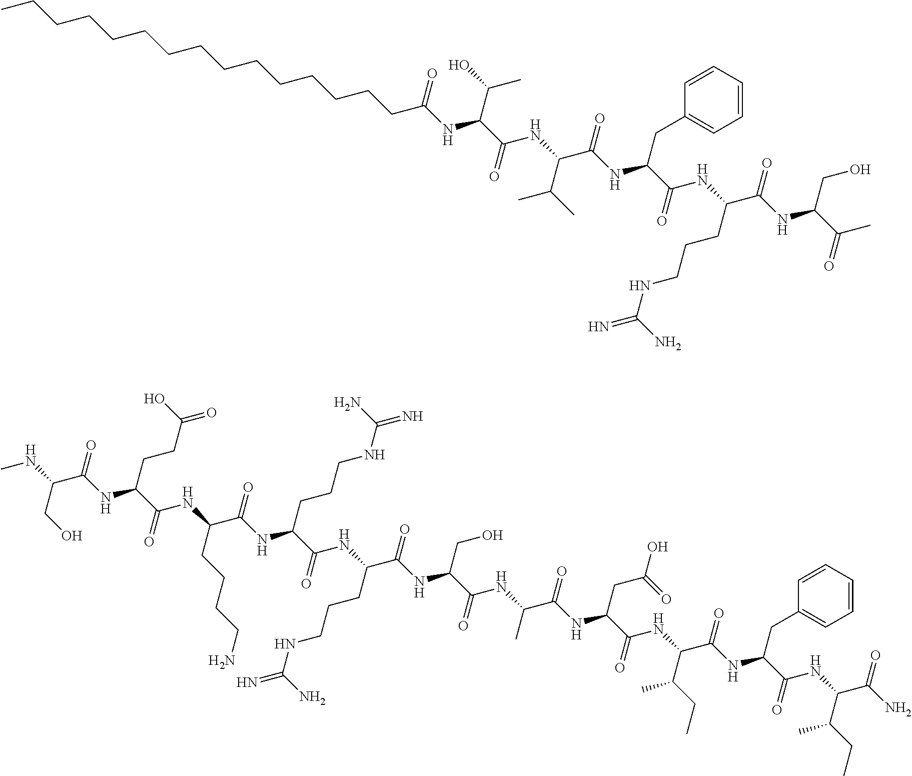
or a pharmaceutically acceptable salt thereof.

22-24. (canceled)

25. The compound of claim 4, wherein T is an optionally substituted (C₆-C₃₀)alkyl, (C₆-C₃₀)alkenyl, (C₆-C₃₀)alkynyl, wherein 0-3 carbon atoms are replaced with oxygen, sulfur, nitrogen or a combination thereof.

26. The compound of claim 25, wherein T is selected from the group consisting of: CH₃(CH₂)₁₆, CH₃(CH₂)₁₅, CH₃(CH₂)₁₄, CH₃(CH₂)₁₃, CH₃(CH₂)₁₂, CH₃(CH₂)₁₁, CH₃(CH₂)₁₀, CH₃(CH₂)₉, CH₃(CH₂)₈, CH₃(CH₂)₉OPh-, CH₃(CH₂)₆C═C(CH₂)₆, CH₃ (CH₂)₁₁O(CH₂)₃, and CH₃(CH₂)₉O(CH₂)₂.

27. The compound of claim 4, wherein T is selected from: a fatty acid derivative; a bile acid derivative; sterols; progestagens; glucocorticoids; mineralcorticoids; androgens; and estrogens.

28-31. (canceled)

32. The compound of claim 4, wherein T is selected from:

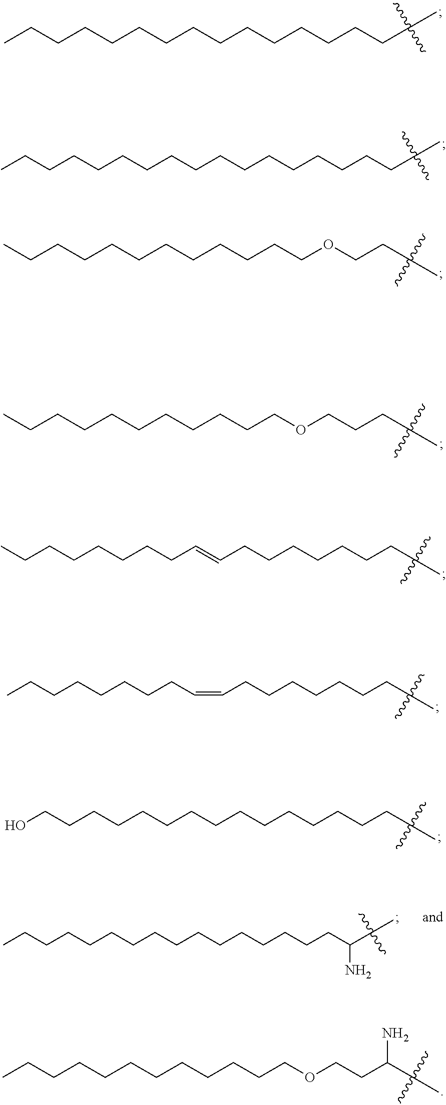

33. The compound of claim 4, wherein TL is selected from:
CH₃(CH₂)₁₅—C(O);
CH₃(CH₂)₁₃—C(O);
CH₃(CH₂)₉O(CH₂)₂C(O);
CH₃(CH₂)₁₀O(CH₂)₂C(O);
CH₃(CH₂)₆C═C(CH₂)₆—C(O);
LCA-C(O); and
CH₃(CH₂)₉OPh-C(O) wherein

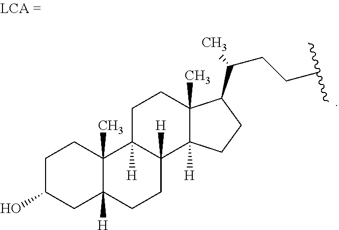

34. A method of treating cardiovascular disease, cancer, diabetes, stem cell trafficking, fluid homeostasis, cell proliferation, immune function, obesity, metastatic disease, and HIV infection in a patient in need thereof comprising administering to said patient and effective amount of a compound of claim 4.

35-36. (canceled)

37. A pharmaceutical composition, comprising a compound of claim 4 and a pharmaceutically acceptable carrier.
